(19) United States
(12) Patent Application Publication
(10) Pub. No.: US 2026/0242390 A1
(43) Pub. Date: Aug. 20, 2026

(54) TOTAL SYNTHESES OF PORTIMINE A AND ANALOGUES THEREOF AS SMALL MOLECULE MODULATORS OF NMD3

(71) Applicant: THE SCRIPPS RESEARCH INSTITUTE, La Jolla, CA (US)

(72) Inventors: Phil S. BARAN, San Diego, CA (US); Christopher PARKER, Palm Beach Gardens, FL (US); Luke L. LAIRSON, San Diego, CA (US); Junchen TANG, San Diego, CA (US); Weichao LI, San Diego, CA (US); Francisco MARTINEZ, San Diego, CA (US); Tzu-Yuan CHIU, San Diego, CA (US)

(21) Appl. No.: 19/147,893

(22) PCT Filed: Jan. 17, 2024
(86) PCT No.: PCT/US2024/011792

§ 371 Date: Jul. 14, 2025

Related U.S. Application Data
(60) Provisional application No. 63/480,259, filed on Jan. 17, 2023.

(51) Int. Cl. C07D 491/22

(52) U.S. Cl. CPC C07D 491/22

(57) ABSTRACT

Disclosed herein are enantioselective methods of making known cyclic imine (CI) toxins and analogues thereof as small molecule inhibitors of NMD3 with selective and potent anti-cancer activity.

### CROSS-REFERENCE

This application claims the benefit of U.S. Provisional Application No. 63/480,259, filed on Jan. 17, 2023, which is incorporated herein by reference in its entirety.

### GOVERNMENT SUPPORT

This invention was made with government support under GM118176 awarded by the National Institutes of Health. The government has certain rights in the invention.

### FIELD OF THE INVENTION

Disclosed herein are enantioselective synthetic methods of making known cyclic imine (CI) toxins and analogues thereof as small molecule inhibitors of NMD3 with selective and potent anti-cancer activity.

### BACKGROUND OF THE INVENTION

For decades, cyclic imine (CI) toxins have stimulated extensive interest from the broad scientific community based on their unique and potent bioactivity coupled with their captivating chemical structures.^{1,2} The therapeutic potential of larger members in this family, such as pinnatoxins,³ spirolides,⁴ and gymnodimines,⁵ have been thwarted by their high neurotoxicity in vivo (LD₅₀<100 μg/kg). More compact members of this family were isolated from benthic dinoflagellate Vulcanodinium rugosum in 2013 and 2018, portimine A (1) and B (2) respectively (FIG. 1, absolute configuration confirmed in 2019).⁶⁻⁸ In sharp contrast to classic CI toxins, 1 exhibits a promising therapeutic index exibiting lower toxicity (LD₅₀=1570 μg/kg) coupled with potent bioactivity.^{6,9-12} Indeed, 1 demonstrates potent anticancer,⁶ antifouling,¹⁰ and anti-HIV-1 activity,¹¹ making it a promising lead compound. The Achilles heel of such a lead compound is of course scalable access to its complex architecture. As it is derived from a dinoflagellate in low yield,⁶ chemical synthesis appears to be the only means of realistically procuring such molecules.

Even if this could be achieved, semi-synthetic analogs with deep-seated modifications would be inaccessible. Featuring a spiro-fused five-membered cyclic imine embedded in a highly oxidized all-carbon tricyclic macrocyclic core, 1 and 2 are formidable targets for synthesis. The unusual peripheral oxidations such as that adjacent to imine carbon (C-5) and neighboring labile medium-sized cyclic ketal add to this challenge.

Historically, CI toxins have been constructed by patterning the retrosynthetic analysis on the presumed biogenesis¹³⁻¹⁵ wherein an acyclic structure with maximum functionality is subjected to macrocyclization.¹⁶⁻²⁰ This pioneering approach was first accomplished by Kishi et al. in the 1998 total synthesis of pinnatoxin A.¹⁶ In the case of the portimines, approaches thus far have followed this dogma.²¹⁻²⁴ Thus, Brimble et al.,^{21, 22} and Harran et al.²³ aimed for a bio-inspired synthesis that mimics the polyketide synthases (PKSs), featuring bold intramolecular cyclizations of fully-functionalized polyketides 3 and 4 respectively. The former approach demonstrated that the ketalization of linear 3 was not facile, even with the well-functionalized skeleton. The latter Diels-Alder based approach resulted in undesired regioselectivity in the pivotal cyclization of 4 to 6.

The difficulty encountered in these routes points to the challenge of forging key bonds in such a densely functionalized polycyclic alkaloid from an acyclic precursor. From a high level, this scenario is not unlike that encountered in the synthesis of densely functionalized, highly oxidized terpene natural products. In those cases, it has been shown that a two-phase approach to synthesis can be beneficial by building up a minimally oxidized carbon framework followed by strategic late-stage oxidations.²⁵⁻³⁰

Thus, there exists a need in the field for improved methods of synthesizing cyclic imine toxins toxins and analogues thereof as small molecule inhibitors of NMD3 with selective and potent anti-cancer activity.

### BRIEF DESCRIPTION OF THE FIGURES

FIG. 1. Portimine A is non-toxic to primary human PBMCs.

FIG. 2. Washout experiment reveals PA has acute cytotoxicity.

FIG. 3. Initial SAR studies: Assessing activity of PAL functionalized PTMA analogs.

FIG. 4. Validation of NMD3 as target of Portimine A by Chemo-precipitation (ChP).

FIG. 5. PA related compounds induce cell apoptosis in Jurkats.

FIG. 6. Portimine A inhibits S phase entry in Jurkat cells.

FIG. 7. NMD3 is downregulated by PA in Jurkat cells in a time- and dose-dependent manner.

FIG. 8. NMD3 down regulation is not occurring through proteosome-mediated degradation.

FIG. 9. Portimine A displays potent anti-tumor activity in human and syngeneic mouse tumor models.

### SUMMARY OF THE INVENTION

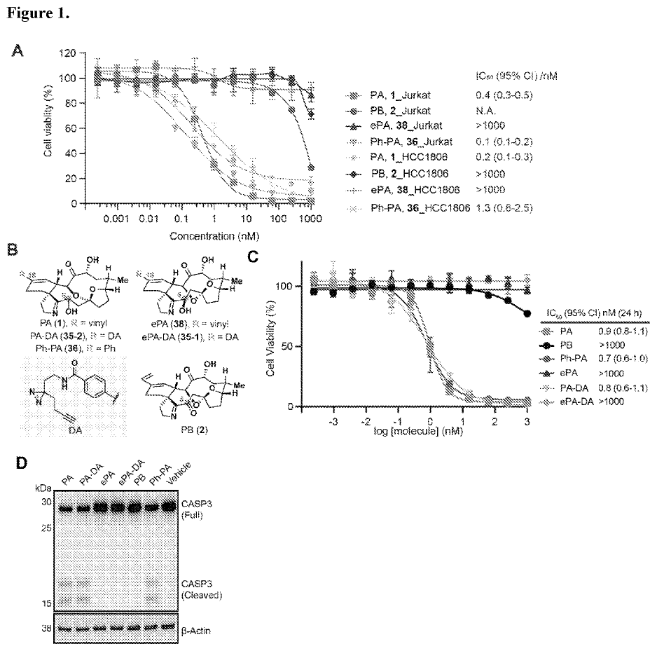

Disclosed herein are enantioselective synthetic methods of making Portimine A and analogues thereof as small molecule inhibitors of NMD3 with selective and potent anti-cancer activity. Notably, Portimine A was found to be non-toxic to primary human PBMCs (FIG. 1) despite high potency in multiple cancer cell lines.

The application provides a process for preparing Portimine A (1),

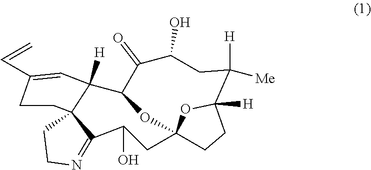

the process comprising the steps of:
i) the reduction of Compound (2) to form Portimine A (1):

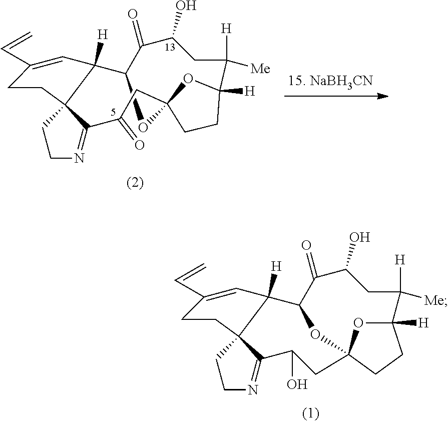

ii) the Dess-Martin oxidation of Compound (22) and subsequent hydrolysis to form Compound (2)

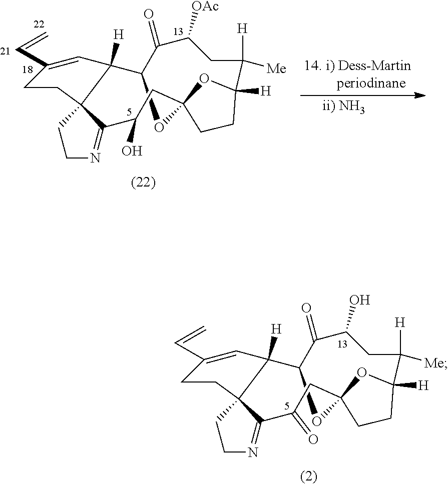

iii) the Pd-catalyzed vinylation of Compound (21) to form Compound (22):

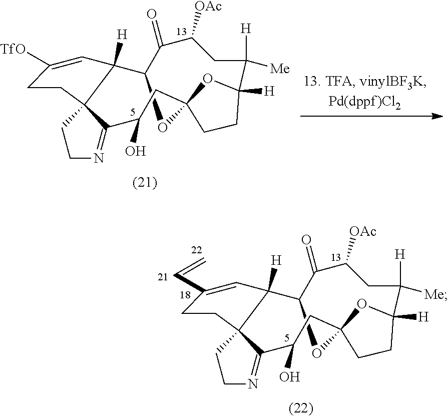

iv) the conversion of Compound (20) to Compound (21):

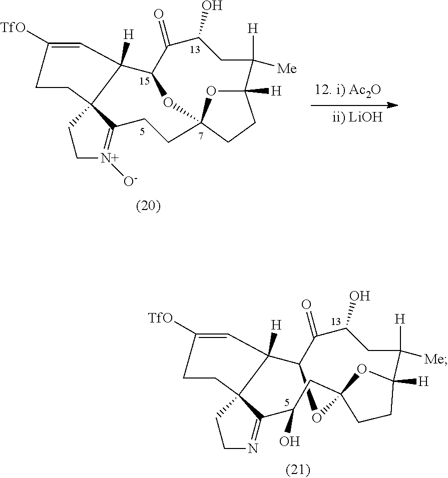

v) the silylation and oxidation of Compound (19) to form Compound (20):

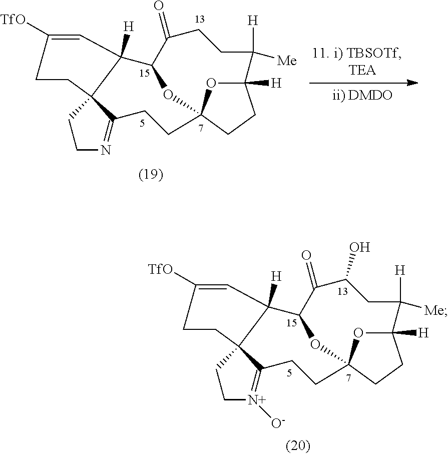

vi) ring-chain tautomerism of Compound (32) to form Compound (19):

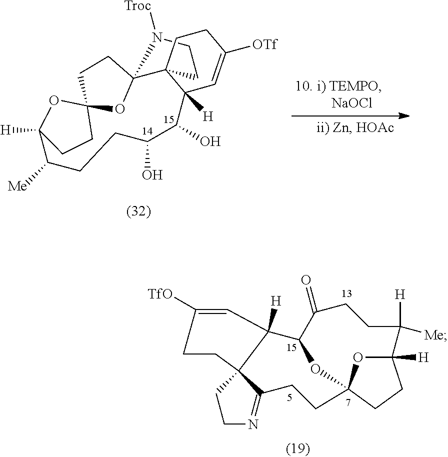

vii) the reduction of Compound (16) to Compound (32):

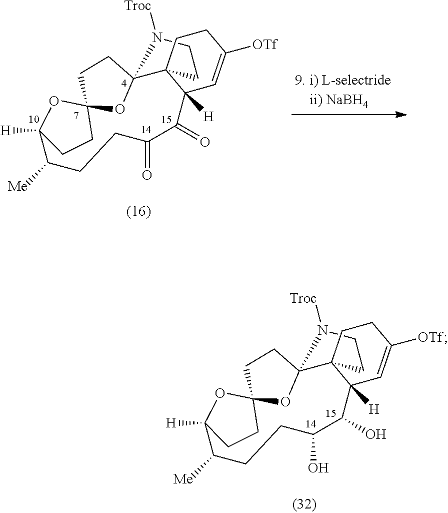

viii) ring formation and oxidation of Compound (15) to form Compound (16):

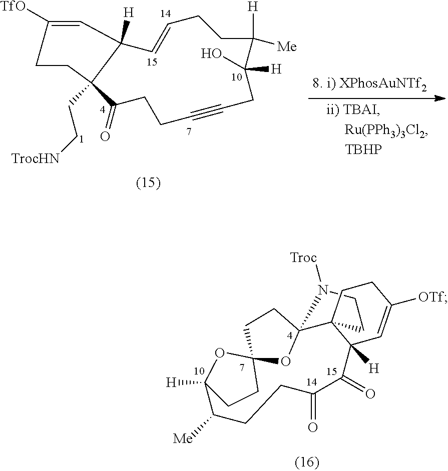

ix) Mo-catalyzed ring-closing alkyne metathesis to form Compound (15):

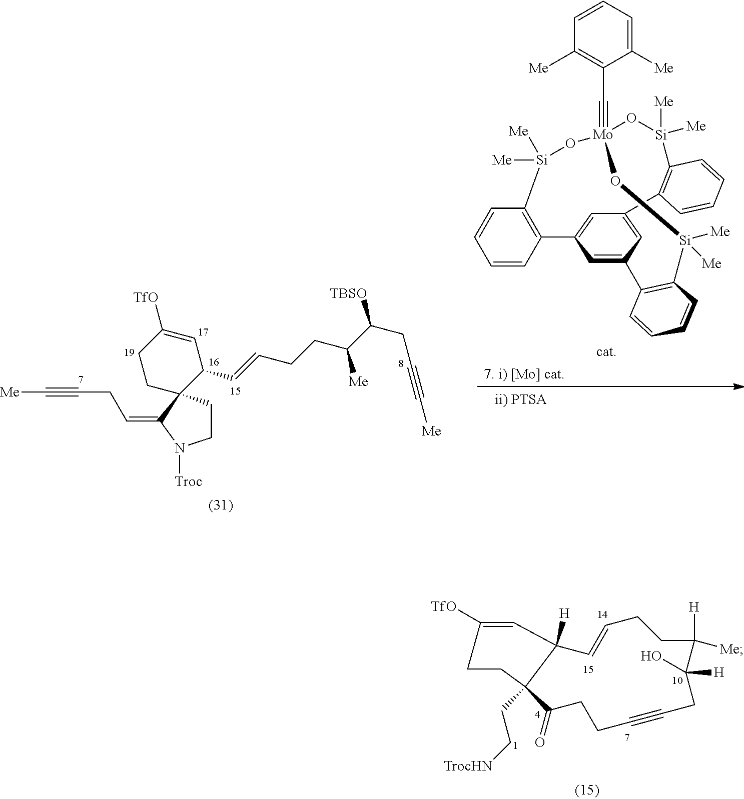

x) the conversion of compound (7) to the N-protected imine Compound (31):

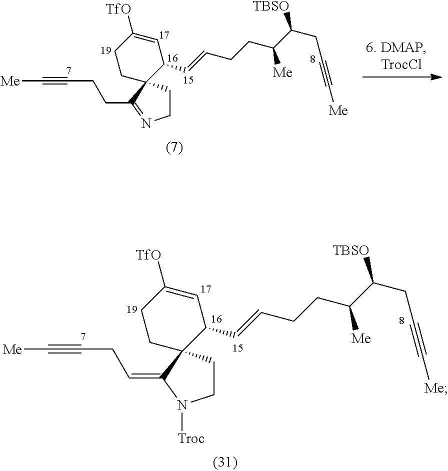

xi) the transmetallation of Compound (13) and conjugate addition with Compound (12) to form Compound (7):

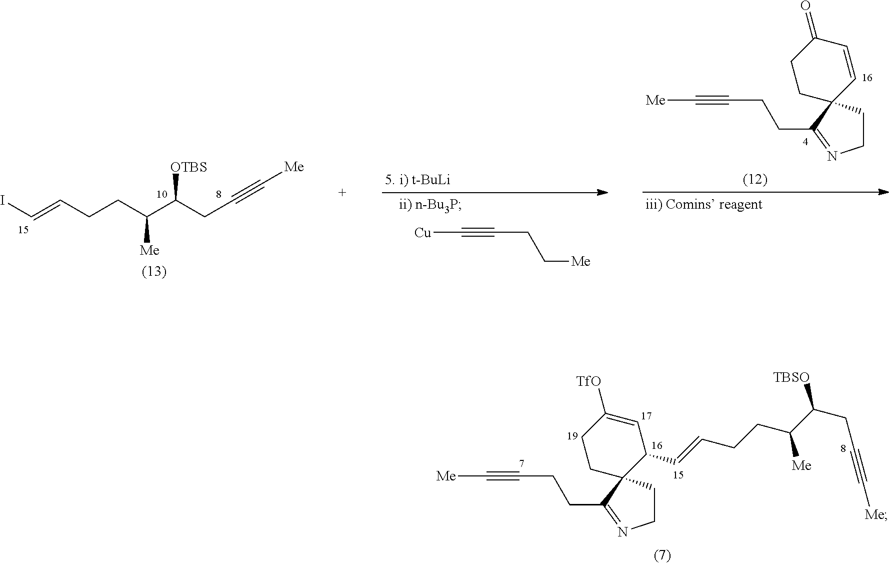

xii) the cyclization of Compound (11) to form Compound (12):

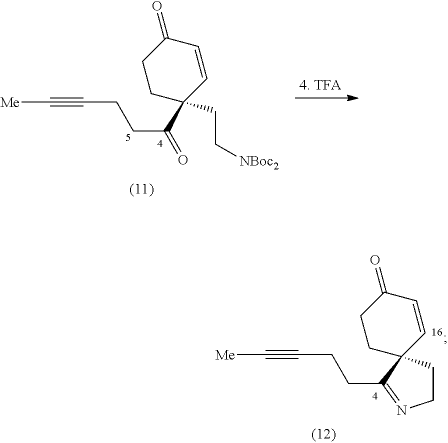

xiii) the alcohol oxidation of Compound (10), subsequent Grignard addition, and Dess-Martin oxidation to form Compound (11):

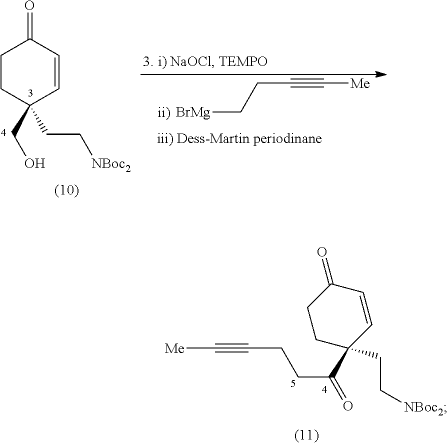

xiv) Diels-Alder cycloaddition of Compound (8) to form Compound (10):

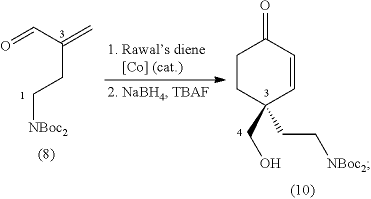

and
xv) conversion of Compound (23) to form Compound (8):

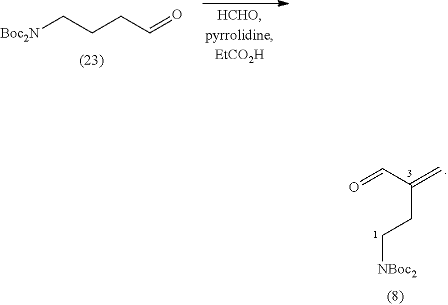

The application provides a process of preparing diverse analogues of Portimine A, comprising the following steps:

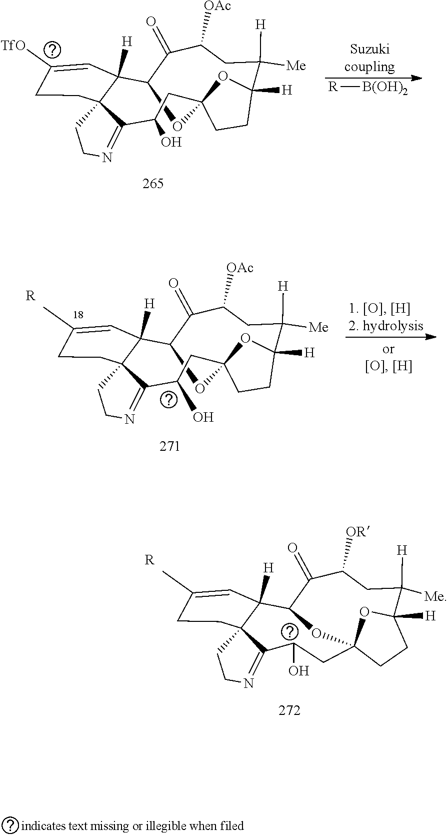

wherein:
R is (C₆-C₁₀) aryl, (C₂-C₆) alkenyl, or (C₅—Cl₂) heteroaryl, wherein each aryl is optionally substituted with —NH(CH₂)₂C(—N═N—)(CH₂)₂(C≡CH); and
R′ is H or Ac;
including enantiomers, scalemic and racemic mixtures, and pharmaceutically acceptable salts thereof.

The application further provides a compound prepared by the above process, wherein the compound is selected from the group consisting of:

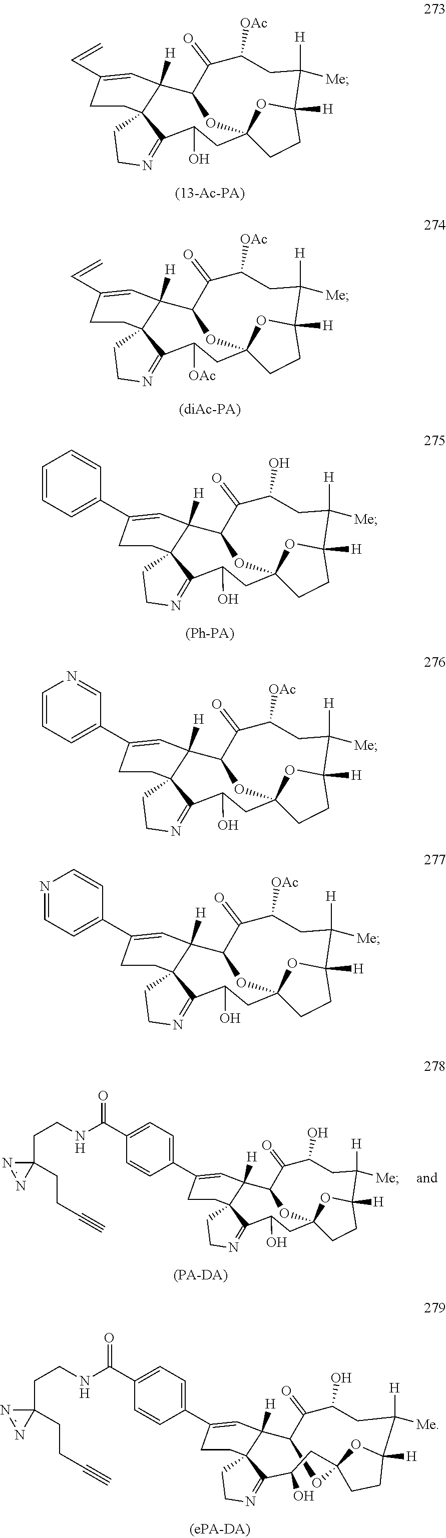

The application provides a process of preparing diverse analogues of Portimine A, comprising the following steps:

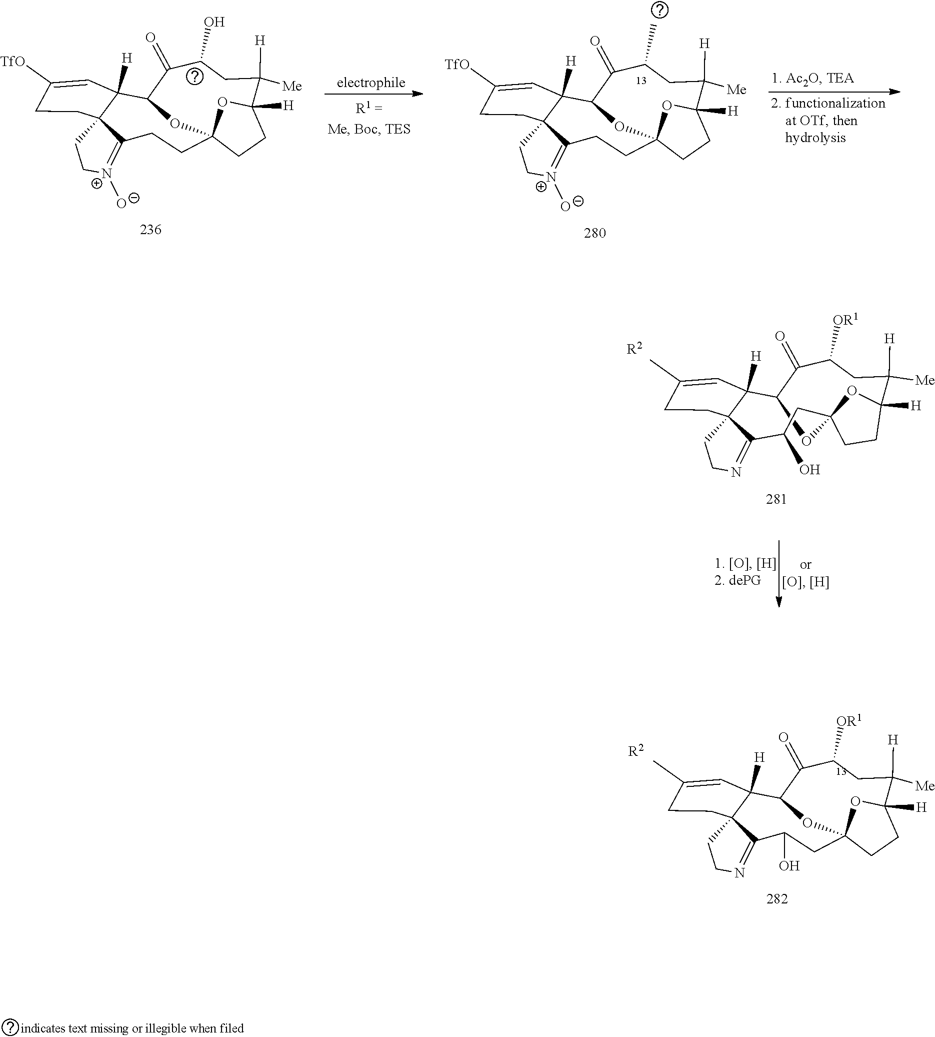

wherein:
R¹ is H, Me, Boc, or TES; and
R² is H, halo, (C₆-C₁₀) aryl, (C₂-C₆) alkenyl, or (C₅—Cl₂) heteroaryl, wherein each aryl is optionally substituted with —NH(CH₂)₂C(—N═N—)(CH₂)₂(C≡CH).
R′ is H or Ac;
including enantiomers, scalemic and racemic mixtures, and pharmaceutically acceptable salts thereof.

The application further provides a compound prepared by the above process, wherein the compound is selected from the group consisting of:

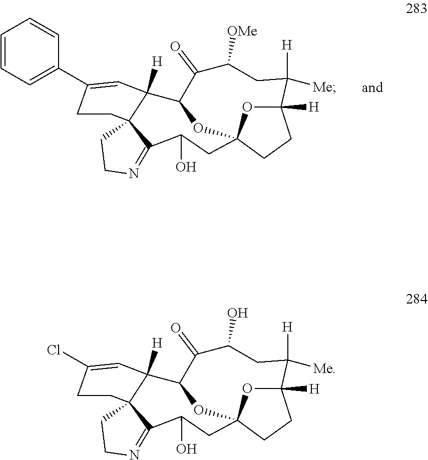

The application provides a process of preparing diverse analogues of Portimine A, comprising the following steps:

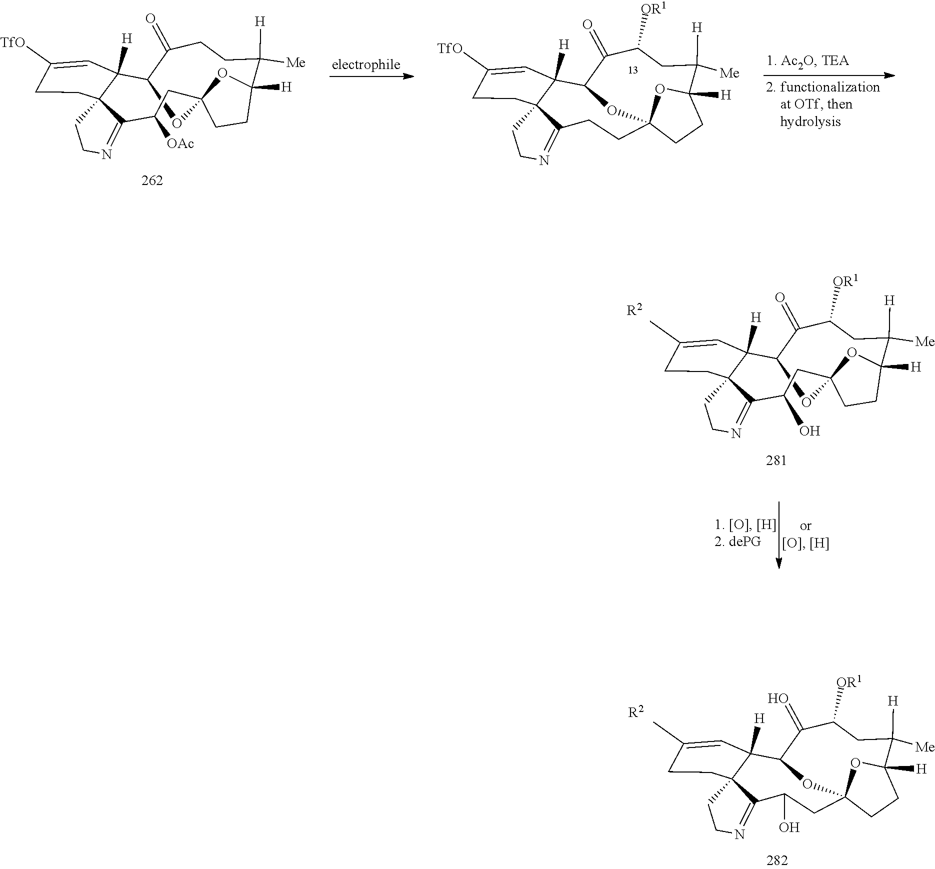

wherein:
R¹ is H, Me, Boc, or TES; and
R² is H, halo, (C₆-C₁₀) aryl, (C₂-C₆) alkenyl, or (C₅—Cl₂) heteroaryl, wherein each aryl is optionally substituted with —NH(CH₂)₂C(—N═N—)(CH₂)₂(C≡CH).
R′ is H or Ac;
including enantiomers, scalemic and racemic mixtures, and pharmaceutically acceptable salts thereof.

The application further provides a compound prepared by the above process, wherein the compound is selected from the group consisting of:

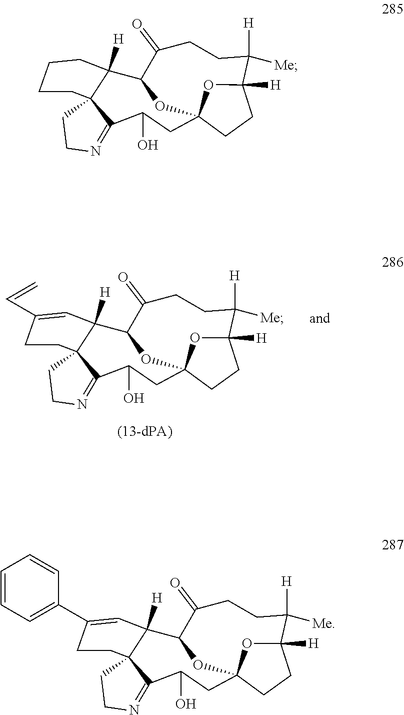

The application provides a process of any one of the above embodiments, wherein the Portimine A analogue product is useful in forming an antibody-drug conjugate (ADC) for the treatment of cancer.

The application provides a method of forming antibody-drug conjugate (ADC) for the treatment of cancer, comprising use of the compound of any one of the above embodiments.

The application provides a method of treating cancer, comprising administering to a patient in need thereof a pharmaceutically effective amount of any one of the above embodiments capable of forming antibody-drug conjugate (ADC).

### DETAILED DESCRIPTION OF THE INVENTION

By analogy to the logic of two-phase synthesis, a minimally oxidized macrocyclic intermediate to the portimines was targeted with the assumption that a proper choreography of oxidation events would solve both connectivity and stereochemical issues. The only C—O bonds to be installed at the outset were those residing at C-4 (imine carbon) and C-10 (secondary alcohol). A triple bond on C-7/8 would be a surrogate for the eventual C-7 oxidation and, critically, offer a strategic disconnection to the macrocycle using ring-closing alkyne metathesis (RCAM).³¹ Such a tactic would thereby minimize unstable functional groups and redundant redox manipulations since four key oxidations (C-3, C-13, C-14, C-15) would occur post-macrocyclization. To minimize protecting groups (PGs), the innate reactivity and conformational preferences are utilized via ring-chain reorganization/reconstitution steps. Upon unraveling the macrocycle, the dialkynated precursor (7) traces back to accessible building blocks (FIG. 1B).

Finally, in order to maximize access to useful analogs, an unusually stable vinyl triflate was selected as a key functionality to be carried through the entire synthesis. The simplicity and scalability of this route would ultimately pave the way to unravel the mechanism of action of 1 and 2 and fully evaluate its therapeutic potential.

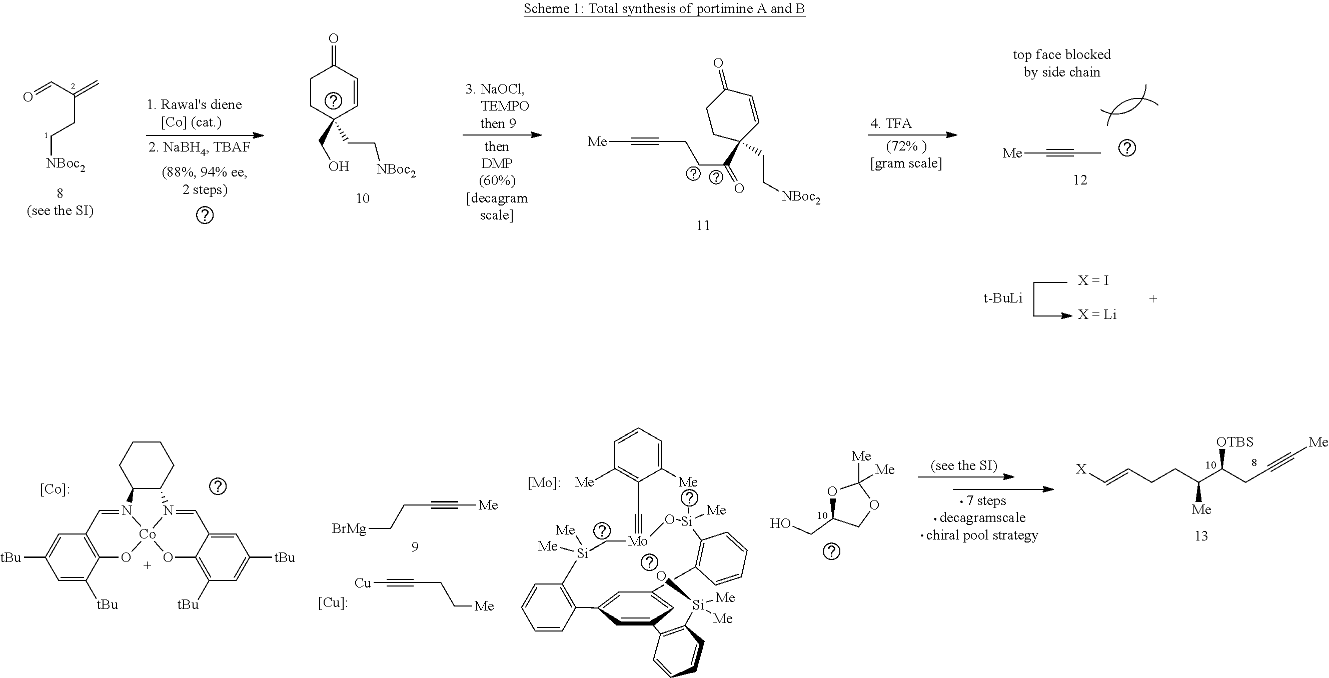

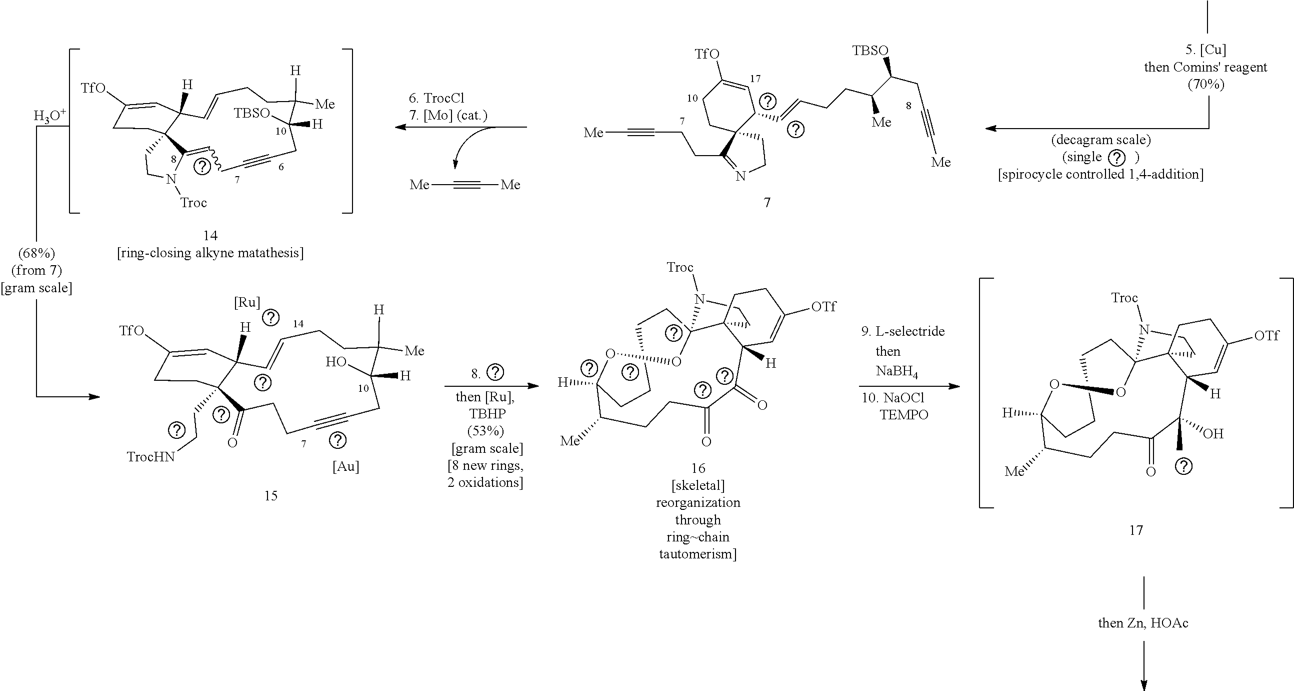

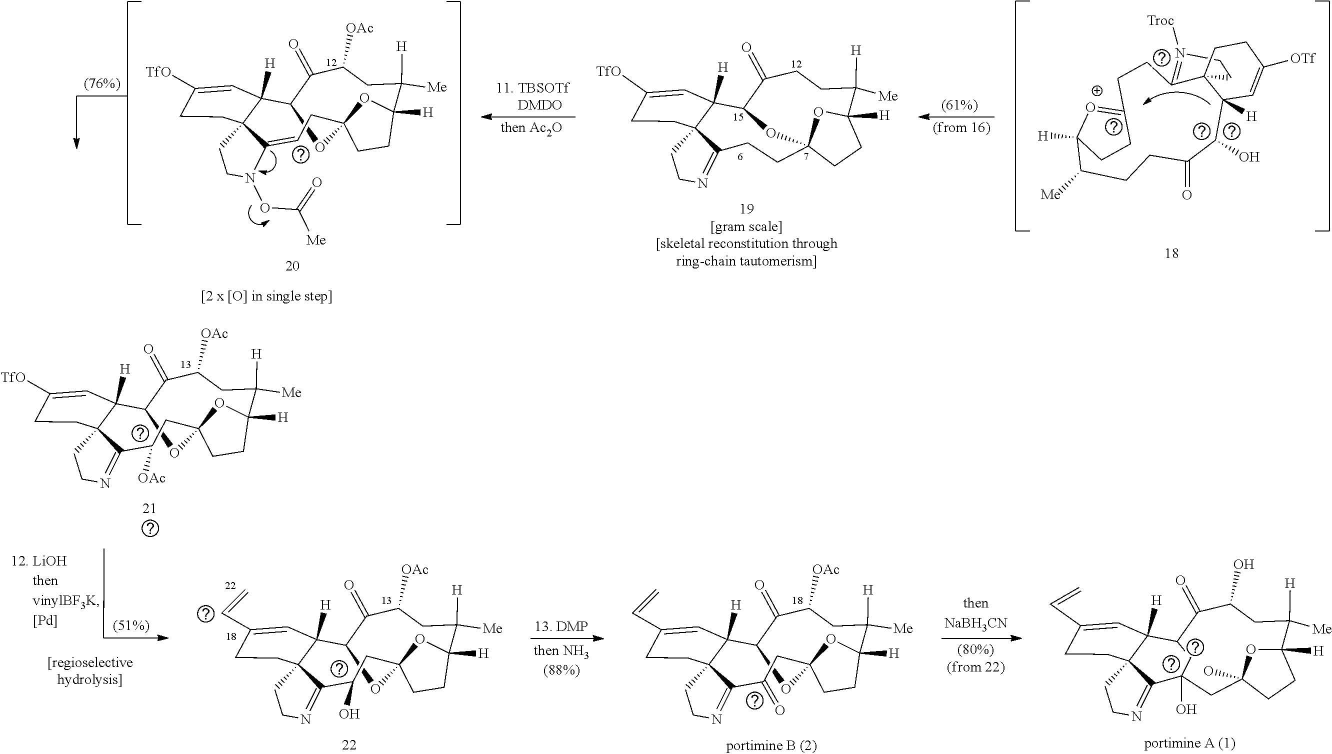

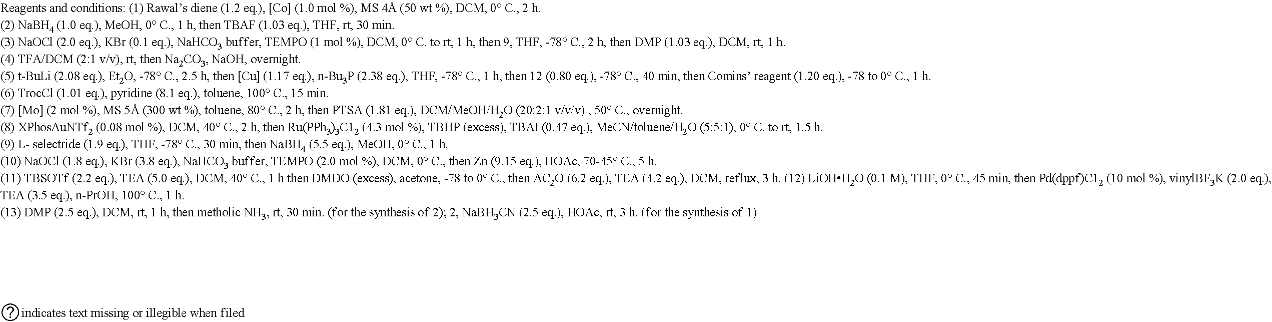

The synthesis is outlined in Scheme 1 and commences with a scalable, asymmetric Diels-Alder cycloaddition developed by Rawal et al., which established the C-3 chirality. Reduction of C-4, followed by removing the carbamate auxiliary with TBAF, afforded 10 in 88% yield, 94% ee on 50-gram scale. A non-strategic reduction of the C-4 aldehyde was required due to its instability in the elimination step (see SI, buffered TBAF, HF, HCl, CsF, etc. all resulted in decomposition). The requisite methyl-capped alkyne sidechain can be installed via a sequence including Grignard addition and two oxidations, delivering 11 as the final product in 60% yield after a single purification. Treatment of 11 with TFA in DCM afforded spirocyclic imine 12 in 72% yield on gram scale.

The synthesis of fragment 13 was carried out from inexpensive (S)-solketal (ca. $1.1/g, see Scheme 2 for details). To affix this subunit onto the established chiral spirocyclic core 12, a stereoselective copper-mediated conjugate addition was applied. The choice of copper (I) reagent was crucial since switching to other common copper (I) salts, such as CuI, CuCN, and CuBr, showed little to no observable conversion. To promote the nucleophilicity and increase the solubility of [Cu], n-Bu₃P was used as the ligand. The stereochemical outcome in this step is controlled by the intrinsic configuration of the spiro-cycle, wherein the side chain blocked the top face. Direct treatment of the in situ generated enolate with Comins' reagent ensured the correct regiochemical olefin outcome and smoothly delivered vinyl triflate 7 as a single product (6.5-g scale), which possessed all skeletal carbon atoms required for 1 and 2.

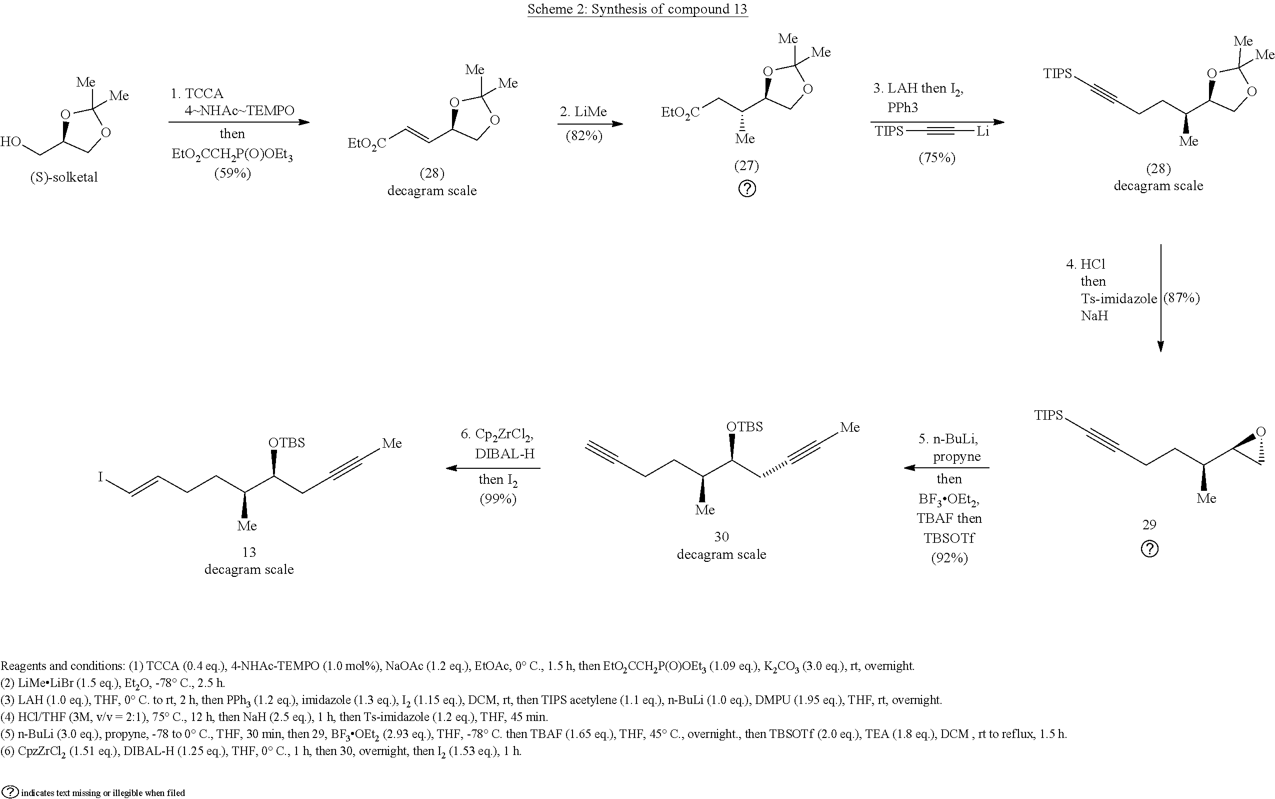

Attention was then turned to constructing the 14-membered macrocycle in portimines' skeleton through RCAM, a maneuver that might be derailed by the imine, olefins, or vinyl triflate. FHrstner's extremely efficient, canopy-shaped catalyst, [Mo] was chosen at this point due its outstanding functional compatibility and demonstrated robustness.³⁵ The pivotal RCAM step could indeed be achieved when heated 7 with [Mo] in toluene in 53-65% yield. Presumably due to the aforementioned functional groups, especially the imine nitrogen,³⁶ a relatively high catalyst loading (12.5 mol % to 15 mol %), as well as pro-longed reaction time was required. To solve this problem, the imine was masked with Troc group, followed by exposing the formed enamide to 2.0 mol % [Mo]. In this case, formation of macrocycle 14 was complete in one hour. Treatment of crude 14 with acidic wet methanol liberated the C-4 ketone and deprotected the TBS ether on C-10,37 affording 15 as a white powder (68% overall isolated yield over two steps, gram scale).

Arrival at macrocycle 15 (the end of the “cyclase phase”) was a milestone since all requisite core C≡C bonds were in place to arrive at 1 and 2. All that remained was installation of five oxygen atoms at C-5, C-7, C-13, C-14, and C-15. The “oxidase-phase” commenced with oxidation of C-14/15 since olefin-oxidations are known to be compatible with alkynes. Initial attempts included the use oxidants (i.e., Os04, m-CPBA, etc.) that would result in a net two-electron outcome (epoxide or diol). Unfortunately, the stereochemical outcome of C-15 in all cases was undesired. Therefore, a net six-electron oxidation catalyzed by ruthenium was chosen to arrive at a diketone,³⁸ with an eventual strategic reduction to set the desired stereochemistry. Stronger oxidants of this type, however, will not tolerate the presence of an alkyne. For this purpose, an internal protection strategy was designed. This goal was achieved upon skeletal reorganization by refluxing 15 and XPhosAuNTf2 (0.8 mol %) in DCM. The newly-formed tricyclic system organized all potentially sensitive sites (nitrogen on C-1, C-4 ketone, C-7-C-8 alkyne, and C-10 alcohol) into their in-active states with a newly rigidified backbone to control downstream stereochemical issues. Subsequent six-electron oxidation under Ru-catalyst led to diketone 16 in a 53% isolated yield (gram-scale) thereby minimizing reliance on protecting groups.

With this newly constructed rigid polycyclic system in place, the correct oxidation state and stereochemistry on C-14 and C-15 were installed. Site-specific reduction of C-14 was accomplished using L-selectride, followed by treating the crude material with sodium borohydride (NaBH4) to afford a diol, which possessed the desired stereochemistry on C-15. Subsequently, C-14 was selectively returned to the ketone oxidation state with TEMPO/NaOCl. Other oxidants (DMP, IBX, activated DMSO, TPAP/NMO, etc.) tested for this step showed poor selectivity. Remarkably, upon heating crude 17 with zinc powder in acetic acid, the Troc group was dismantled and the polycyclic ring system spontaneously unraveled through ring-chain tautomerization (via 18) to liberate the dideoxyportimine triflate 19. The yield of this two-step sequence was 48%.

Only two oxygen atoms, at C-13 and C-15, remained to be installed to complete the synthesis. Both of the oxidations could be achieved in a single step by treating the silyl enol ether of 19 with DMDO to afford a nitrone intermediate.³⁹ Subsequent heating of the crude nitrone in the presence of Ac₂O and TEA presumably forms N-oxyenamine 20, that spontaneously undergoes Boekelheide type rearrangement to deliver diacetate 21 as a single diastereomer in 76% yield on 90-mg scale.⁴⁰⁻⁴² The preferential cis-configuration of the C-4-C-5 double bond in 20 can explain the high stereochemical control. At this point, the vinyl triflate which had remained a silent observer throughout the synthesis was now called upon to append the final two carbon atoms of 1 and 2. Thus, selective hydrolysis of the C-5 acetate using LiOH, followed by Suzuki coupling to install the exocyclic vinyl group delivered diene 22 in 51% yield.⁴³ To complete the synthesis, 22 could be oxidized with DMP, followed by ammonia, affording portimine B (2) in good yield (88%). During these studies it was suspected that the originally assigned structure of 2 as a ring-opened tautomer was incorrect and this was now confirmed to be the same ring-closed tautomer expressed in 1. To complete the synthesis of 1, crude 2 in solution could be chemoselectively reduced (NaBH3CN) in high isolated yield (80%) on the heavily oxygenated natural product itself.

### REFERENCES

(1) Stivala, C. E.; Benoit, E.; Arioz, R.; Servent, D.; Novikov, A.; Molgó, J.; Zakarian, A. Synthesis and Biology of Cyclic Imine Toxins, an Emerging Class of Potent, Globally Distributed Marine Toxins. Nat. Prod. Rep. 2015, 32, 411-435.
(2) Molgó, J.; Marchot, P.; Aráoz, R.; Benoit, E.; Iorga, B. I.; Zakarian, A.; Taylor, P.; Bourne, Y.; Servent, D. Cyclic Imine Toxins from Dinoflagellates: A Growing Family of Potent Antagonists of the Nicotinic Acetylcholine Receptors. J. Neurochem. 2017, 142, 41-51.
(3) Munday, R.; Selwood, A. I.; Rhodes, L. Acute Toxicity of Pinnatoxins E, F and G to Mice. Toxicon 2012, 60, 995-999.
(4) Munday, R.; Quilliam, M. A.; LeBlanc, P.; Lewis, N.; Gallant, P.; Sperker, S. A.; Ewart, H. S.; MacKinnon, S. L. Investigations into the Toxicology of Spirolides, a Group of Marine Phycotoxins. Toxins 2011, 4, 1-14.
(5) Munday, R.; Towers, N. R.; Mackenzie, L.; Beuzenberg, V.; Holland, P. T.; Miles, C. O. Acute Toxicity of Gymnodimine to Mice. Toxicon 2004, 44, 173-178.
(6) Selwood, A. I.; Wilkins, A. L.; Munday, R.; Shi, F.; Rhodes, L. L.; Holland, P. T. Portimine: A Bioactive Metabolite from the Benthic Dinoflagellate Vulcanodinium Rugosum. Tetrahedron Lett. 2013, 54, 4705-4707.
(7) Fribley, A. M.; Xi, Y.; Makris, C.; Alves-de-Souza, C.; York, R.; Tomas, C.; Wright, J. L.; Strangman, W. K. Identification of Portimine B, a New Cell Permeable Spiroimine That Induces Apoptosis in Oral Squamous Cell Carcinoma. ACS Med. Chem. Lett. 2018, 10, 175-179.
(8) Hermawan, I.; Higa, M.; Hutabarat, P. U.; Fujiwara, T.; Akiyama, K.; Kanamoto, A.; Haruyama, T.; Kobayashi, N.; Higashi, M.; Suda, S.; Tanaka, J. Kabirimine, a New Cyclic Imine from an Okinawan Dinoflagellate. Mar. Drugs 2019, 17, 353.
(9) Cuddihy, S. L.; Drake, S.; Harwood, D. T.; Selwood, A. I.; McNabb, P. S.; Hampton, M. B. The Marine Cytotoxin Portimine Is a Potent and Selective Inducer of Apoptosis. Apoptosis 2016, 21, 1447-1452.
(10) Brooke, D. G.; Cervin, G.; Champeau, O.; Harwood, D. T.; Pavia, H.; Selwood, A. I.; Svenson, J.; Tremblay, L. A.; Cahill, P. L. Antifouling activity of portimine, select semisynthetic analogues, and other microalga-derived spirocyclic imines. Biofouling 2018, 34, 950-961.
(11) Izumida, M.; Suga, K.; Ishibashi, F.; Kubo, Y. The Spirocyclic Imine from a Marine Benthic Dinoflagellate, Portimine, Is a Potent Anti-Human Immunodeficiency Virus Type 1 Therapeutic Lead Compound. Mar. Drugs 2019, 17, 495.
(12) Hogeveen, K.; Huet, S.; Besnard, C.; Murray, J. S.; Harwood, D. T.; Selwood, A. I.; Fessard, V. In vitro Investigation of the Genotoxicity of Portimine, a Cyclic Imine Toxin Produced by the Dinoflagellate Vulcanodinium Rugosum, on Human Hepatic HepaRG Cells. Toxicology in Vitro 2021, 73, 105125.
(13) MacKinnon, S. L.; Cembella, A. D.; Burton, I. W.; Lewis, N.; LeBlanc, P.; Walter, J. A. Biosynthesis of 13-Desmethyl Spirolide C by the Dinoflagellate Alexandrium ostenfeldii. J. Org. Chem. 2006, 71, 8724-8731.
(14) Kellmann, R.; Stuken, A.; Orr, R. J.; Svendsen, H. M.; Jakobsen, K. S. Biosynthesis and Molecular Genetics of Polyketides in Marine Dinoflagellates. Mar. Drugs 2010, 8, 1011-1048.
(15) Van Wagoner, R. M.; Satake, M.; Wright, J. L. Polyketide Biosynthesis in Dinoflagellates: What Makes It Different? Nat. Prod. Rep. 2014, 31, 1101-1137.
(16) McCauley, J. A.; Nagasawa, K.; Lander, P. A.; Mischke, S. G.; Semones, M. A.; Kishi, Y. Total Synthesis of Pinnatoxin A. J. Am. Chem. Soc. 1998, 120, 7647-7648.
(17) Stivala, C. E.; Zakarian, A. Total Synthesis of (+)-Pinnatoxin A. J. Am. Chem. Soc. 2008, 130, 3774-3776.
(18) Nakamura, S.; Kikuchi, F.; Hashimoto, S. Total Synthesis of Pinnatoxin A. Angew. Chem., Int. Ed. 2008, 47, 7091-7094.
(19) Araoz, R.; Servent, D.; Molgó, J.; Iorga, B. I.; Fruchart-Gaillard, C.; Benoit, E.; Gu, Z.; Stivala, C.; Zakarian, A. Total Synthesis of Pinnatoxins A and G and Revision of the Mode of Action of Pinnatoxin a. J. Am. Chem. Soc. 2011, 133, 10499-10511.
(20) Kong, K.; Moussa, Z.; Lee, C.; Romo, D. Total Synthesis of the Spirocyclic Imine Marine Toxin (−)-Gymnodimine and an Unnatural C4-Epimer. J. Am. Chem. Soc. 2011, 133, 19844-19856.
(21) Aitken, H. R. M.; Brimble, M. A.; Furkert, D. P. A catalytic asymmetric ene reaction for direct preparation of α-hydroxy 1, 4-diketones as intermediates in natural product synthesis. Synlett 2020, 31, 687-690.
(22) Ding, X.-B.; Aitken, H. R.; Pearl, E. S.; Furkert, D. P.; Brimble, M. A. Synthesis of the C4-C16 Polyketide Fragment of Portimines A and B. J. Org. Chem. 2021, 86, 12840-12850.
(23) Li, L.; El Khoury, A.; Clement, B. O. N.; Wu, C.; Harran, P. G. Asymmetric Organocatalysis Enables Rapid Assembly of Portimine Precursor Chains. Org. Lett. 2022, 24, 2607-2612.
(24) Saito, T.; Fujiwara, K.; Kondo, Y.; Akiba, U.; Suzuki, T. Synthesis of the cyclohexene segment of portimine. Tetrahedron Lett. 2019, 60, 386-389.
(25) Chen, K.; Baran, P. S. Nature 2009 459, 824-828..
(26) Jorgensen, L.; McKerrall, S. J.; Kuttruff, C. A.; Ungeheuer, F.; Felding, J.; Baran, P. S. 14-Step Synthesis of (+)-Ingenol from (+)-3-Carene. Science 2013, 341, 878-882.
(27) Wilde, N. C.; Isomura, M.; Mendoza, A.; Baran, P. S. Two-Phase Synthesis of (−)-Taxuyunnanine D. J. Am. Chem. Soc. 2014, 136, 4909-4912.
(28) Kawamura, S.; Chu, H.; Felding, J.; Baran, P. S. Nineteen-step total synthesis of (+)-phorbol, Nature, 2016, 532, 90-93.
(29) Chu, H.; Smith, J. M.; Felding, J.; Baran, P. S. Scalable Synthesis of (−)-Thapsigargin, ACS Cent. Sci. 2017, 3, 47-51
(30) Kanda, Y.; Nakamura, H.; Umemiya, S.; Puthukanoori, R. K.; Murthy Appala, V. R.; Gaddamanugu, G. K.; Paraselli, B. R.; Baran, P. S. Two-Phase Synthesis of Taxol. J Am. Chem. Soc. 2020, 142, 10526-10533.
(31) Furstner, A. Alkyne Metathesis on the Rise. Angew. Chem., Int. Ed. 2013, 52, 2794-2819.
(32) Huang, Y.; Iwama, T.; Rawal, V. H. Design and Development of Highly Effective Lewis Acid Catalysts for Enantioselective Diels-Alder Reactions. J. Am. Chem. Soc. 2002, 124, 5950-5951.
(33) Corey, E. J.; Beames, D. J. Mixed Cuprate Reagents of Type R₁R₂CuLi Which Allow Selective Group Transfer. J. Am. Chem. Soc. 1972, 94, 7210-7211.
(34) Coates, G. E.; Parkin, C. Ethynyl-Copper(I) Complexes with Tertiary Phosphines. J Inorg. Nucl. Chem. 1961, 22, 59-67.
(35) Hillenbrand, J.; Leutzsch, M., Yiannakas, E.; Gordon, C. P.; Wille, C.; Nöthling, N.; Copéret, C.; Furstner, A. “Canopy Catalysts” for Alkyne Metathesis: Molybdenum Alkylidyne Complexes with a Tripodal Ligand Framework. J Am. Chem. Soc. 2020, 142, 11279-11294.
(36) Furstner, A. The Ascent of Alkyne Metathesis to Strategy-Level Status. J. Am. Chem. Soc. 2021, 143, 15538-15555.
(37) Wang, Z.; Krogsgaard-Larsen, N.; Daniels, B.; Furkert, D. P.; Brimble, M. A. Cyclic Enecarbamates as Precursors of α,β-Unsaturated Iminium Ions: Reactivity and Synthesis of 6,6-Spirocyclic Ring Systems. J Org. Chem. 2016, 81, 10366-10375.
(38) Chen, S.; Liu, Z.; Shi, E.; Chen, L.; Wei, W.; Li, H.; Cheng, Y.; Wan, X. Ruthenium-Catalyzed Oxidation of Alkenes at Room Temperature: A Practical and Concise Approach to α-Diketones. Org. Lett. 2011, 13, 2274-2277.
(39) Boyd, D. R.; Coulter, P. B.; McGuckin, M. R.; Sharma, N. D.; Jennings, W. B.; Wilson, V. E. Imines and Derivatives. Part 24. Nitrone Synthesis by Imine Oxidation Using Either a Peroxyacid or Dimethyldioxirane. J. Chem. Soc., Perkin Trans. 1 1990, 301.
(40) Cummins, C. H.; Coates, R. M. α-Oxygenation of Aldehydes and Cyclic Ketones by Acylation-Rearrangement of Nitrones. J. Org. Chem. 1983, 48, 2070-2076.
(41) Beshara, C. S.; Hall, A.; Jenkins, R. L.; Jones, K. L.; Jones, T. C.; Killeen, N. M.; Taylor, P. H.; Thomas, S. P.; Tomkinson, N. C. A General Method for the α-Acyloxylation of Carbonyl Compounds. Org. Lett. 2005, 7, 5729-5732.
(42) Bartlett, S.; Keiter, K.; Zavesky, B.; Johnson, J. Formation of Complex α-Imino Esters via Multihetero-Cope Rearrangement of α-Keto Ester Derived Nitrones. Synthesis 2018, 51, 203-212.
(43) Molander, G. A.; Rivero, M. R. Suzuki Cross-Coupling Reactions of Potassium Alkenyltrifluoroborates. Org. Lett. 2001, 4, 107-109.

### DETAILED DESCRIPTION OF THE FIGURES

FIG. 1. Portimine A is non-toxic to primary human PBMCs. Portimine A and its functional analogs show stereo-selective acute toxicity in cancer cells. (A) PA and Ph-PA have similar cell toxicity in breast (HCC1806) and leukemia (Jurkat) cancer cell lines after 36-hour treatment while PB and ePA show minimal effects. (B) Structure of portimine related analogs: phenyl-portimine A (Ph-PA, 36) and epi-portimine A (ePA, 38). Structure of fully functionalized portimine A-based epimeric probe pair: portimine A-diazirine-alkyne (PA-DA, 35-2) and epi-portimine A-diazirine-alkyne (ePA-DA, 35-1). (C) Cell viability analysis of portimine A and analogs. Jurkat cells were treated with increasing concentrations of portimine A and analogs for 24 hours. All presented data as mean of biological replicated experiments (n=3). (D) Immunoblot of caspase 3 shows PA, PA-DA, Ph-PA induce cell apoptosis, while ePA, ePA-DA and PB do not. Jurkat cells were treated with portimine A or analogs for 12 hours at 10 nM.

PA and Ph-PA have ~2 nM IC50 to Jurkat cells after 12 hr. PA and Ph-PA substantially less toxic to healthy PBMCs compared to cancer T cell line Jurkat. Structurally related negative control ePA and PB are non-toxic in all cell types in the current concentrate range. PBMCs (25k per well), Jurkat (10k per well). Compound concentration was increased from 0.23 pM to 1 uM, 4× per gradient, PBMCs using primary B cell media from Wesley (heat deactivated), and Jurkat using normal RPMI media and incubated for 12 hr.

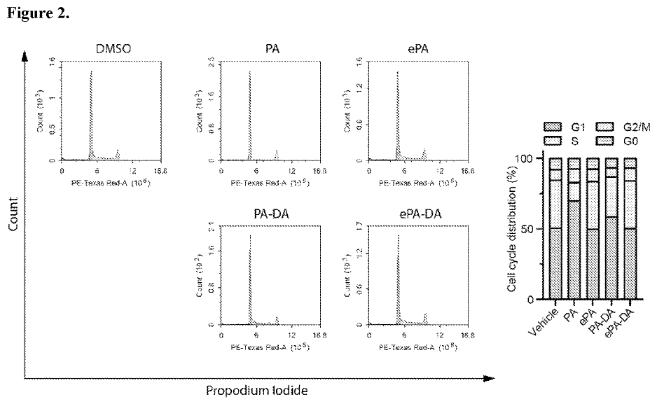

FIG. 2. Washout experiment reveals PA has acute cytotoxicity. Portimine A triggers cell cycle arrest in Jurkat cells. Cell cycle analysis in Jurkat cells. Jurkat cells were treated with 10 nM of indicated compounds for 24 hours, and propodium iodide was used to identify different stages of cells by flow cytometry. Treatment condition: Jurkat (50 k per well); Portimine A concentration: 0.007 nM-480 nM; Washout time point: 0.5, 2, 4, 6, 12, 24 hour, cells were washed once and suspended in PA-free RPMI media; In total treat cell for 36 hours; Measure cell viability with Cell Titerglo assay; Observe exposure-dependent decrease in IC50; Removing compound after 30 min results in ~5-fold decrease in IC50; IC50 after 30 min exposure ~5 nM; 4 hr ~2 nM; Overall, suggests fast acting cytotoxicity mechanism.

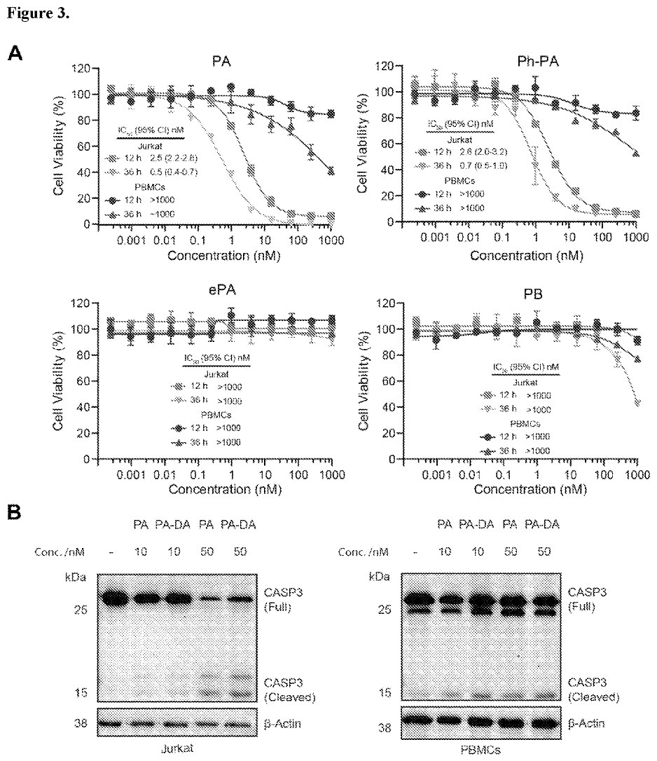

FIG. 3. Initial SAR studies: Assessing activity of PAL functionalized PTMA analogs. Portimine A and analogs do not induce apoptosis or affect cell viability in freshly isolated human PBMCs. (A) PA and Ph-PA show less toxic to healthy PBMCs compared to Jurkat cells. Both ePA and PB showed non-toxicity to both cells in the pM-nM concentration range. (B) Immunoblot of caspase-3 in Jurkat and PBMCs with indicated conditions. Both PA and PA-DA induced caspase-3 cleavage in a dose-dependent manner in Jurkat cells, but not human PBMCs. PTMA-DA found to have analogous activity to PTMA→Suitable target ID probe; epi-PTMA-DA and epi-PTMA-Ac-DA has no activity in the concentrate range, same as epi-PTMA→useful inactive probe.

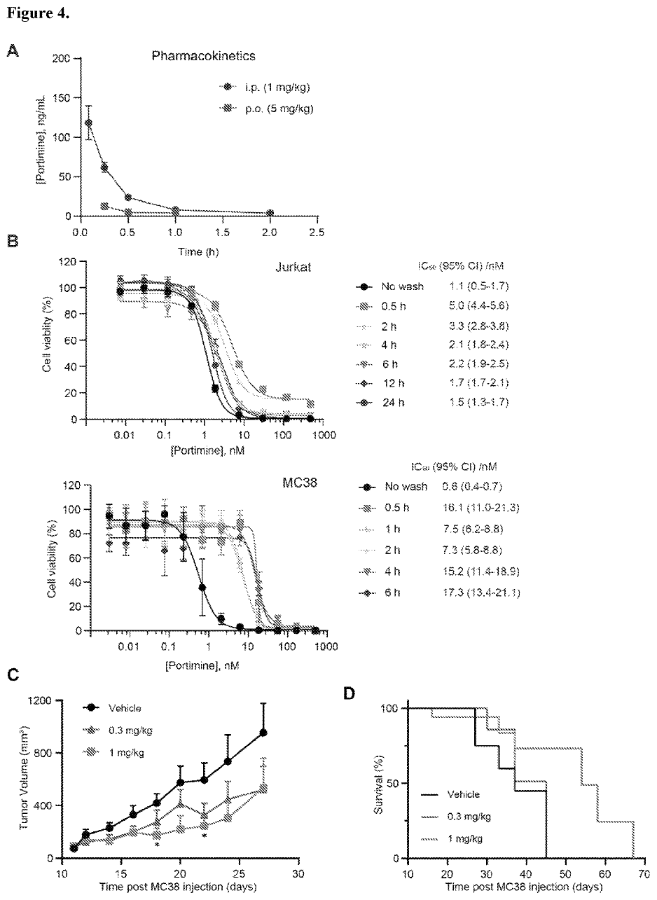

FIG. 4. Validation of NMD3 as target of Portimine A by Chemo-precipitation (ChP). Portimine A mouse pharmacokinetic properties and fast acting in vitro cell-based target engagement properties based on compound wash-out. (A) Pharmacokinetic studies of mouse intraperitoneal (i.p.) and oral (p.o.) administration for portimine (n=3). (B) Washout experiment performed in Jurkat and MC38 cells showed exposure-dependent decrease in ICso, reveals PA has a fast-acting cytotoxicity mechanism. Impact of PA [0.3 or 1 mg/kg intraperitoneally] on MC38 tumor growth in WT mice (n=6). (C) Kaplan-Myer survival curve of WT MC38 tumor-bearing mice (n=6) after treatment with PA as described in (D). Mice were euthanized when tumor area exceeded 2000 mm³. Statistical analysis was performed using ANOVA analysis followed by multiple comparisons test, * p≤0.05. Treatment condition: Jurkat (60 M per condition); PA-DA/ePA-DA concentration: 0.5 uM, PA or ePA: 5 uM; Incubate with probe with or without competitors (PA, ePA) for 30 min; UV irradiated for 20 min. (0.5 uM PA-DA add a no-UV condition); Standard pulldown & biotin-enrichment and western blot analysis (Elute condition: 2× sample buffer+500 uM biotin, boil for 20 mins); Primary antibody NMD3 (ProteinTech, 1:150), Actin (BioRad, 1:10000). Conclusion: NMD3 can be selectively binds by Portimine A; The interaction between NMD3 and Portimine A is non-covalent; Note -PA-DA binding to recombinantly overexpressed NMD3 in cells not observed, only to endogenous NMD3. Could suggest PA binds NMD3 only when in an endogenous complex.

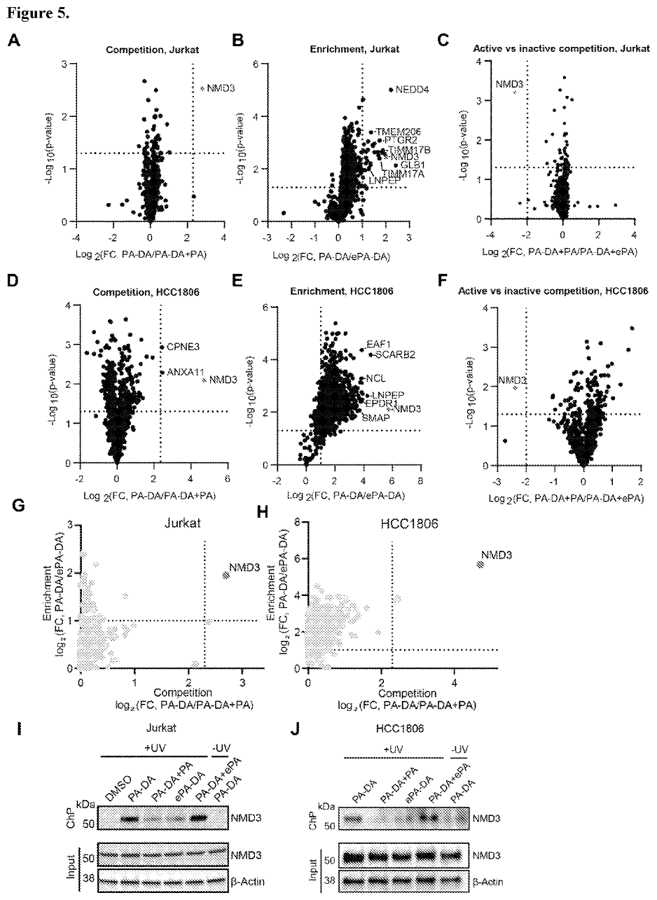

FIG. 5. PA related compounds induce cell apoptosis in Jurkats. Chemical proteomic analysis reveals NMD3 is the target of portimine A. Chemical proteomic profiling with PA-DA in Jurkat cells (A, B, C, G) and HCC1806 cells (D, E, F, H) show NMD3 is the primary target candidate of portimine A. Volcano plot in panels A and D show protein competition in cells treated with active photoaffinity probe PA-DA (500 nM) and DMSO or the active competitor portimine A (PA, 4 pM), dotted lines indicate threshold of fold change <4, p-value>0.05. Volcano plots in panels B and E show protein enrichment by active probe PA-DA over inactive probe ePA-DA (500 nM), dotted lines indicate threshold of fold change <2, p-value>0.05. Volcano plot in panels C and F show comparison between competition by active competitor compound PA to inactive control competitor ePA (4 M, PA-DA+PA/PA-DA+ePA), dotted lines indicate threshold of fold change>4, p-value>0.05. (I, J) Confirmation of PA-DA engagement of NMD3 in Jurkat and HCC1806 cells. UV-dependent labeling of endogenous NMD3 by PA-DA is blocked by PA, but not ePA, and was not substantially labeled by inactive probe ePA-DA or PA-DA with no UV-irradiation. Treatment condition: Jurkat (5M per well). Portimine A concentration: 2 nM-1000 nM; In total treat cell for 12 hours. Conclusion: PA causes cell apoptosis in dose-dependent manner; ePA does not show significant Caspase 3 activation at this concentration range.

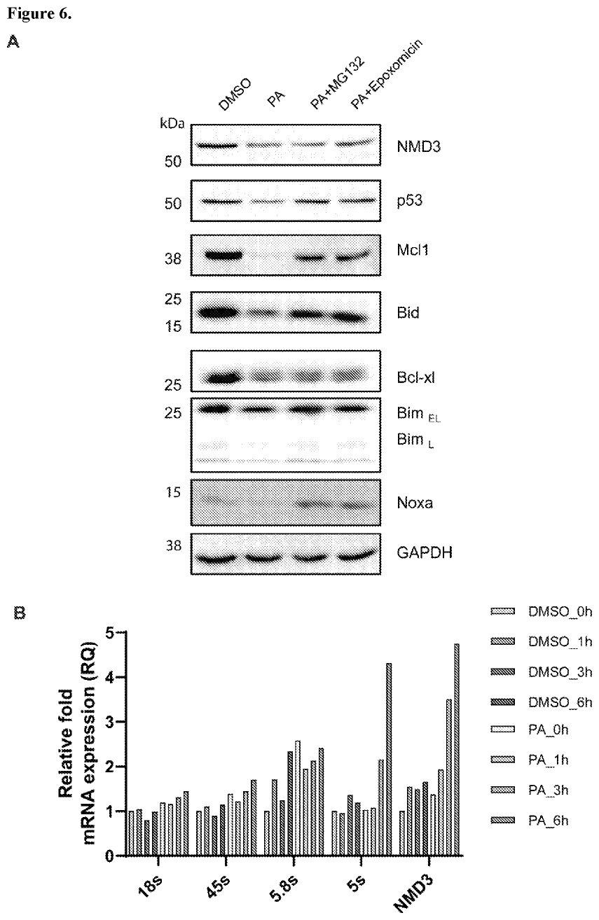

FIG. 6. Portimine A inhibits S phase entry in Jurkat cells. Portimine A downregulates NMD3 at protein level but upregulates in mRNA level in cells, as well as some essential apoptosis proteins. (A) NMD3 and Bcl-xl downregulated after PA treatment, and not by proteasome-mediated degradation. Protein degradation of p53, Mcl1, Bid, Bim, Noxa can be rescued by MG132 or Epoxomicin. (B) NMD3 mRNA increased after PA treatment, but not ePA control compound. Jurkat cells were treated with indicated compounds for 24 hr; Cells were then fixed and perform Propidium Iodide staining. Conclusion: S phase entry was inhibited in Jurkat cells with 10 nM treatment of PA/PADA; PA/PADA does not affect G2M/sub-G1 (mostly apoptotic cells).

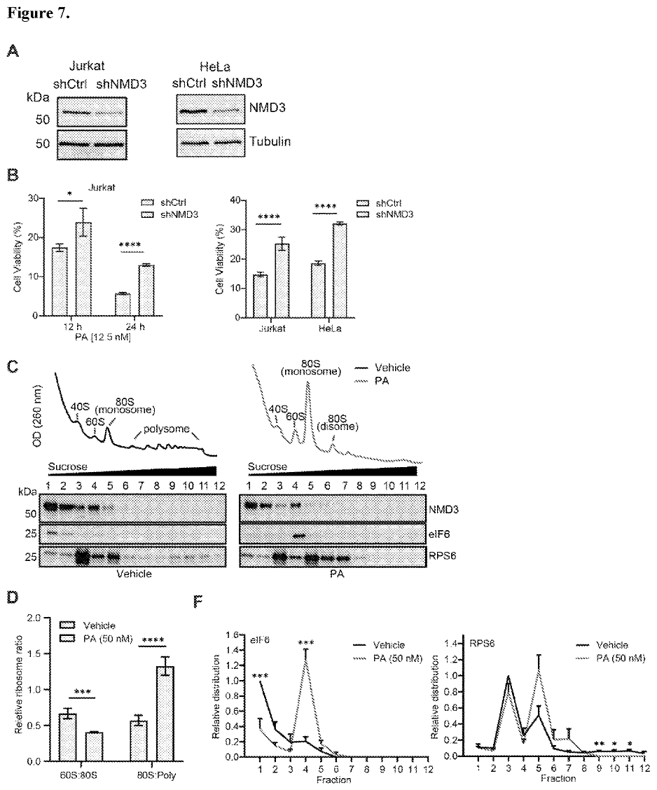

FIG. 7. Portimine A targets NMD3 and prevents polysome formation. NMD3 is downregulated by PA in Jurkat cells in a time- and dose-dependent manner. (A) Immunoblot showing reduced NMD3 levels in shNMD3 Jurkat and HeLa cells. (B) PA has less toxic effects in cells where NMD3 is knocked down (shNMD3) compared to shCtrl cells. Jurkat cells were treated with 12.5 nM of PA for 12 or 24 hours. Jurkat and HeLa cells were treated with 5 nM or 150 nM of PA for 24 hours. Data represents mean±SD of biologically replicated experiments (n=3). (C) Polysome profiling reveals the protein translation inhibition by portimine A. Jurkat cells were treated with DMSO or portimine A for 6 hours at 50 nM. Cell lysates were fractionated by sucrose gradient (5-50%) and each fraction were analyzed by western blot. eIF6 levels increase in 60S subunit fraction and decrease in ribosome-free fractions after PA treatment. RPS6, a 40S and polysome subunits marker, is decreased in polysome fractions after PA treatment. Results are representative of three independent experiments. (D) Quantifications of 60S:80S ratio and 80S:polysome ratio from polysome profiling assay as shown in FIG. 7C. (E) Quantification of relative protein distribution in fractions as displayed in FIG. 7C. Relative protein levels in each fraction were normalized to the peak fraction of the indicated protein from the DMSO cells and plotted. Data represents mean±SD of biologically replicated experiments (n=3). Statistical analysis was performed using multiple unpaired Student t-test. * p≤0.05; ** p≤0.01; *** p≤0.005; **** p≤0.0001. Treatment condition: Jurkat (5 M per condition); Incubate cells (1 M/mL) with inhibitors for 6 hr, 12 hr and 24 hr, respectively. Conclusion: NMD3 is downregulated by PA treatment at 10 nM at 12 hrs; KPT-330 downregulates NMD3 at higher (<50 nM) concentration at 24 hrs; XPO1 levels appear unaffected by PA; p53 can be downregulated by Portimine A treatment; Similar to KPT-330, PA does not affect Bcl2 levels at 6/12 hrs.

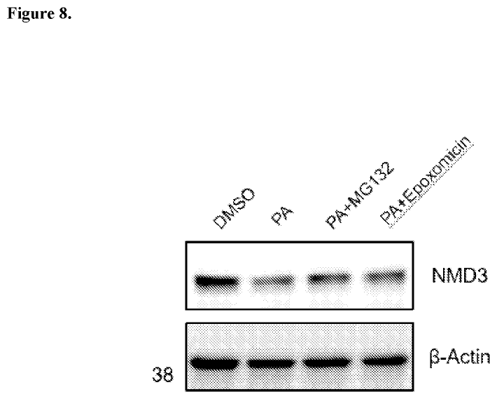

FIG. 8. NMD3 down regulation is not occurring through proteosome-mediated degradation. Treatment condition: Jurkat (10 M per condition); PA concentration: 10 nM; Preincubate cells (1 M/mL) with MG132 or Epoxomicin for 2 hr. Incubate cells with PA with or without MG132 or Epoxomicin for another 12 hr. Conclusion: NMD3 downregulation is not by proteasome-mediated degradation; Similar to XPO1 inhibitors, might be result of transcriptional inhibition.

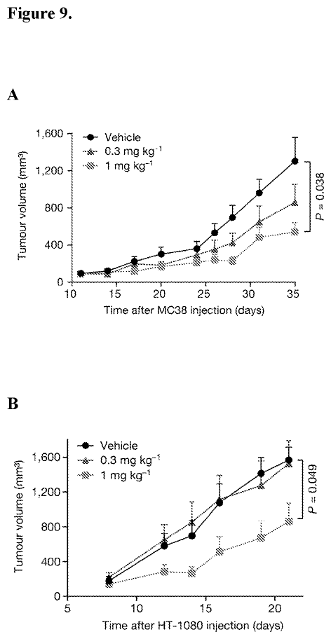

FIG. 9. Portimine A displays potent anti-tumor activity in human and syngeneic mouse tumor models. (A) Systemic Portimine A in MC38 Syngeneic Tumor Xenograft Model; and (B) Systemic Portimine A in HT-1080 Tumor Xenograft Model.

Preliminary cell-based target engagement studies, based on compound washout, indicates PA is fast acting with <1 hr of exposure resulting in minimal loss of potency. Cmax-driven pharmacology and facile synthetic access to material enabled the evaluation of PA in both a syngeneic (MC38 colon carcinoma) and a human metastatic mesenchymal (HT-1080 fibrosarcoma) mouse tumor xenograft model. Potent anti-tumor activity observed for PA at 0.3 or 1.0 mg/kg doses, following system administration of tolerated doses (i.p. delivery). Metabolic stability represents an opportunity for an additional IP position.

Results Indicate that Portimine a May Share Similar Anti-Cancer Effects as XPO1 Inhibitor KPT-330:

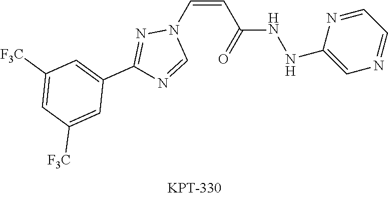 [SMILES: O=C(/C=C\n1cnc(-c2cc(C(F)(F)F)cc(C(F)(F)F)c2)n1)NNc1cnccn1]

i) NMD3 is the known adaptor of XPO1 and NMD3 appears required for XPO1 localization. No NMD3 inhibitor has been reported yet;
ii) KPT-330 is an FDA approved, first-in-class, oral Selective Inhibitor of Nuclear Export (SINE) compound, by covalently target XPO1 protein, IC50: 34-203 nM;
iii) XPO1 is frequently overexpressed and/or mutated in human cancers and functions as an oncogenic driver, XPO1-mediated export is increased in various cancers;
iv) KPT-330 induced rapid apoptosis at low nanomolar concentrations in a panel of 14 human T-cell acute lymphoblastic leukaemia (T-ALL) cell lines, with little toxicity to normal murine haematopoietic cells, similar to PA;
v) KPT-330 inhibits proliferation and induced cell cycle arrest in cancer cells, similar as PA; vi) KPT-330 downregulates XPO1 in cells by reduces mRNA expression and protein synthesis; and
vii) KPT-330 impeded nucleolar rRNA processing and reduced total levels of multiple mature rRNAs.

### General Experimental Conclusions:

i) NMD3 the only target emerging from chemoproteomic target ID studies in two cancer cell models;
ii) Endogenous NMD3 confirmed as a target of Portimine A and PA probe, but not inactive controls;
iii) NMD3 is thought to be a scaffolding protein for XPO1/CRM1, although not much is known about its precise roles;
iv) Portimine induces apoptosis and cell cycle arrest, similar to XPO1 inhibitors; and
v) Portimine A downregulates NMD3 protein levels. Likely not through degredation

### Embodiments

Embodiment 1. A process for preparing Portimine A (1),

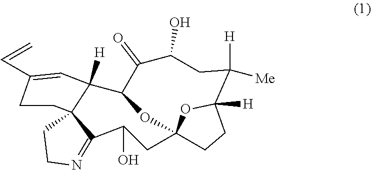

wherein the process comprises the Diels-Alder cycloaddition of Compound (8) to form Compound (10):

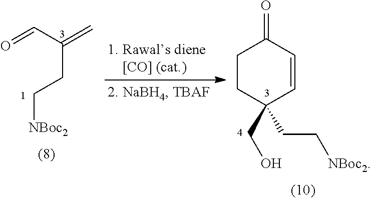

Embodiment 2. The process of Embodiment 1, further comprising the alcohol oxidation of Compound (10), subsequent Grignard addition, and Dess-Martin oxidation to form Compound (11):

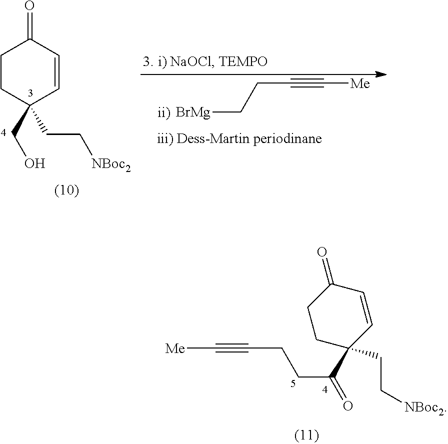

Embodiment 3. The process of Embodiment 2, further comprising the cyclization of Compound (11) to form Compound (12):

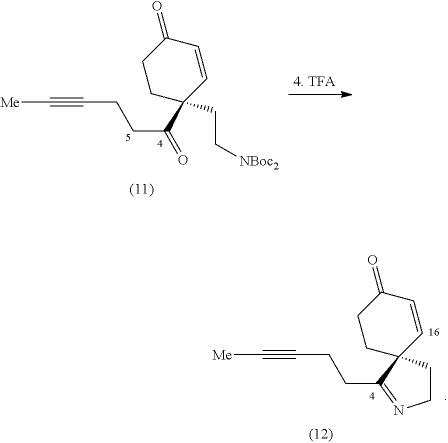

Embodiment 4. A process of preparing Compound (13) from (S)-solketal, comprising the following steps:

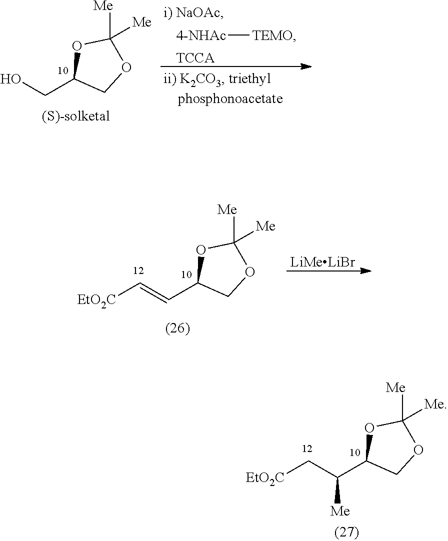

Embodiment 5. The process of Embodiment 4, further comprising the following steps:

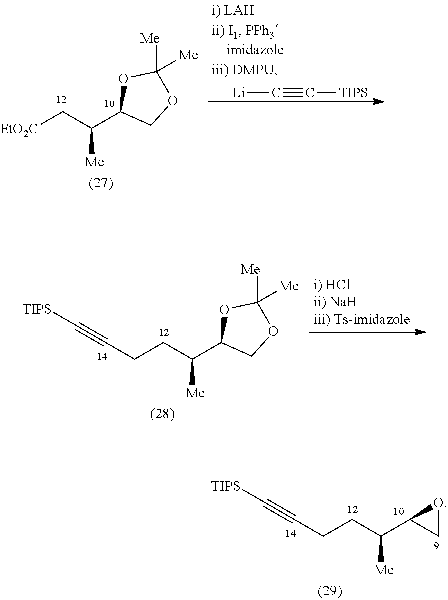

Embodiment 6. The process of Embodiment 5, further comprising the following steps:

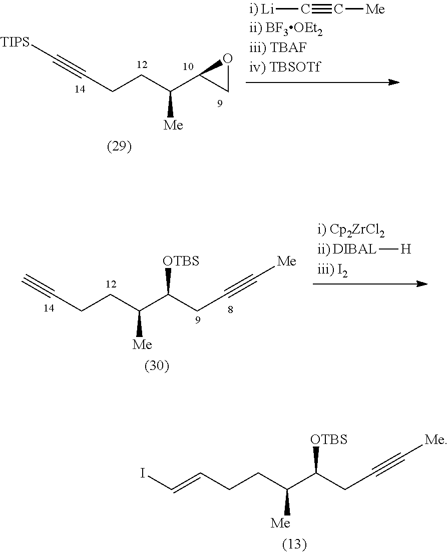

Embodiment 7. The process of Embodiment 3, further comprising the transmetallation of Compound (13) and conjugate addition to Compound (12) to form Compound (7):

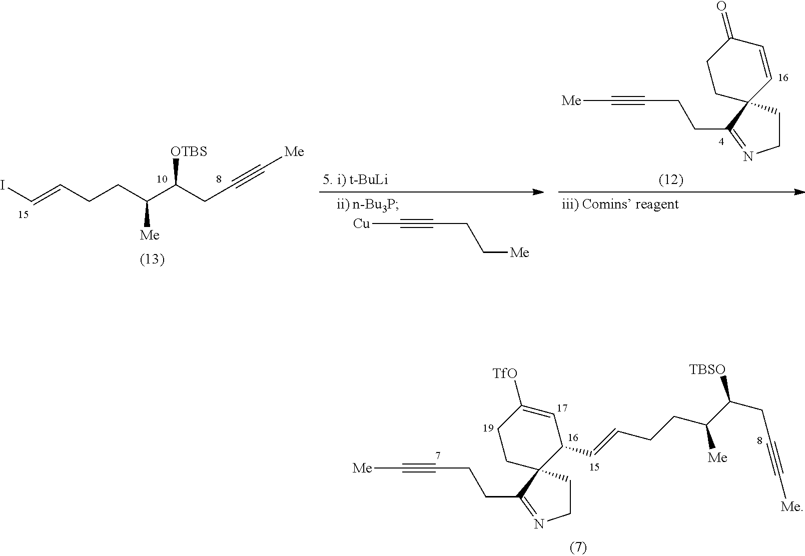

Embodiment 8. The process of Embodiment 7, further comprising the conversion of compound (7) to the N-protected imine Compound (31):

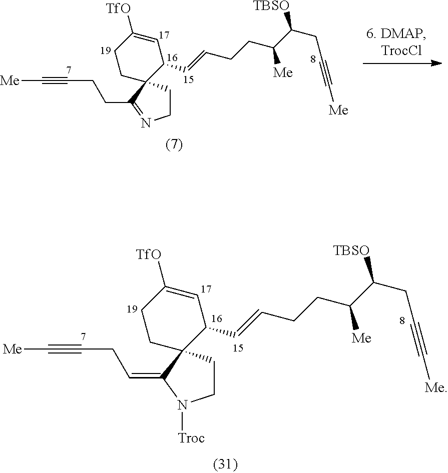

Embodiment 9. The process of Embodiment 8, further comprising Mo-catalyzed ring-closing alkyne metathesis to form Compound (15):

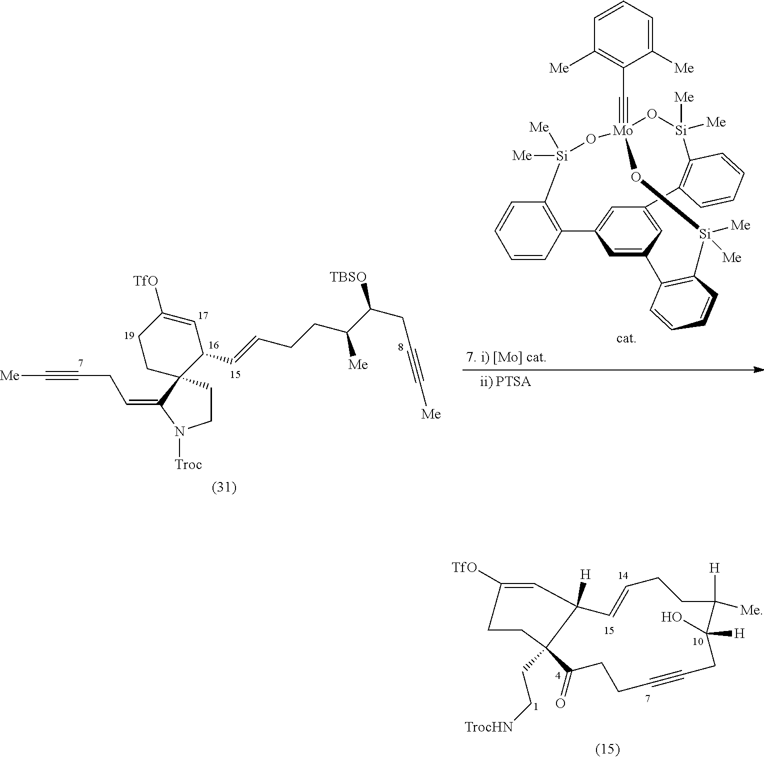

Embodiment 10. The process of Embodiment 9, further comprising ring formation and oxidation of Compound (15) to form Compound (16):

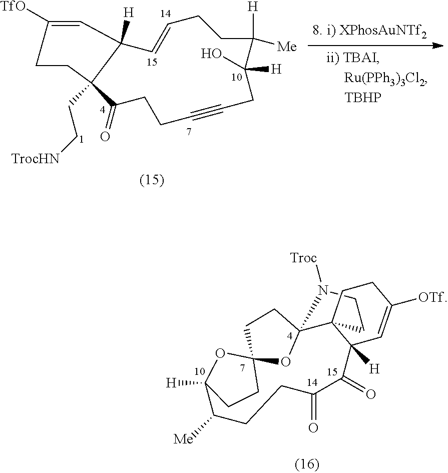

Embodiment 11. The process of Embodiment 10, further comprising the reduction of Compound (16) to Compound (32):

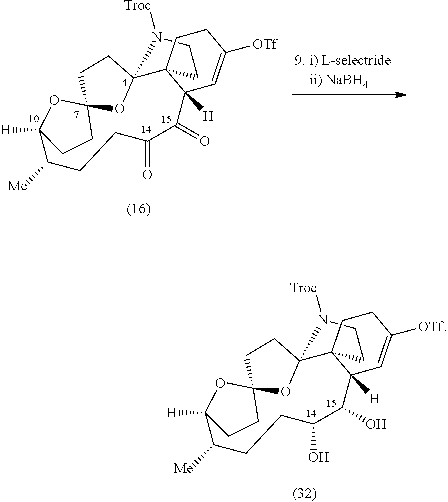

Embodiment 12. The process of Embodiment 11, further comprising ring-chain tautomerism of Compound (32) to form Compound (19):

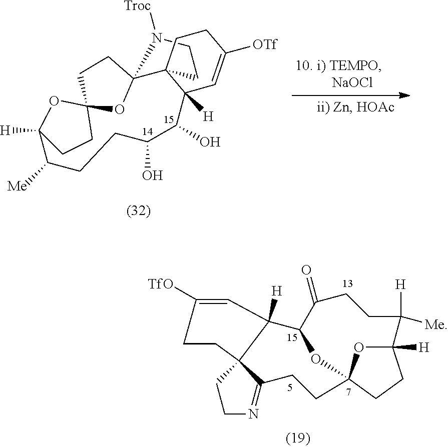

Embodiment 13. The process of Embodiment 12, further comprising the silylation and oxidation of Compound (19) to form Compound (20):

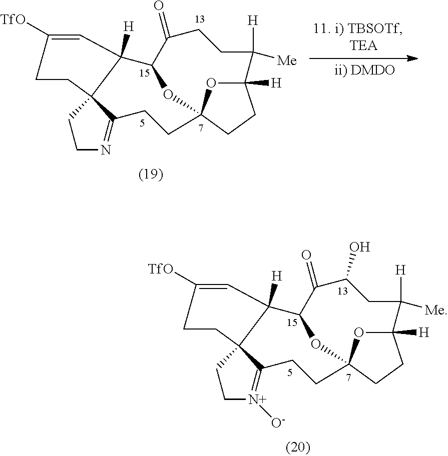

Embodiment 14. The process of Embodiment 13, further comprising the conversion of Compound (20) to Compound (21):

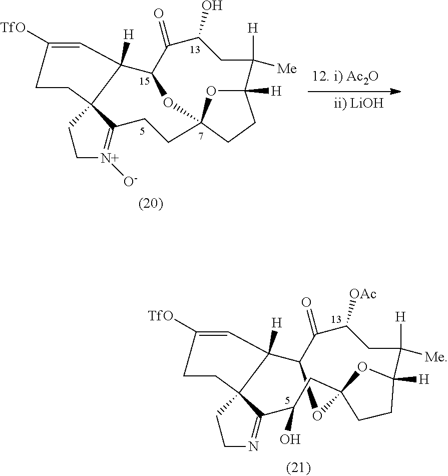

Embodiment 15. The process of Embodiment 14, further comprising Pd-catalyzed vinylation of Compound (21) to form Compound (22):

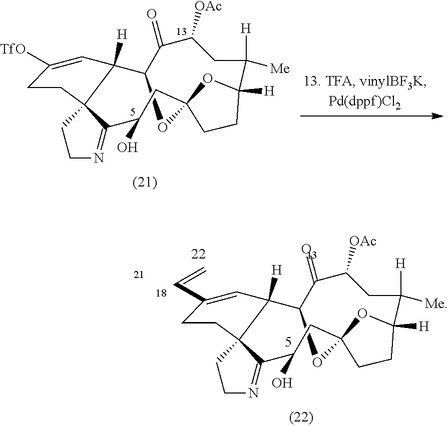

Embodiment 16. The process of Embodiment 15, further comprising the Dess-Martin oxidation of Compound (22) and subsequent hydrolysis to form Compound (2):

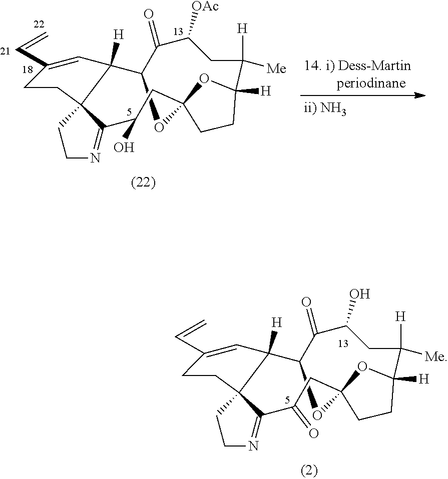

Embodiment 17. The process of Embodiment 16, further comprising the reduction of Compound (2) to form Portimine A (1):

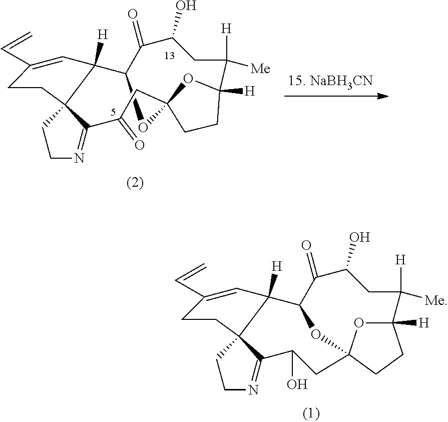

Embodiment 18. A process for preparing Portimine A (1),

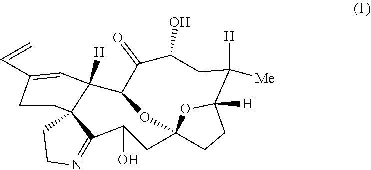

wherein the process comprises the reduction of Compound (2) to form Portimine A (1):

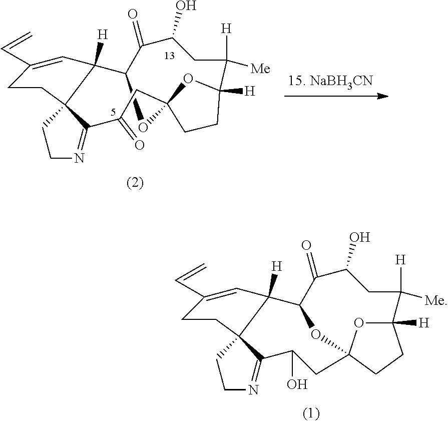

Embodiment 19. A compound having the structure of Formula (2):

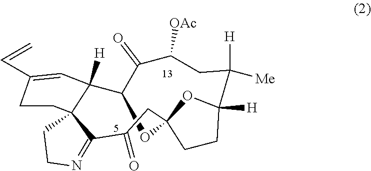

or a scalemic or racemic mixture of.

Embodiment 20. A process of preparing the compound of Embodiment 19, comprising the Dess-Martin oxidation of Compound (22) and subsequent hydrolysis to form Compound (2)

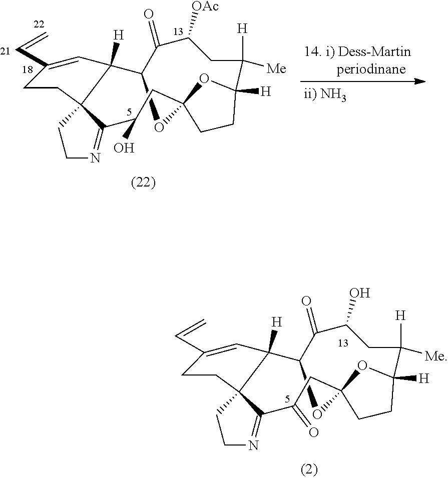

Embodiment 21. A compound having the structure of Formula (22):

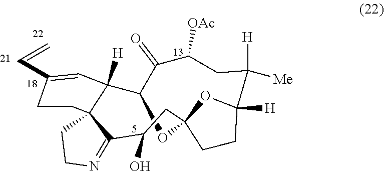

or a scalemic or racemic mixture of.

Embodiment 22. A process of preparing the compound of Embodiment 21, comprising the Pd-catalyzed vinylation of Compound (21) to form Compound (22):

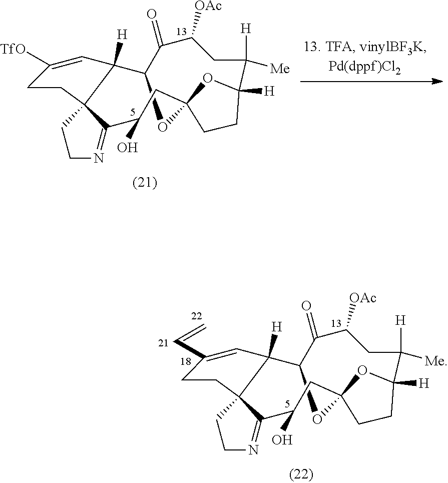

Embodiment 23. A compound having the structure of Formula (21):

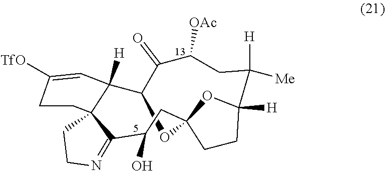

or a scalemic or racemic mixture of.

Embodiment 24. A process of preparing the compound of Embodiment 23, comprising the conversion of Compound (20) to Compound (21):

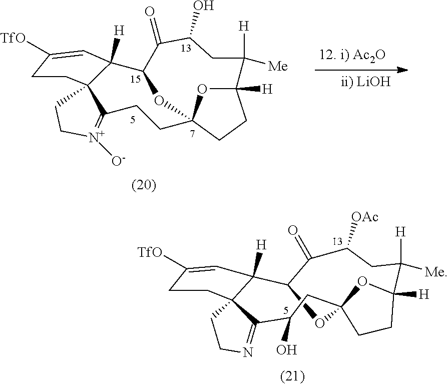

Embodiment 25. A compound having the structure of Formula (20):

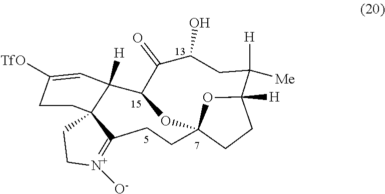

or a scalemic or racemic mixture of.

Embodiment 26. A process of preparing the compound of Embodiment 25, comprising the silylation and oxidation of Compound (19) to form Compound (20):

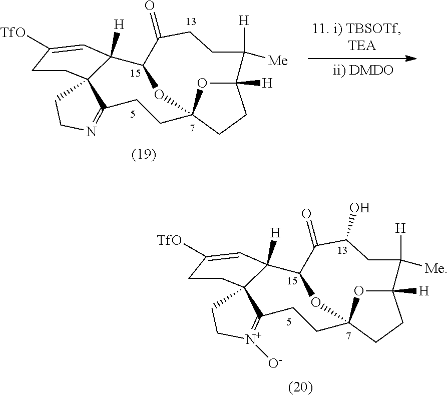

Embodiment 27. A compound having the structure of Formula (19):

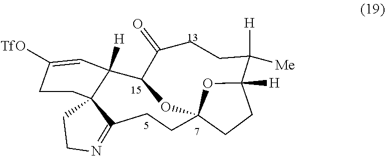

or a scalemic or racemic mixture of.

Embodiment 28. A process of preparing the compound of Embodiment 27, comprising ring-chain tautomerism of Compound (32) to form Compound (19):

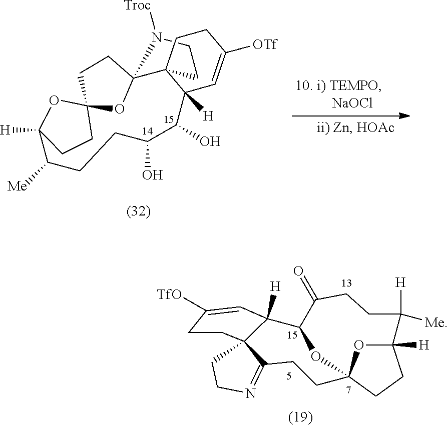

Embodiment 29. A compound having the structure of Formula (32):

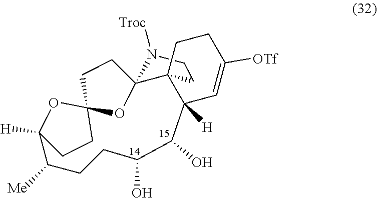

or a scalemic or racemic mixture of.

Embodiment 30. A process of preparing the compound of Embodiment 29, comprising the reduction of Compound (16) to Compound (32):

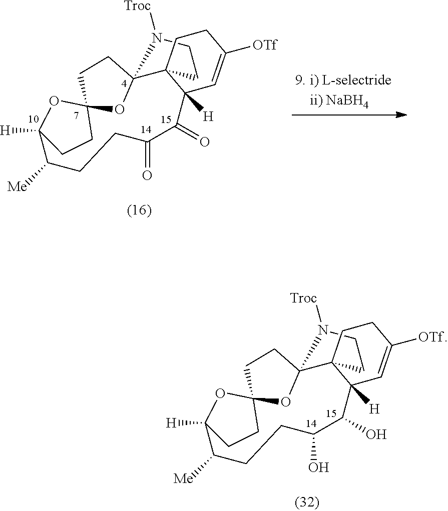

Embodiment 31. A compound having the structure of Formula (16):

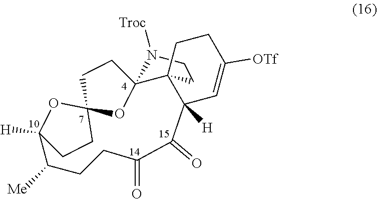

or a scalemic or racemic mixture of.

Embodiment 32. A process of preparing the compound of Embodiment 31, comprising ring formation and oxidation of Compound (15) to form Compound (16):

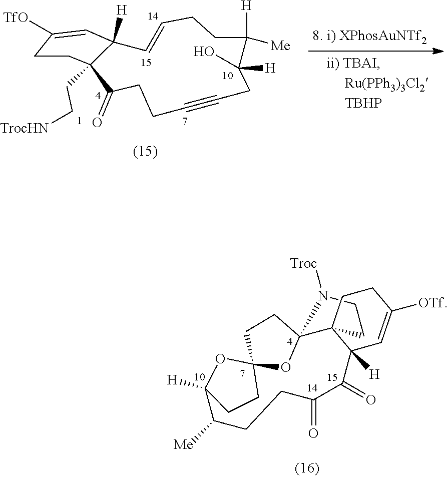

Embodiment 33. A compound having the structure of Formula (15):

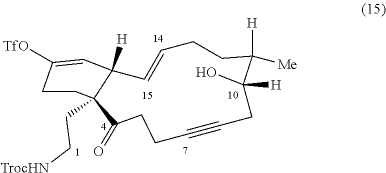

or a scalemic or racemic mixture of.

Embodiment 34. A process of preparing the compound of Embodiment 33, comprising Mo-catalyzed ring-closing alkyne metathesis to form Compound (15):

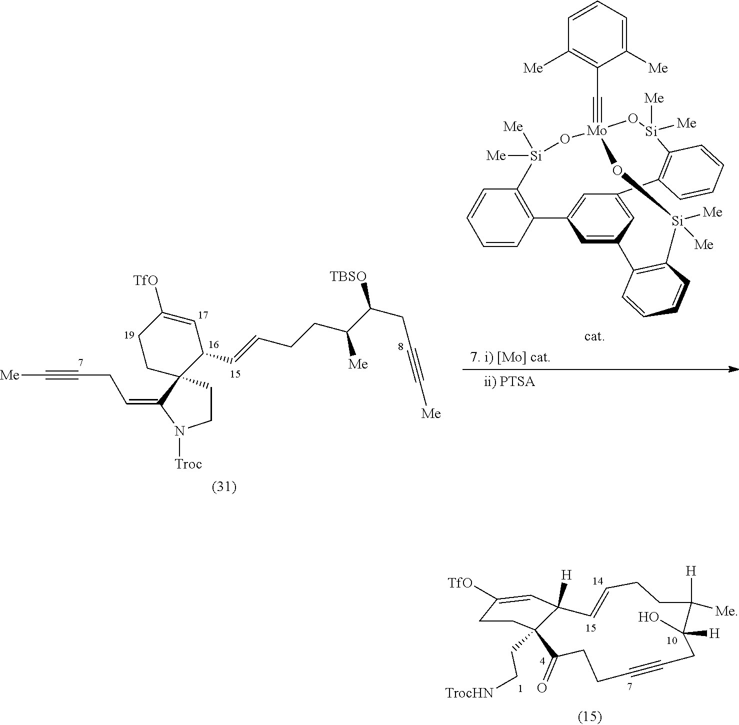

Embodiment 35. A compound having the structure of Formula (31):

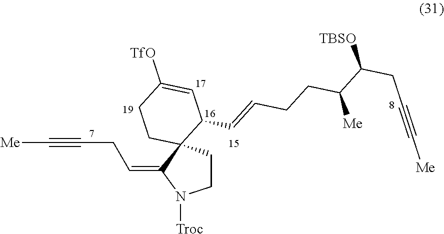

or a scalemic or racemic mixture of.

Embodiment 36. A process of preparing the compound of Embodiment 35, comprising the conversion of compound (7) to the N-protected imine Compound (31):

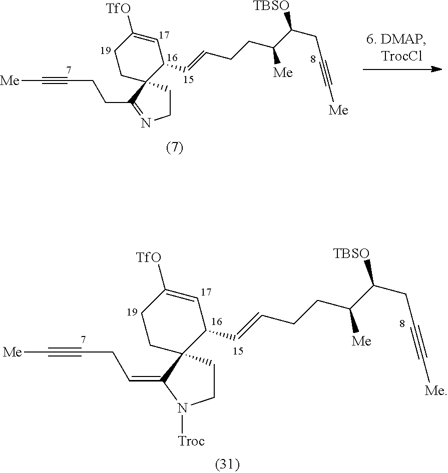

Embodiment 37. A compound havingthe structure of Formula (7):

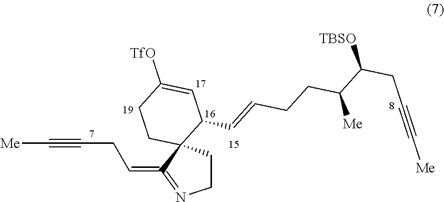

or a scalemic or racemic mixture of.

Embodiment 38. A process of preparing the compound of Embodiment 37, comprising the transmetallation of Compound (13) and conjugate addition with Compound (12) to form Compound (7):

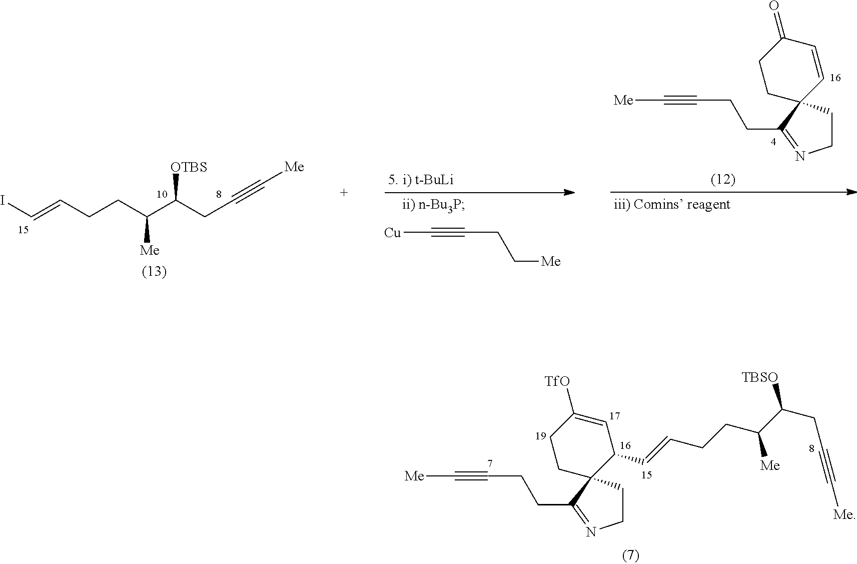

Embodiment 39. A compound having the structure of Formula (28):

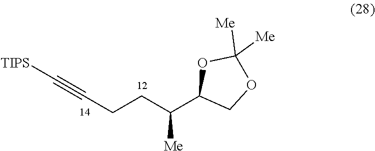

or a scalemic or racemic mixture of.

Embodiment 40. A process of preparing the compound of Embodiment 39 from (S)-solketal, comprising the following steps:

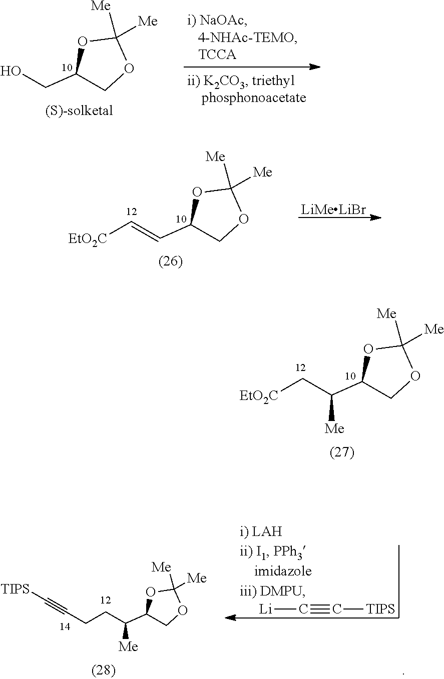

Embodiment 41. A compound having the structure of Formula (29):

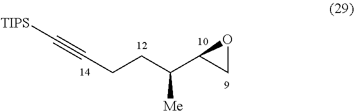

or a scalemic or racemic mixture of.

Embodiment 42. A process of preparing the compound of Embodiment 41 from Compound (28), comprising the following step:

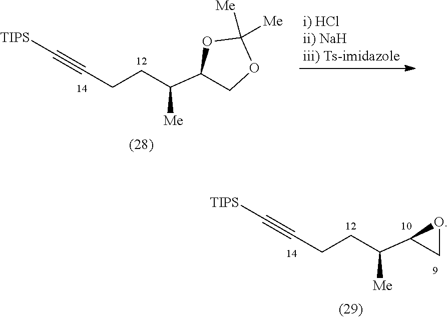

Embodiment 43. A compound having the structure of Formula (30):

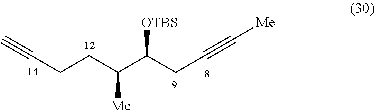 [SMILES: C#CCC[C@H](C)[C@H](CC#CC)O[Si](C)(C)C(C)(C)C]

or a scalemic or racemic mixture of.

Embodiment 44. A process of preparing the compound of Embodiment 43 from Compound (28), comprising the following step:

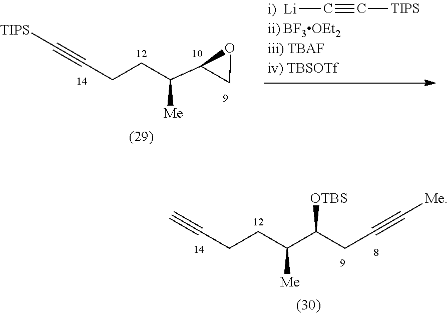

Embodiment 45. A compound having the structure of Formula (13):

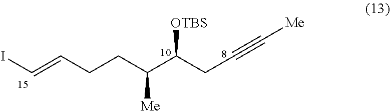 [SMILES: CC#CC[C@H](O[Si](C)(C)C(C)(C)C)[C@@H](C)CC/C=C/I]

or a scalemic or racemic mixture of.

Embodiment 46. A process of preparing the compound of Embodiment 45 from (S)-solketal, comprising the following steps:

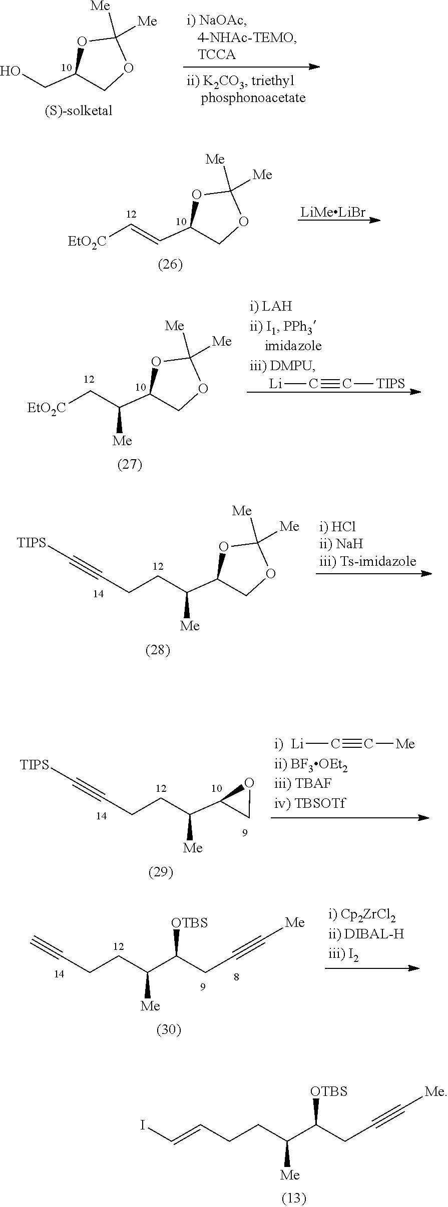

Embodiment 47. A compound having the structure of Formula (12):

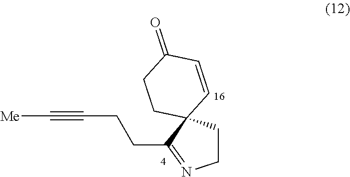 [SMILES: CC#CCCC1=NCC[C@]12C=CC(=O)CC2]

or a scalemic or racemic mixture of.

Embodiment 48. A process of preparing the compound of Embodiment 47, comprising the cyclization of Compound (11) to form Compound (12):

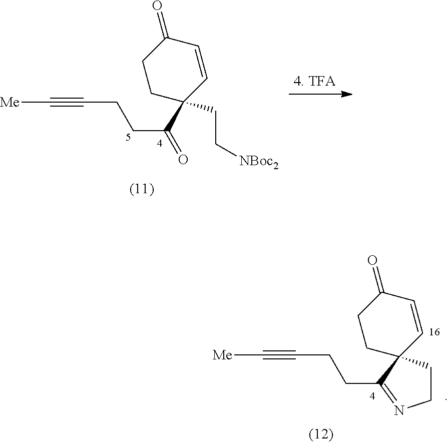

Embodiment 49. A compound having the structure of Formula (11):

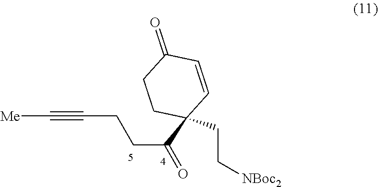

or a scalemic or racemic mixture of.

Embodiment 50. A process of preparing the compound of Embodiment 49, comprising the alcohol oxidation of Compound (10), subsequent Grignard addition, and Dess-Martin oxidation to form Compound (11):

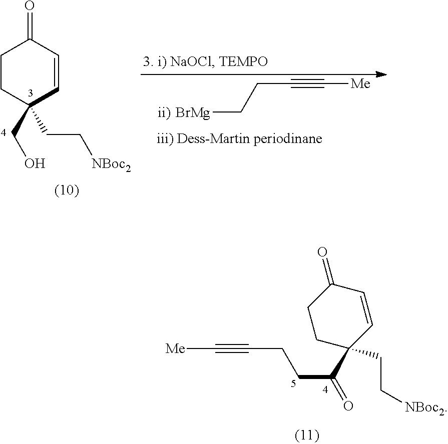

Embodiment 51. A compound having the structure of Formula (10):

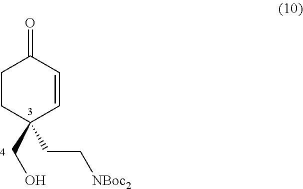

or a scalemic or racemic mixture of.

Embodiment 52. A process of preparing the compound of Embodiment 51, comprising Diels-Alder cycloaddition of Compound (8) to form Compound (10):

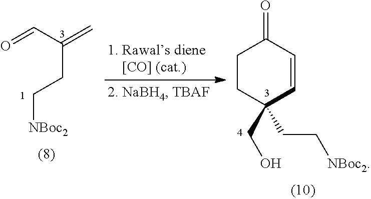

Embodiment 53. A compound having the structure of Formula (8):

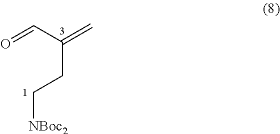

or a scalemic or racemic mixture of.

Embodiment 54. A process of preparing the compound of Embodiment 53 having the Formula of compound (8), comprising conversion of Compound (23) to form Compound (8):

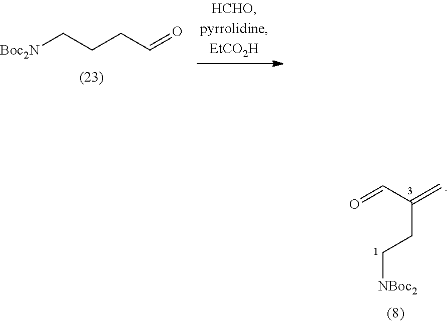

Embodiment 55. A process for preparing Portimine A (1),

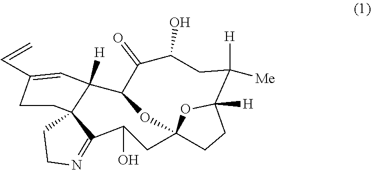

the process comprising the steps of:
i) the reduction of Compound (2) to form Portimine A (1):

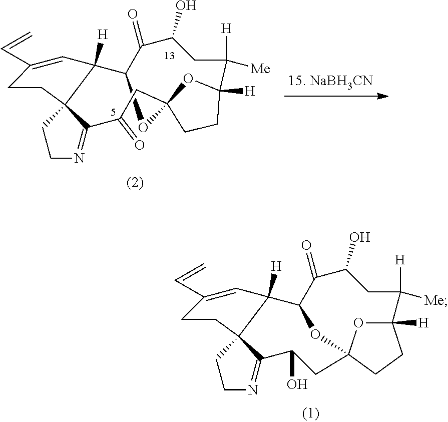

ii) the Dess-Martin oxidation of Compound (22) and subsequent hydrolysis to form Compound (2)

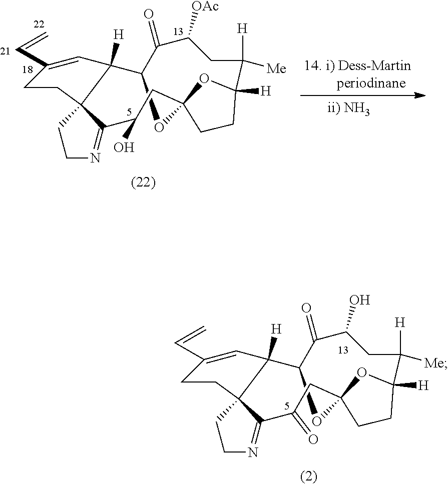

iii) the Pd-catalyzed vinylation of Compound (21) to form Compound (22):

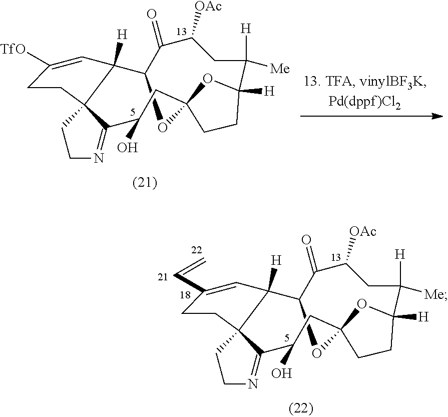

iv) the conversion of Compound (20) to Compound (21):

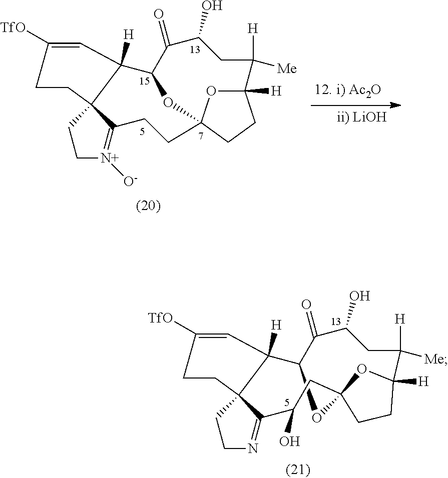

v) the silylation and oxidation of Compound (19) to form Compound (20):

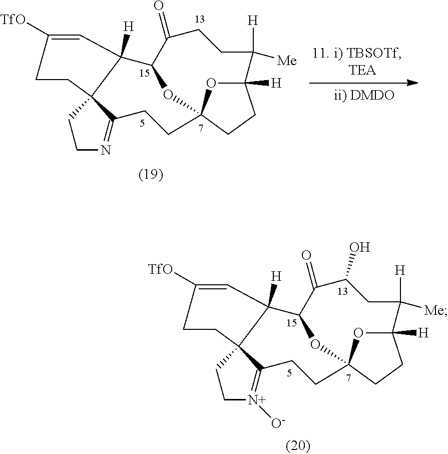

vi) ring-chain tautomerism of Compound (32) to form Compound (19):

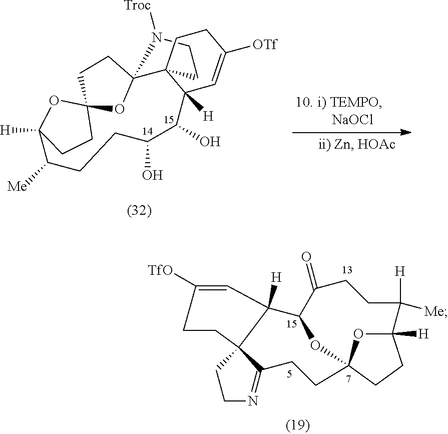

vii) the reduction of Compound (16) to Compound (32):

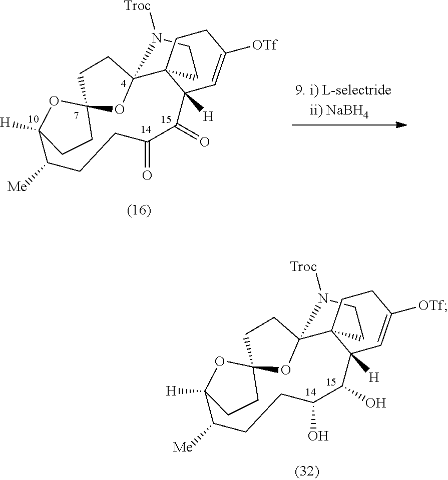

viii) ring formation and oxidation of Compound (15) to form Compound (16):

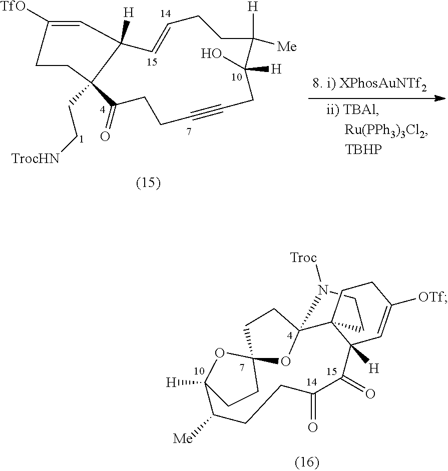

ix) Mo-catalyzed ring-closing alkyne metathesis to form Compound (15):

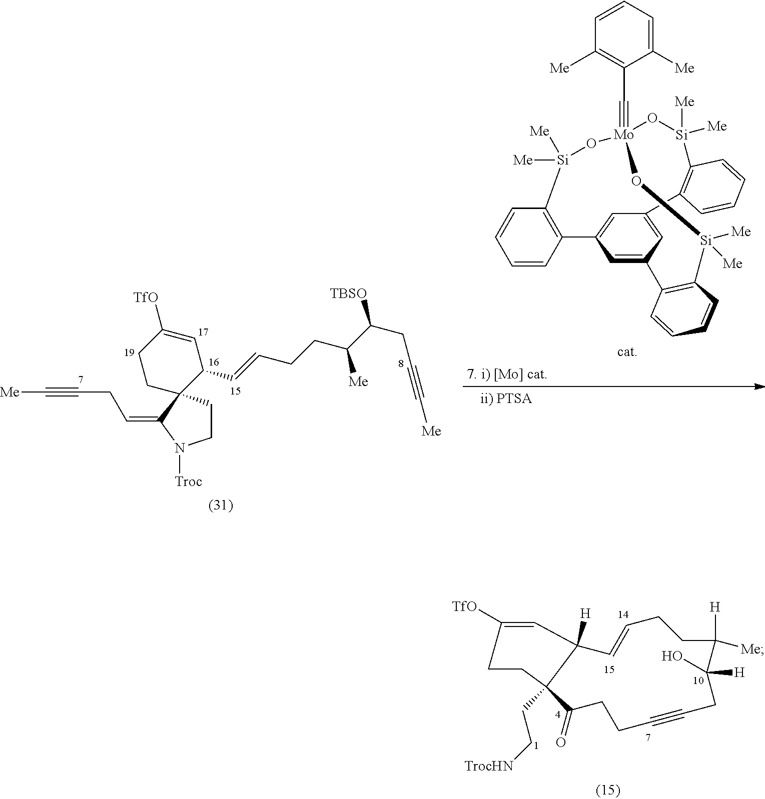

x) the conversion of compound (7) to the N-protected imine Compound (31):

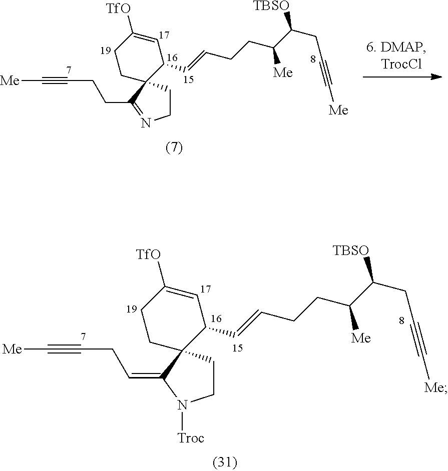

xi) the transmetallation of Compound (13) and conjugate addition with Compound (12) to form Compound (7):

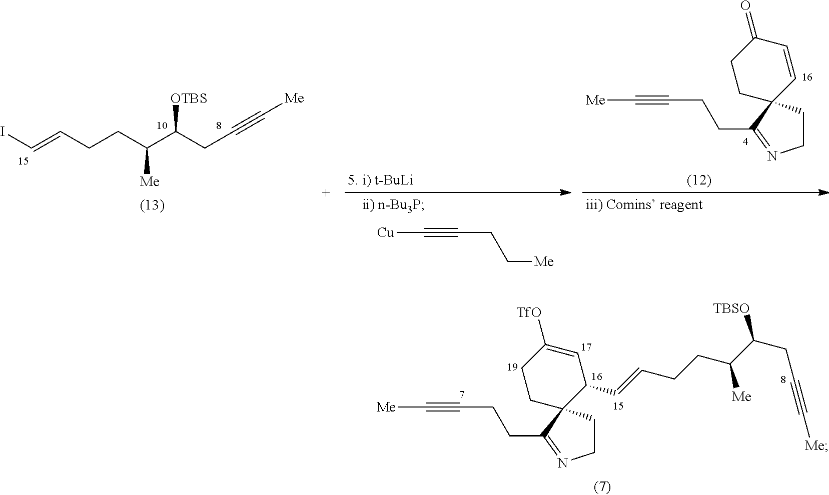

xii) the cyclization of Compound (11) to form Compound (12):

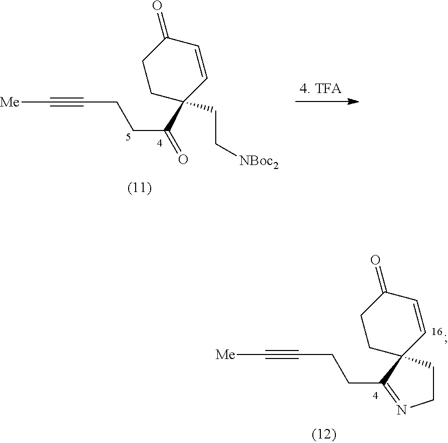

xiii) the alcohol oxidation of Compound (10), subsequent Grignard addition, and Dess-Martin oxidation to form Compound (11):

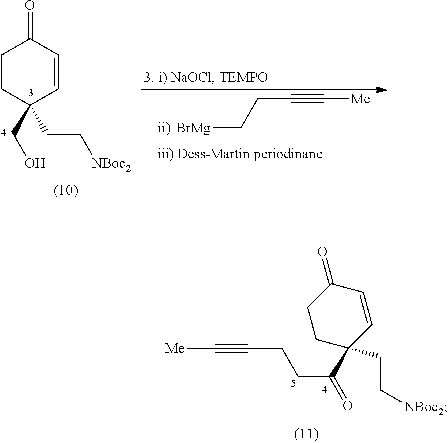

xiv) Diels-Alder cycloaddition of Compound (8) to form Compound (10):

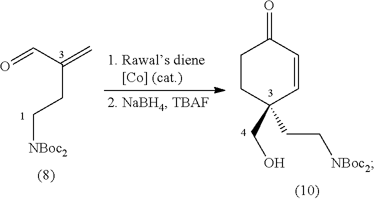

and
xv) conversion of Compound (23) to form Compound (8):

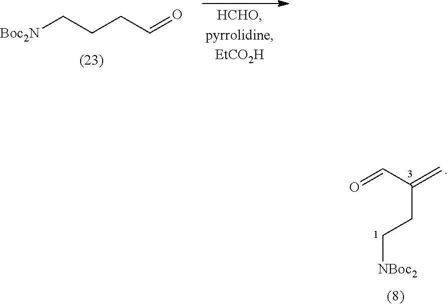

Embodiment 56. A compound having the structure of Formula (33-1):

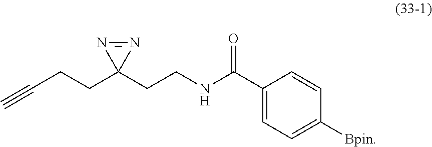

Embodiment 57. A process of preparing the compound of Embodiment 56 from Compound (33-0), comprising the following step:

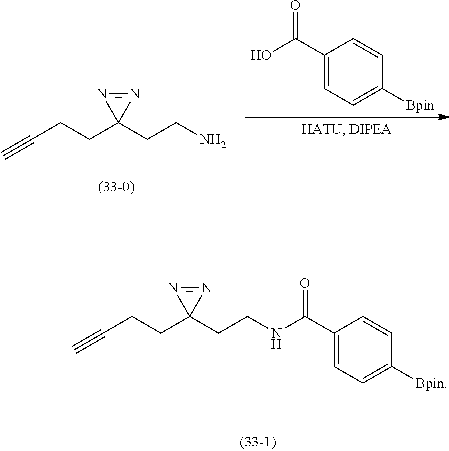

Embodiment 58. A compound having the structure of Formula (33-2):

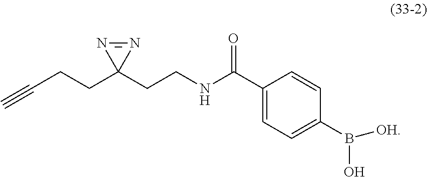 [SMILES: C#CCCC1(CCNC(=O)c2ccc(B(O)O)cc2)N=N1]

Embodiment 59. A process of preparing the compound of Embodiment 58 from Compound (33-0), comprising the following step:

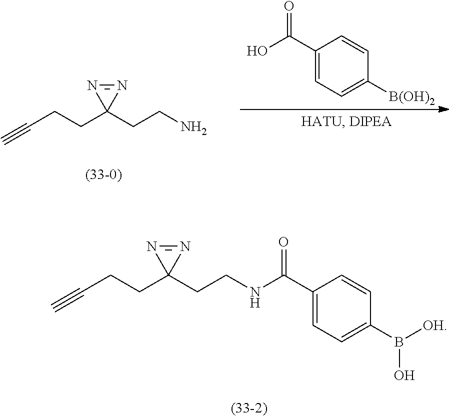

Embodiment 60. A compound having the structure of Formula (34):

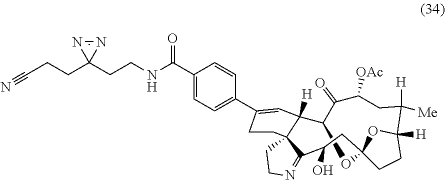

or a scalemic or racemic mixture of.

Embodiment 61. A process of preparing the compound of Embodiment 60 from Compound (21), comprising the following step:

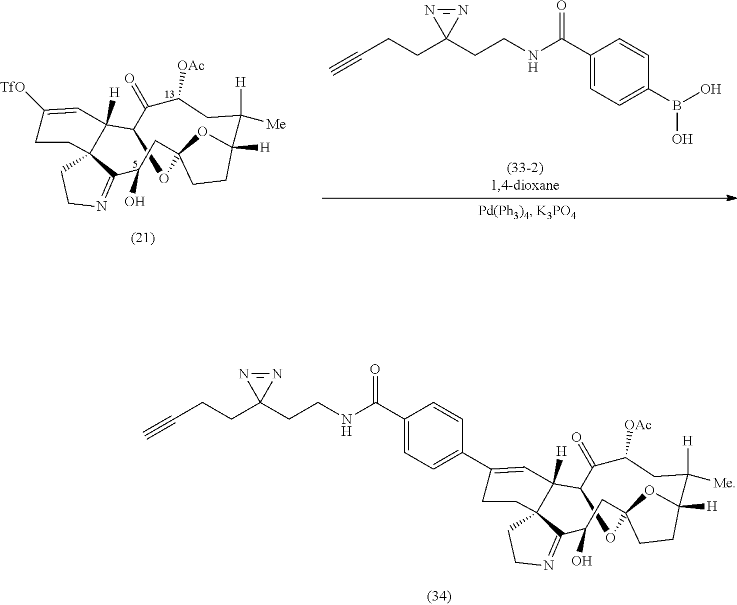

Embodiment 62. A compound having the structure of Formula (35-1):

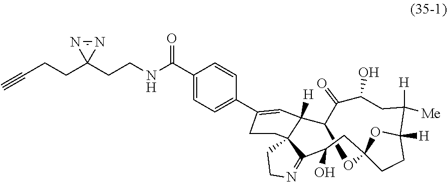

or a scalemic or racemic mixture of.

Embodiment 63. A process of preparing the compound of Embodiment 62 from Compound (21), comprising the following step:

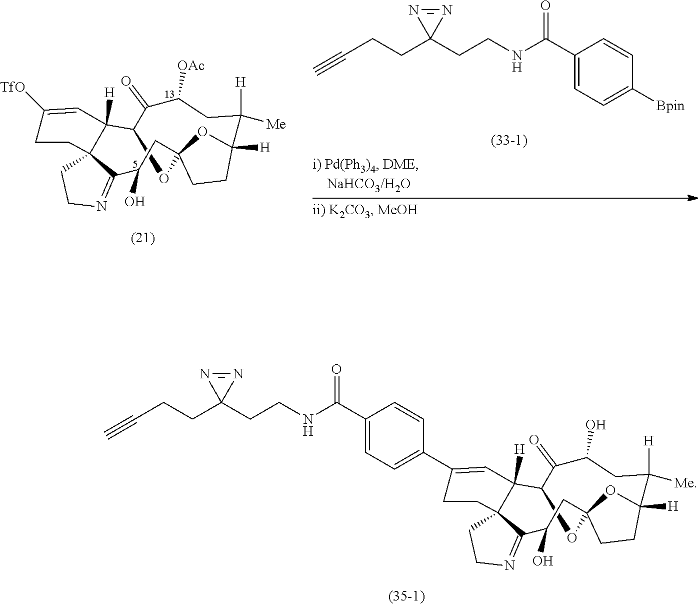

Embodiment 64. A compound having the structure of Formula (35-2):

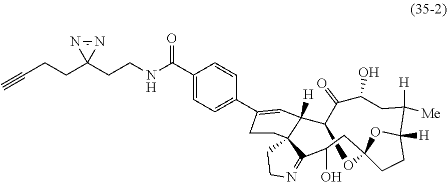

or a scalemic or racemic mixture of.

Embodiment 65. A process of preparing the compound of Embodiment 64 from Compound (35-2), comprising the following step:

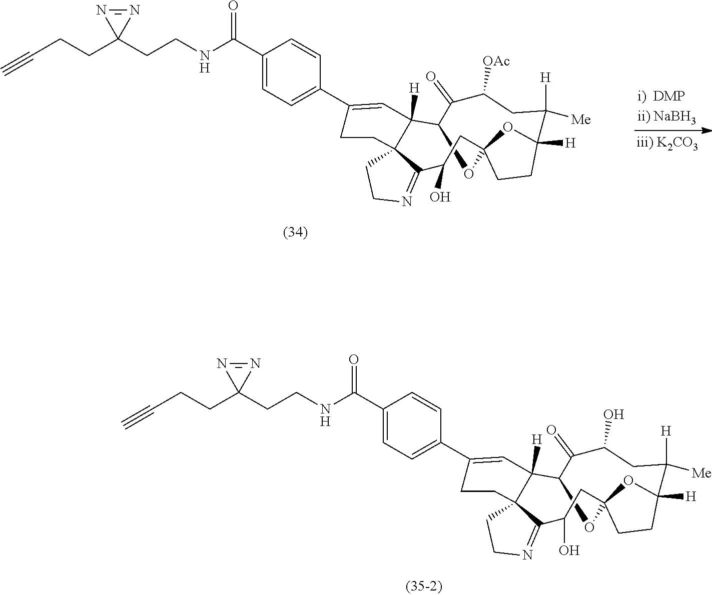

Embodiment 66. A compound having the structure of Formula (36):

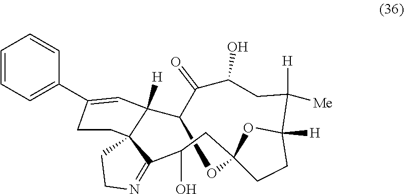

or a scalemic or racemic mixture of.

Embodiment 67. A process of preparing the compound of Embodiment 66 from Compound (39), comprising the following step:

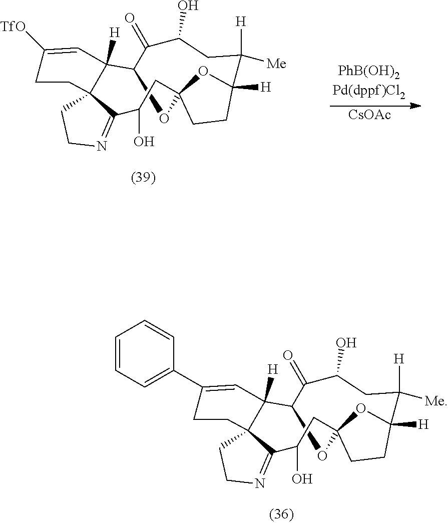

Embodiment 68. A compound having the structure of Formula (37):

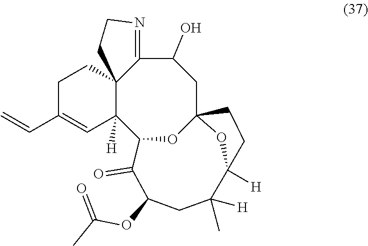 [SMILES: C=CC1=C[C@@H]2[C@@H]3O[C@]4(CC[C@H](O4)C(C)C[C@@H](OC(C)=O)C3=O)CC(O)C3=NCC[C@@]32CC1]

or a scalemic or racemic mixture of.

Embodiment 69. A process of preparing the compound of Embodiment 68 from Compound (22), comprising the following step:

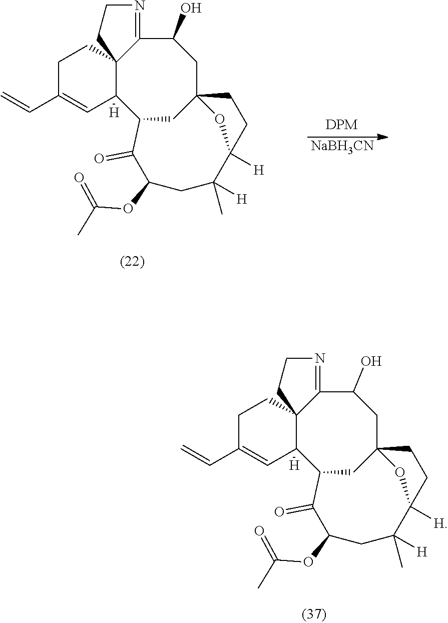

Embodiment 70. A compound having the structure of Formula (38):

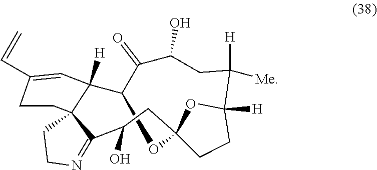

Embodiment 71. A process of preparing the compound of Embodiment 70 from Compound (21), comprising the following step:

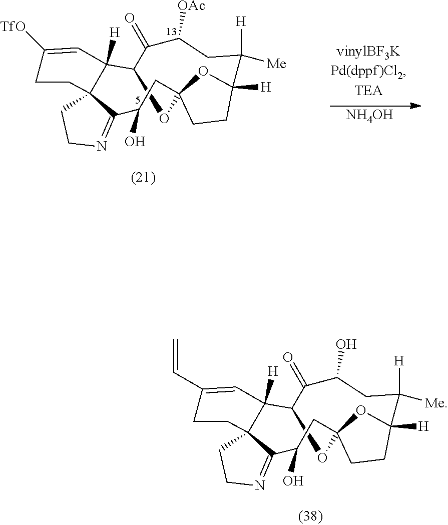

Embodiment 72. A compound having the structure of Formula (39):

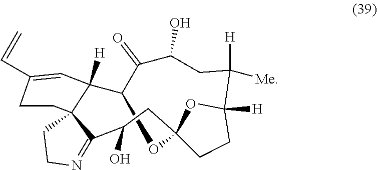

Embodiment 73. A process of preparing the compound of Embodiment 72 from Compound (21), comprising the following step:

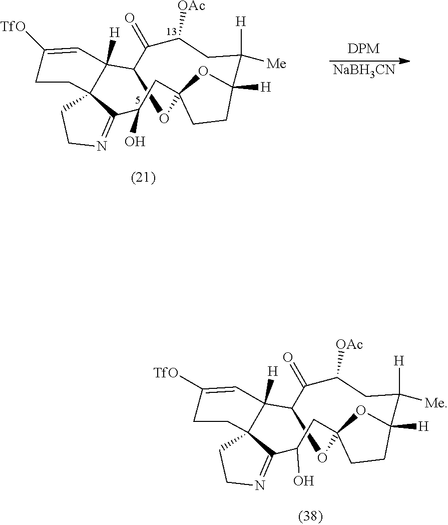

Embodiment 74. A compound having the structure of Formula (40):

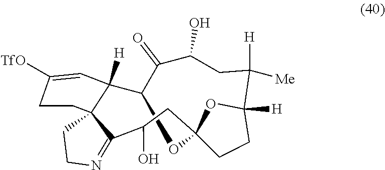

or a scalemic or racemic mixture of.

Embodiment 75. A process of preparing the compound of Embodiment 74 from Compound (39), comprising the following step:

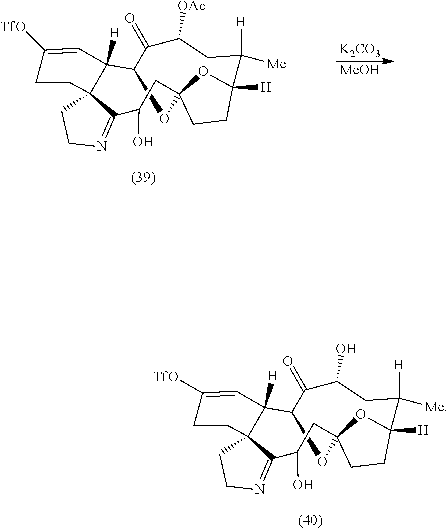

Embodiment 76. A process of preparing diverse analogues of Portimine A, comprising the following steps:

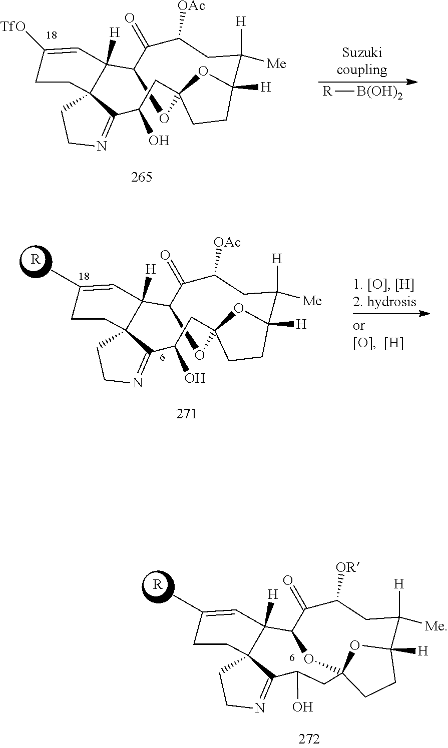

wherein:
R is (C₆-C₁₀) aryl, (C₂-C₆) alkenyl, or (C₅—Cl₂) heteroaryl, wherein each aryl is optionally substituted with —NH(CH₂)₂C(—N═N—)(CH₂)₂(C≡CH); and
R′ is H or Ac;
including enantiomers, scalemic and racemic mixtures, and pharmaceutically acceptable salts thereof.

Embodiment 77. A compound prepared by the process of Claim 76, wherein the compound is selected from the group consisting of:

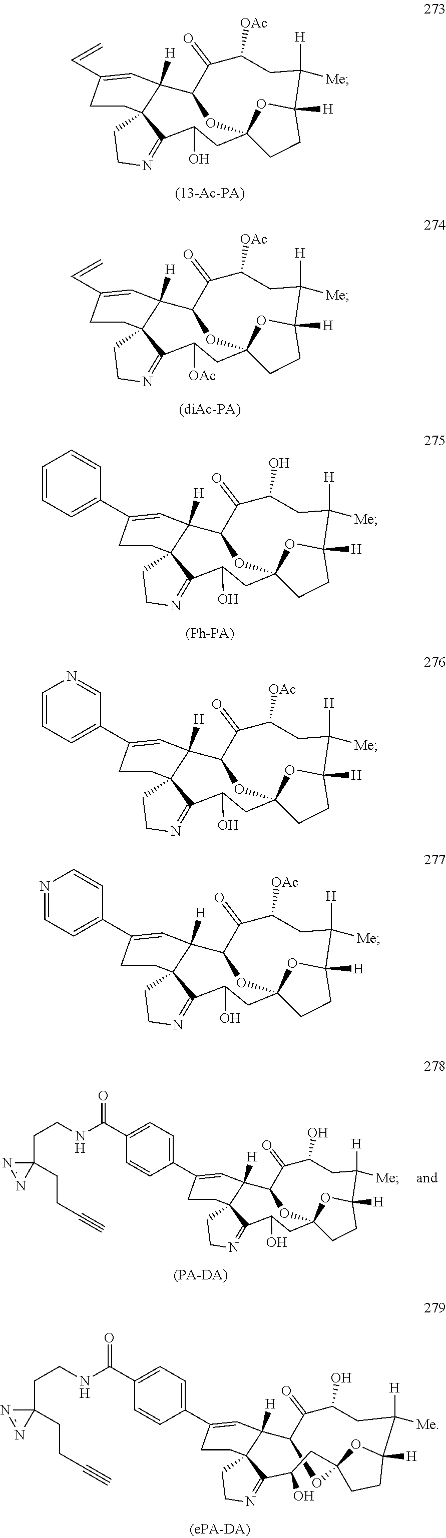

Embodiment 78. A process of preparing diverse analogues of Portimine A, comprising the following steps:

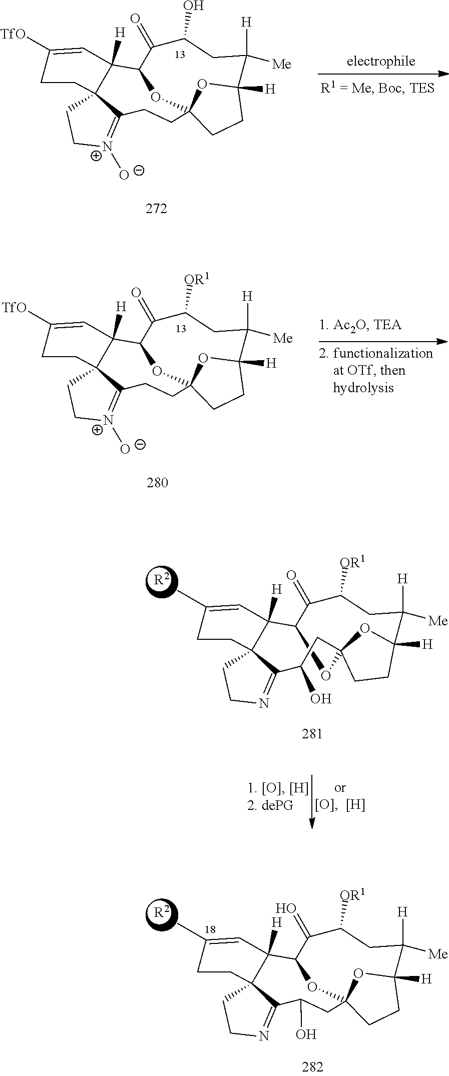

wherein:
R¹ is H, Me, Boc, or TES; and
R² is H, halo, (C₆-C₁₀) aryl, (C₂-C₆) alkenyl, or (C₅—Cl₂) heteroaryl, wherein each aryl is optionally substituted with —NH(CH₂)₂C(—N═N—)(CH₂)₂(C≡CH).
R′ is H or Ac;
including enantiomers, scalemic and racemic mixtures, and pharmaceutically acceptable salts thereof.

Embodiment 79. A compound prepared by the process of Claim 78, wherein the compound is selected from the group consisting of:

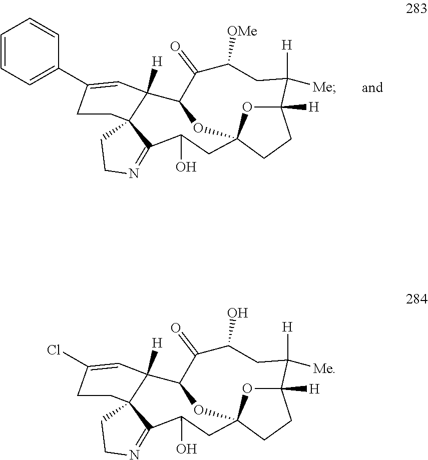

Embodiment 80. A process of preparing diverse analogues of Portimine A, comprising the following steps:

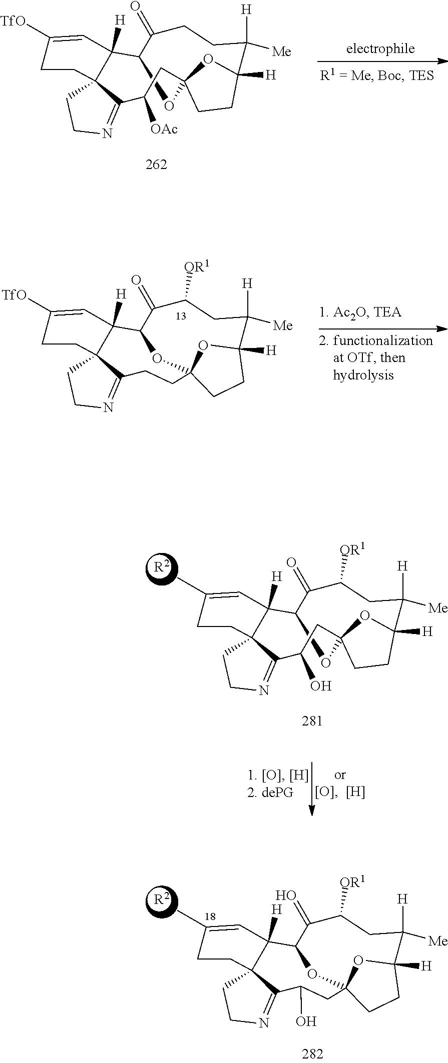

wherein:
R¹ is H, Me, Boc, or TES; and
R² is H, halo, (C₆-C₁₀) aryl, (C₂-C₆) alkenyl, or (C₅—Cl₂) heteroaryl, wherein each aryl is optionally substituted with —NH(CH₂)₂C(—N═N—)(CH₂)₂(C≡CH).
R′ is H or Ac;
including enantiomers, scalemic and racemic mixtures, and pharmaceutically acceptable salts thereof.

Embodiment 81. A compound prepared by the process of Claim 80, wherein the compound is selected from the group consisting of:

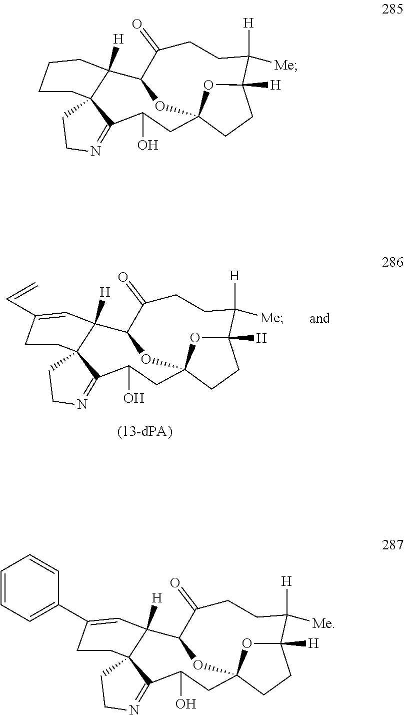

Embodiment 82. The process of any one of Claims 76, 78, or 80, wherein the Portimine A analogue product is useful in forming an antibody-drug conjugate (ADC) for the treatment of cancer.

Embodiment 83. A method of forming antibody-drug conjugate (ADC) for the treatment of cancer, comprising use of the compound of any one of Claims 76-81.

Embodiment 84. A method of treating cancer, comprising administering to a patient in need thereof a pharmaceutically effective amount of any one of Claims 76-81 capable of forming antibody-drug conjugate (ADC).

Embodiment 85. Any process, compound, method, or mixture as described herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “~~~~” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)=CH—), amide/imidic acid (—C(═O)—NH—⇄—C(—OH)=N—) and amidine (—C(═NR)—NH—⇄—C(—NHR)=N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₅), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to a cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino,dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5—membered heteroaryl groups containing 1 heteroatom include, without limitation, pyrrolyl, furanyl and thiophenyl. Exemplary 5—membered heteroaryl groups containing 2 heteroatoms include, without limitation, imidazolyl, pyrazolyl, oxazolyl, isoxazolyl, thiazolyl, and isothiazolyl. Exemplary 5—membered heteroaryl groups containing 3 heteroatoms include, without limitation, triazolyl, oxadiazolyl, and thiadiazolyl. Exemplary 5—membered heteroaryl groups containing 4 heteroatoms include, without limitation, tetrazolyl. Exemplary 6-membered heteroaryl groups containing 1 heteroatom include, without limitation, pyridinyl. Exemplary 6—membered heteroaryl groups containing 2 heteroatoms include, without limitation, pyridazinyl, pyrimidinyl, and pyrazinyl. Exemplary 6—membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR′°)R^{bb}, —SH, —SR—, —C(═O)R^{aa}, —CO₂H, —CHO, —CO₂R′, —OC(═O)R^{aa}, —OCO₂R^{aa}, —C(═O)N(R^{bb})₂, —OC(═O)N(R^{bb})₂, —NR^{bb}C(═O)R^{aa} NR^{bb}CO₂R^{aa}, —NR^{bb}C(═O)N(R^{bb})₂, —C(═NR^{bb})R″, —C(═NR^{bb})OR^{aa}, —OC(═NR^{bb})R^{aa}, —OC(═NR^{bb})OR^{aa}, —C(=NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R—, —S(═O)R^{aa}, —OS(═O)R^{aa}, —B(OR^{aa})₂, C₁₋₁₀ alkyl, C₂₋₁₀ alkenyl, C₂₋₁₀ alkynyl, C₃₋₁₄ carbocyclyl, 3- to 14—membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R″ is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14—membered heterocyclyl, C₆₋₁₄ aryl, and 5- to 14—membered heteroaryl, or two R′ groups are joined to form a 3- to 14—membered heterocyclyl or 5- to 14—membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR″, —N(R″)₂, —CN, —C(═O)R″, —C(═O)N(R″)₂, —CO₂R″, —SO₂R″, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14—membered heterocyclyl, C₆₋₁₄ aryl, and 5- to 14—membered heteroaryl, or two Rb groups are joined to form a 3- to 14—membered heterocyclyl or 5- to 14—membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14—membered heterocyclyl, C₆₋₁₄ aryl, and 5- to 14—membered heteroaryl, or two R^{cc} groups are joined to form a 3- to 14—membered heterocyclyl or 5- to 14—membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO2(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl),—OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C_{1 6} alkyl), —SO₂N(C_{1 6} alkyl)₂, —SO₂NH(C_{1 6} alkyl), —SO₂NH₂,—SO₂C_{1 6} alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

As used herein, the term “adjuvant” refers to a compound or composition that enhances a medical treatment, such as an added pharmacological agent added to a drug to increase or aid its effect, such as an immunological agent that increases an antigenic response or otherwise contributes to or enhances an existing medical regimen (for example, Freund's adjuvant).

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term an “amino terminus modification group” refers to any molecule that can be attached to a terminal amine group. By way of example, such terminal amine groups may be at the end of polymeric molecules, wherein such polymeric molecules include, but are not limited to, polypeptides, polynucleotides, and polysaccharides. Terminus modification groups include but are not limited to, various water soluble polymers, peptides or proteins. By way of example only, terminus modification groups include polyethylene glycol or serum albumin. Terminus modification groups may be used to modify therapeutic characteristics of the polymeric molecule, including but not limited to increasing the serum half-life of peptides.

As used herein, the term “antibody fragment” is meant any form of an antibody other than the full-length form. Antibody fragments herein include antibodies that are smaller components that exist within full-length antibodies, and antibodies that have been engineered. Antibody fragments include but are not limited to Fv, Fe, Fab, and (Fab′)₂, single chain Fv (scFv), diabodies, triabodies, tetrabodies, bifunctional hybrid antibodies, CDR1, CDR2, CDR3, combinations of CDR's, variable regions, framework regions, constant regions, heavy chains, light chains, and variable regions, and alternative scaffold non-antibody molecules, bispecific antibodies, and the like (Maynard & Georgiou, 2000, Annu. Rev. Biomed. Eng. 2:339-76; Hudson, 1998, Curr. Opin. Biotechnol. 9:395-402). Another functional substructure is a single chain Fv (scFv), comprised of the variable regions of the immunoglobulin heavy and light chain, covalently connected by a peptide linker (S-z Hu et al., 1996, Cancer Research, 56, 3055-3061). These small (Mr 25,000) proteins generally retain specificity and affinity for antigen in a single polypeptide and can provide a convenient building block for larger, antigen-specific molecules. Unless specifically noted otherwise, statements and claims that use the term “antibody” or “antibodies” specifically includes “antibody fragment” and “antibody fragments.”

As used herein, the term “antibody-drug conjugate, or “ADC”, as used herein, refers to an antibody molecule, or fragment thereof, that is covalently bonded to one or more biologically active molecule(s). The biologically active molecule may be conjugated to the antibody through a linker, polymer, or other covalent bond.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O ³¹P, ³²P ³⁵S, ¹⁸F, ³⁶Cl and ¹²³I Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

### Examples

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC20), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et20), 0-(7-azabenzotriazole-1-yl)-N, N,N‘N’-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO2—(mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe2Si (TBDMS), triethylamine (TEA or Et3N), 2,2,6,6-tetramethylpiperidine 1-oxyl (TEMPO), triflate or CF3SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me3Si (TMS), p-toluenesulfonic acid monohydrate (TsOH or pTsOH), 4-Me-C₆H₄SO₂— or tosyl (Ts), N-urethane-N-carboxyanhydride (UNCA),. Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Compound 23

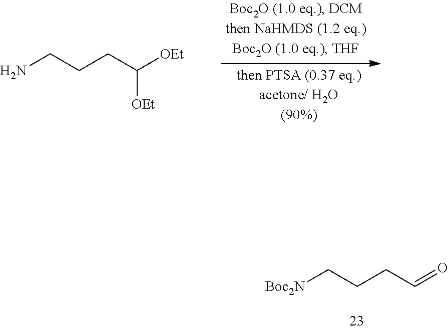

To a solution of 4-aminobutyraldehyde diethyl acetal (32.2 g, 200 mmol) in CH₂Cl₂ (200 mL), was added Boc20 (43.8 g, 201 mmol) dropwise at room temperature. The reaction was monitored by TLC (stain: ninhydrin in ethanol). Upon completion, the solvent was removed under reduced pressure, and the residue was dissolved in THF (200 mL) at 0° C. under argon. To this solution, NaHMDS (0.6 M in toluene, 400 ml, 240 mmol) was added dropwise with vigorous stirring. The resulting solution was kept stirring at the same temperature, followed by a slow addition of Boc20 (43.8 g, 201 mmol). After completion, sat. NH₄Cl aq. was added and stirred overnight. The organic layer was separated, while the aqueous layer was extracted with Et20. The combined organic layer was dried over MgSO₄, followed by concentration to give the crude Boc-carbamate as product.

The crude carbamate obtained above was dissolved in acetone/H₂O (4:1, 500 ml). PTSA monohydrate (14.0 g, 73.6 mmol) was added and stirred at room temperature for 16 h. The mixture was neutralized with saturated aqueous NaHCO₃ and extracted with Et20. The combined organic layer was dried over MgSO₄, filtered and concentrated. The crude product was purified by chromatography (5% EtOAc in hexanes to 15% EtOAc in hexanes) to afford compound 23 (51.7 g, 90%) as colorless oil.

### Physical State: Colorless Oil

TLC: R_{f}=0.41 (²⁵% ethyl acetate in hexanes, stains brown upon KMnO₄ staining)
¹H-NMR (500 MHz, CDCl₃) δ 9.74 (t, J=2.2 Hz, 1H), 3.58 (t, J=7.2 Hz, 2H), 2.43 (t, J=7.2 Hz, 2H), 1.86 (quin, J=7.2 Hz, 2H), 1.47 (s, 18H)
¹³C-NMR (125 MHz, CDCl₃) δ 201.6, 152.7, 82.6, 45.5, 41.1, 28.1, 21.6

All spectral data meet with reported.¹

Large scale synthesis of Rawal's diene

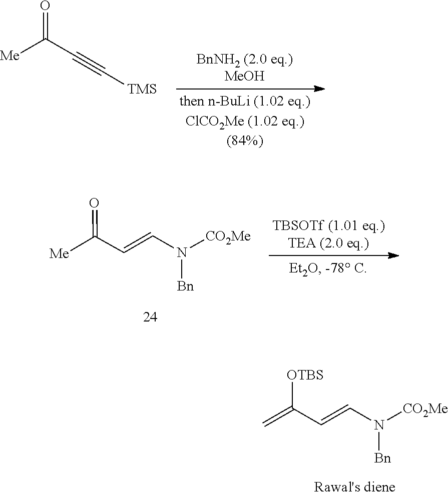 [SMILES: CC(=O)C#C[Si](C)(C)C ; COC(=O)N(/C=C/C(C)=O)Cc1ccccc1 ; C=C(/C=C/N(Cc1ccccc1)C(=O)OC)O[Si](C)(C)C(C)(C)C]

To a solution of BnNH₂ (30.6 g, 286 mmol) in methanol (200 mL) at 0° C. was added 4-(trimetylsilyl)-3-butyn-2-one (21.6 mL, 143 mmol) dropwise. After stirring for 1.0 h at room temperature, the solvent was removed in vacuo. The crude product was suspended in EtOAc and rinsed by aq. NaH₂PO₄ to remove benzylamine. The organic layer was separated and the aqueous phase was extracted with EtOAc for 3 times. The combined organic layer was dried over MgSO₄, and concentrated under reduced pressure to give the crude enamine², which was directly used for next step.

The crude enamine obtained above was dissolved in THE (10 ml/g), and and n-BuLi (2.5 M, 60 ml, 150 mmol) was added at 0° C. under argon over 30 min. After 10 mins, ClCO₂Me (11.6 ml, 14.2 g, 150 mmol) was added slowly, and the solution was stirred for 2 h. Upon completion, the mixture was poured into H₂O (500 ml). The mixture was extracted with EtOAc for 3 times, and dried over MgSO₄. Compound 24 (27.9 g, 120 mmol, 84%) can be obtained by chromatography (0-40% EtOAc in hexanes).

Physical state: pale yellow oil

TLC: R_{f}=0.28 (30% EtOAc in hexanes, UV active, stains brown upon KMnO₄ staining)

¹H-NMR (400 MHz, CDCl₃) δ 8.17 (d, J=14.4 Hz, 1H), 7.36-7.03 (m, 5H), 5.50 (d, J=14.4 Hz, 1H), 4.76 (s, 2H), 3.84 (s, 3H), 2.13 (s, 3H)

¹³C-NMR (100 MHz, CDCl₃) δ 197.2, 154.5, 141.9, 135.3, 128.8, 127.6, 126.4, 109.9, 54.4, 48.2

HRMS (ESI-TOF): calc'd for C₁₃H₁₅NO₃ [M+H]⁺: 234.1125, found: 234.1126 The pure compound 24 (52 g, 223 mmol) was dissolved in Et20 (650 ml) under argon. To this solution, TEA (45.5 g, 450 mmol) was added in one portion at −78° C. TBSOTf (59.5 g, 225 mmol) was dropped into this solution slowly. A cloudy mixture was generated during addition. After stirring at −78° C. for 30 min, the flask was gradually warmed to 0° C. for another 30 min. The reaction mixture was quenched by a mixture of hexanes and sat. NaHCO₃ aq., followed by extraction with hexanes. The organic phase was dried over MgSO₄, and concentrated to give Rawal's diene (purity indicated by NMR, used directly for next step).

Physical state: pale yellow solid

TLC: R_{f}=0.43 (hexanes+1% TEA, UV active, stains brown upon KMnO₄ staining)

¹H-NMR (400 MHz, CDCl₃) δ 7.72-7.42 (m, 1H), 7.32 (t, J=7.3 Hz, 2H), 7.27-7.13 (m, 3H), 5.36 (d, J=14.1 Hz, 2H), 4.80 (s, 2H), 4.13 (s, 1H), 4.07 (s, 1H), 3.83 (s, 3H), 1.00 (s, 9H), 0.18 (s, 6H)

¹³C-NMR (100 MHz, CDCl₃) δ 155.1, 154.5, 136.9, 128.8, 127.3, 126.7, 126.3, 108.4, 93.0, 53.8, 48.2, 26.0, 18.4, −4.5

HRMS (ESI-TOF): calc'd for C₁₉H₂₉NO₃Si [M+H]⁺: 348.1990, found: 348.1986 Compound 25

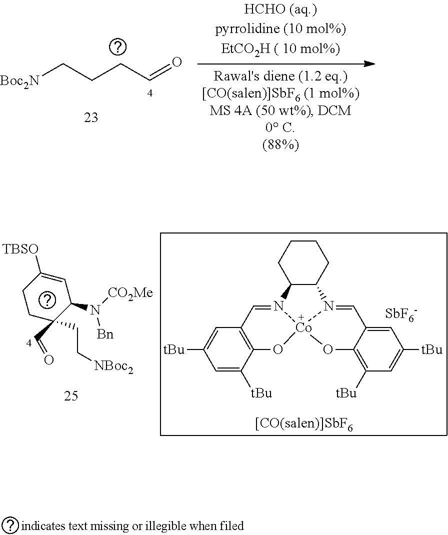

To a mixture of aqueous formaldehyde solution (13 ml, 37% formaldehyde in water) and compound 23 (46.8 g, 162.9 mmol) in i-PrOH (95 mL) were added propionic acid (1.20 g, 16.2 mmol, 0.1 eq.) and pyrrolidine (1.15 g, 16.2 mmol, 0.1 equiv.). The reaction mixture was stirred at 55° C. for 1.0 h. Upon completion, sat. NaHCO₃ aq. and brine was added, and the mixture was extracted with Et20. The combined organic layer was dried over MgSO₄, and concentrated in vacuo to remove all volatiles. The crude acrolein (compound 8) obtained was directly used for Diels-Alder cycloaddition.

Compound 8 was dissolved in DCM (200 mL), followed by adding MS4 Å (25 g, dried in oven) and Rawal's diene (70.5 g, 10.7 mmol, 1.2 equiv.) under argon. After stirring the mixture at 0° C. for 15 mins, [Co(salen)]SbF₆ (984 mg, 1.63 mmol, 0.01 equiv.) was added in one portion, the mixture was stirred under ice bath overnight. After completion, the reaction was diluted with DCM and added silica gel. After concentration in vacuo, the crude was purified with chromatography (dry loading, 0-20% EtOAc in hexanes) to afford compound 25 (92.7 g, 143.3 mmol, 88%).

Physical state: white powder

TLC: R_{f}=0.35 (20% ethyl acetate in hexanes, UV active, stains brown upon KMnO₄ staining) [α]²⁵_{D}: −55.4 (c=1.0, CHC13)

¹H-NMR (600 MHz, CDCl₃) δ 9.89-9.59 (m, 1H), 7.28-7.24 (m, 2H), 7.21-7.14 (m, 1H), 7.14-6.98 (m, 2H), 5.12-4.75 (m, 1H), 4.59 (d, J=5.7 Hz, 1H), 4.52 (d, J=16.4 Hz, 1H), 4.37 (d, J=16.4 Hz, 1H), 3.79-3.47 (m, 4H), 3.40-3.30 (m, 1H), 2.23-2.02 (m, 3H), 2.01-1.83 (m, 2H), 1.63-1.56 (m, 1H), 1.51 (s, 18H), 0.83 (s, 9H), −0.04 (s, 3H), −0.13 (s, 3H)

¹³C-NMR (150 MHz, CDCl₃) δ 204.8, 158.2, 155.7, 152.1, 139.5, 128.6, 126.7, 126.2, 99.4, 82.8, 57.4, 54.2, 53.0, 49.8, 48.1, 42.2, 30.9, 28.3, 25.8, 25.6, 20.3, 18.0, −4.7

HRMS (ESI-TOF): calc'd for C₃₄H₅₄N₂O₈Si [M+H]⁺: 647.3728, found: 647.3736

Compound 10

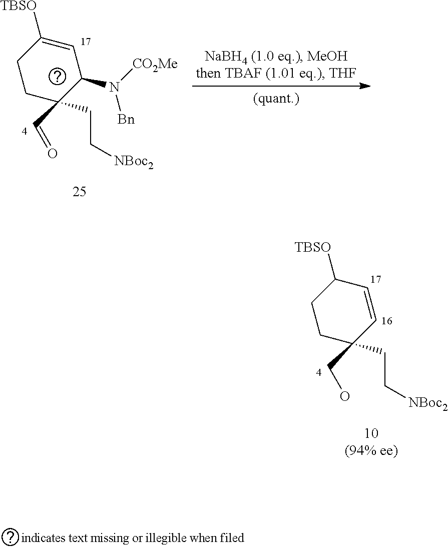

### Reduction

To a solution of compound 25 (81.4 g, 126 mmol) in a mixture of MeOH (400 ml) and DCM (100 ml) was added NaBH4 (4.7 g, 124 mmol) by portion at 0° C. The reaction was kept stirring for 1 hour, and was quenched by NH₄Cl (sat.) and H₂O. The mixture was extracted with DCM and dried over Na₂SO₄. The organic phase was concentrated under reduced pressure to afford the crude product, which is directly used for next step.

### E1cb

The crude product was dissolved in 500 ml THF, and TBAF (130 ml, 1M in THF) was added. The reaction mixture was stirred for 30 mins, and quenched by NH₄Cl (sat.) and H₂O. The mixture was extracted with DCM and dried over Na₂SO₄. The organic phase was concentrated under reduced pressure. The crude product was purified by chromatography (30% ethyl acetate in hexanes to 80% ethyl acetate in hexanes) to yield compound 10 (46 g, 126 mmol, quantitative yield).

*Note: Compound 10 is bench-stable.

Physical state: pale yellow oil

TLC: R_{f}=0.25 (30% ethyl acetate in hexanes, UV active, stains brown upon KMnO₄ staining)

[α]²⁵_{D}: +37.8 (c=1.0, CHC13)

¹H-NMR (600 MHz, CDCl₃) δ 6.82 (d, J=10.2 Hz, 1H), 6.01 (d, J=10.2 Hz, 1H), 3.71-3.61 (m, 3H), 3.56 (d, J=11.6 Hz, 1H), 2.93 (t, J=6.4 Hz, 1H), 2.55-2.38 (m, 2H), 1.95-1.76 (m, 4H), 1.51 (s, 18H)

¹³C-NMR (150 MHz, CDCl₃) δ 199.3, 154.3, 152.8, 129.5, 83.2, 66.8, 42.0, 40.3, 33.9, 33.1, 28.4, 28.2

HRMS (ESI-TOF): calc'd for C₁₉H₃₁NO₆ [M-Boc+2H]⁺: 270.1705, found: 270.1704

### Compound 11

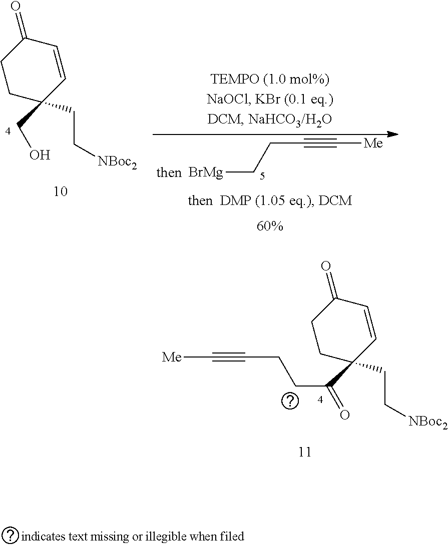

### Alcohol Oxidation

To a solution of compound 10 (16.1 g, 43.6 mmol) in DCM (60 ml) was added NaHCO₃ (15 g) and water (50 ml). The resulting mixture was added KBr (539 mg, 4.5 mmol) and TEMPO (68 mg, 0.44 mmol) at room temperature (open air). The reaction was cooled to 0° C., and a solution of NaOCl (6 wt %, 105 ml) was added slowly at the same temperature. The reaction mixture turned red immediately, and was warmed up to ambient temperature gradually by removing the ice bath.

After 1 hour, the reaction mixture was quenched by sat. Na₂S₂O₃ aq., and was extracted with DCM. The organic phase was dried over MgSO₄ and concentrated under reduced pressure. The product (ca. 16 g) was used directly to next step without purification.

### Grignard Reagent Preparation³

To a suspension of magnesium (3.1 g, 129 mmol) and LiCl (6.0 g, 141 mmol) in THF (29 ml), DIBAL-H (1.3 ml, 1.0 M in toluene) was dropwise subsequently under argon at 0° C.

Meanwhile, a solution of 3-pentynyl bromide (19.0 g, 129 mmol, in 72.8 ml THF) was prepared at room temperature. A small portion of this stock solution (8.7 ml) was added to the above suspension at 0° C. to initiate the Grignard reaction. After 15 minutes, the rest of the stock solution was added slowly. The residual bromide was rinsed by THE for three times (34 ml overall). The reaction mixture was kept stirring vigorously at 0° C. for 30 minutes and was warmed up to ambient temperature for another 30 minutes. The Grignard reagent was obtained as 0.54 M (theo. 0.95 M) indicated by titration with iodine.

### Grignard Addition

To a solution of the aldehyde (16 g, 43.6 mmol, in 320 ml THf) at −78° C. was added the freshly prepared Grignard reagent (110 ml, 59.4 mmol) dropwise in 1 hour (under argon). The reaction mixture was kept stirring at the same temperature for an extra hour, and AcOH (10 ml) was added to quench the reaction. The resulting solution was warmed up to room temperature gradually, and brine and sat. NaHCO₃ aq. was added. The mixture was extracted with ethyl acetate and dried over MgSO₄. The obtained organic phase was concentrated under reduced pressure to obtain the crude product, which was used in next step without purification.

### Alcohol Oxidation

The crude product obtained from Grignard addition was dissolved in DCM (100 ml, open air), and was cooled to 0° C. NaHCO₃ (10 g) was added to the solution, followed by Dess-martin periodinane (19.0 g, 44.8 mmol). The reaction mixture was warmed to ambient temperature. After 1 hour, the reaction mixture was diluted with DCM (ca. 50 ml), and was quenched by sat. Na₂S₂O₃ aq. and sat. NaHCO₃ aq. The resulting mixture was kept stirring for 45 minutes. The mixture was extracted with DCM and dried over MgSO₄. The organic phase was concentrated under reduced pressure. The crude product was purified by chromatography (5% EtOAc in hexanes to 20% EtOAc in hexanes) to yield compound 11(11.3 g, 26.1 mmol, 60%).

*Note: Compound 11 is bench-stable.

Physical state: light yellow oil

TLC: R_{f}=0.60 (30% ethyl acetate in hexanes, UV active, stains brown upon KMnO₄ staining)

[α]²⁵_{D}: −32.2 (c=1.0, CHC13)

¹H-NMR (600 MHz, CDCl₃) δ 7.01 (d, J=10.3 Hz, 1H), 6.09 (d, J=10.3 Hz, 1H), 3.60-3.45 (m, 2H), 2.81-2.68 (m, 2H), 2.54-2.35 (m, 5H), 2.06-1.93 (m, 3H), 1.72, (s, 3H), 1.50 (s, 18H)

¹³C-NMR (150 MHz, CDCl₃) δ 207.5, 198.3, 152.4, 150.1, 130.6, 83.0, 77.6, 76.6, 52.4, 42.1, 38.8, 36.5, 34.8, 29.8, 28.3, 13.6, 3.6

HRMS (ESI-TOF): calc'd for C₂₄H₃₅NO₆ [M+Na]⁺: 456.2362, found: 456.2350

### Compound 12

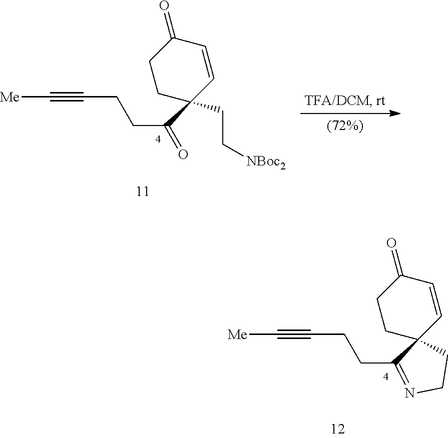

To a solution of compound 11 (8.8 g, 20.3 mmol) in DCM (62.8 ml) was added TFA (31.4 ml) at room temperature. The resulting red solution was stirred for 1.5 hours at the same temperature. The reaction mixture was added carefully (using dropping funnel) to a mixture of Na₂CO₃ (64 g, in 120 ml H₂O) and 4 M NaOH (24 ml), then stirred vigorously for 24 hours. The mixture was extracted with Et20 (for the first time) and DCM (for the rest of times). The organic layer was dried over MgSO₄, and was concentrated under reduced pressure. The crude product was purified by chromatography (DCM to 30% EtOAc+1% TEA in DCM) to yield compound 12 (3.14 g, 14.6 mmol, 72%).

*Note: Compound 12 is unstable for long-term storage (gradual decomposition even took place after 1 week at −20° C.), should be used as soon as possible.

Physical state: sticky orange oil

TLC: R_{f}=0.37 (pure EtOAc, highly UV active, stains brown upon KMnO₄ staining)

[α]²⁵_{D}: −155.8 (c=0.5, CHCl₃)

¹H-NMR (600 MHz, CDCl₃) δ 6.62 (dd, J=10.1, 1.6 Hz, 1H), 6.05 (d, J=10.1 Hz, 1H), 3.96 (dddt, J=16.0, 8.6, 3.8, 1.9 Hz, 1H), 3.81 (dtt, J=15.6, 7.6, 2.4 Hz, 1H), 2.58-2.48 (m, 4H), 2.48-2.34 (m, 2H), 2.20-2.09 (m, 2H), 1.96 (dddd, J=13.0, 8.7, 7.7, 1.0 Hz, 1H), 1.86 (dtd, J=13.5, 4.9, 1.7 Hz, 1H), 1.75 (t, J=2.5 Hz, 3H)

¹³C-NMR (150 MHz, CDCl₃) δ 198.3, 178.0, 152.0, 129.7, 78.4, 76.2, 58.3, 55.7, 35.2, 30.1, 29.3, 16.0, 3.7

HRMS (ESI-TOF): calc'd for C₁₄H₁₇NO [M+H]⁺: 216.1388, found: 216.1382

### Compound 26 (Ref. 5)

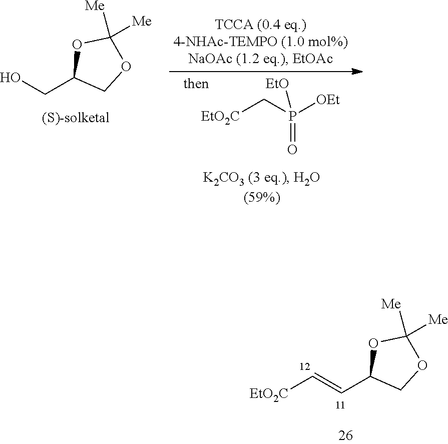 [SMILES: CC1(C)OC[C@@H](CO)O1 ; CCOC(=O)CP(=O)(OCC)OCC ; CCOC(=O)/C=C/[C@@H]1COC(C)(C)O1]

To a solution of (S)-solketal (50.0 g, 378 mmol) in EtOAc (500 ml) was added NaOAc (37 g, 451 mmol) and 4-NHAc-TEMPO (0.81 g, 3.8 mmol) under argon. TCCA (35 g, 151 mmol) was added by portions at 0° C. The mixture was stirring at the same temperature for 1.5 h, then filtered by Buchner funnel. The residue was rinsed with EtOAc, and the resulting filtrate was added water (190 ml), K₂CO₃ (157 g, 1.14 mol), and triethyl phosphonoacetate (81.5 ml, 92.1 g, 411 mmol) at 0° C. The reaction mixture was stirred overnight at ambient temperature, and a mixture of Et20 and water was added. The organic layer was separated and the aqueous phase was extracted with Et20. The combined organic layer was dried over MgSO₄ and concentrated under reduced pressure (100 mbar, 25° C.). The residual was collected and purified by chromatography (20% Et20 in pentane) to yield compound 26 (44.8 g, 224 mmol, 59%).

Physical state: colorless oil

TLC: R_{f}=0.40 (10% EtOAc in hexanes, UV active, stains brown upon KMnO₄ staining)

[α]²⁵_{D}: −36.4 (c=1.0, CHC13)

¹H-NMR (600 MHz, CDCl₃) δ 6.88 (dd, J=15.6, 5.6 Hz, 1H), 6.10 (dd, J=15.6, 1.4 Hz, 1H), 4.66 (tdd, J=7.0, 5.6, 1.4 Hz, 1H), 4.26-4.14 (m, 3H), 3.68 (dd, J=8.3, 7.0 Hz, 1H), 1.48-1.43 (m, 3H), 1.41 (d, J=0.8 Hz, 3H), 1.29 (t, J=7.1 Hz, 3H)

¹³C-NMR (150 MHz, CDCl₃) δ 166.2, 110.4, 75.1, 69.0, 60.8, 26.6, 25.9, 14.4

HRMS (ESI-TOF): calc'd for C₁₀ Hi604 [M+Na]⁺: 223.0941, found: 223.0942

### Compound 27 (Ref. 6)

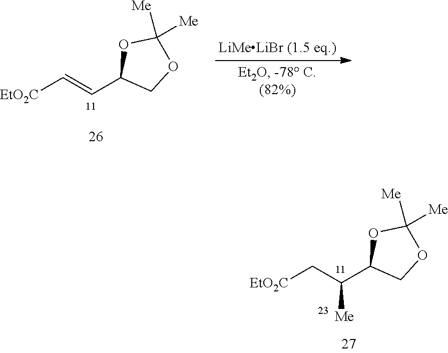 [SMILES: CCOC(=O)/C=C/[C@@H]1COC(C)(C)O1 ; CCOC(=O)C[C@H](C)[C@@H]1COC(C)(C)O1]

To a solution of compound 26 (21.0 g, 104 mmol) in Et20 (550 ml) was added MeLi LiBr (1.6 M in Et20, 71.5 ml, 157 mmol) dropwise via syringe pump under −78° C. over 2.5 h. Upon completion, the solution was quenched by MeOH (10 ml) and sat. NaHCO₃ aq. at −78° C., then warmed to rt slowly. The resulting mixture was extracted with Et20 and dried over MgSO₄. The solvents were removed under reduced pressure, and the crude product was purified by chromatography (0% to 20% EtOAc in hexanes) to obtain compound 27 (18.5 g, 85.5 mmol, 82%).

Physical state: colorless oil

TLC: R_{f}=0.47 (20% EtOAc in hexanes)

[α]²⁵_{D}: −9.60 (c=1.0, CHCl₃)

¹H-NMR (600 MHz, CDCl₃) δ 4.14 (q, J=7.2 Hz, 2H), 4.05-3.96 (m, 2H), 3.66-3.61 (m, 1H), 2.40 (dd, J=14.9, 5.0 Hz, 1H), 2.24-2.17 (m, 1H), 2.13 (dd, J=14.9, 8.8 Hz, 1H), 1.25 (s, 3H), 1.22 (s, 3H), 1.26 (t, J=7.2 Hz, 3H), 1.00 (d, J=6.8, 3H)

¹³C-NMR (150 MHz, CDCl₃) δ 172.8, 109.1, 79.0, 67.0, 60.6, 37.8, 33.2, 26.6, 25.4, 15.6, 14.4

HRMS (ESI-TOF): calc'd for C₁₁H₂₀O₄ [M+Na]⁺: 239.1259, found: 239.1254

### Compound 28

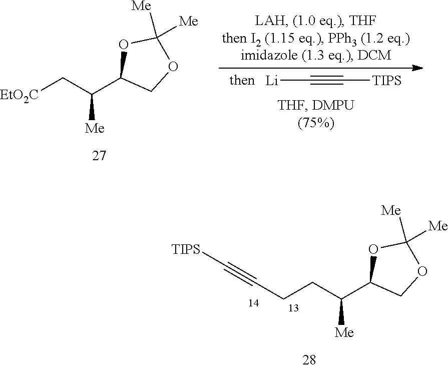

To a solution of compound 27 (10.4 g, 48.1 mmol) in THF (90 ml) was added LAH (1.83 mg, 48.2 mmol) by portions at 0° C. The ice bath was removed, then the reaction mixture was allowed to stir at ambient temperature for 2 h. Upon completion, EtOAc (100 ml) was added slowly, then water (2 ml) was added to quench the remaining LAH. After stirring for 30 mins, sat. Rochelle's salt aq. was added to this mixture and stirred overnight. After separating the organic layer, the aqueous phase was extracted with EtOAc. The combined organic layer was dried over MgSO₄ and concentrated-under reduced pressure to yield the crude alcohol. The alcohol obtained from above was dissolved in DCM (50 ml) under argon, imidazole (4.25 g, 62.5 mmol) and PPh₃ (15.1 g, 57.7 mmol) was added. To this mixture, 12 (14.0 g, 55.3 mmol) was added slowly. The red color faded since all 12 was consumed. Upon completion (indicated by TLC), methanol was added to quench the excess iodine, followed by blowing air to this solution to quench the excess PPh₃ (normally takes 30 mins). The volatiles were then removed by reduced pressure, and pentane (150 ml) was added and stirred vigorously (sonicate if necessary). After 1 h, the solids were removed by Buchner funnel, and the residue was washed with pentane for couple of times. Subsequently, the filtrate was collected and the pentane was removed under reduced pressure to give the crude iodide (contains ~6% TPPO).

In another flame-dried round bottom flask, TIPS acetylene (9.65 g, 52.9 mmol) was dissolved in THF (86 ml), and n-BuLi (2.5 M in hexanes, 19.2 ml, 48.1 mmol) was added dropwise under argon. The mixture was then stirred at 0° C. for 30 mins, and cooled back to −78° C. DMPU (12 ml) was added to this solution in one portion. Subsequently, a solution of the aforementioned iodide (in 20 ml THF) was added dropwise to the freshly prepared lithium acetylide. The mixture was stirred at ambient temperature overnight, and quenched with water. The aqueous phase was extracted with Et20 and combined. The combined organic phase was rinsed with water for 3 times to remove the residual DMPU, and dried over MgSO₄. After removal of the solvents, the residue was purified with chromatography (hexanes to 5% EtOAc in hexanes) to give compound 28 (12.2 g, 36.1 mmol, 75%) Physical state: colorless oil

TLC: R_{f}=0.21 (30% DCM in hexanes, stains brown upon KMnO₄ staining)

[α]²⁵_{D}: −16.5 (c=1.0, CHCl₃)

¹H-NMR (600 MHz, CDCl₃) δ 4.02 (dd, J=7.6, 6.3 Hz, 1H), 3.92 (q, J=6.3 Hz, 1H), 3.64 (t, J=7.6 Hz, 1H), 2.35 (ddd, J=17.0, 7.5, 5.7 Hz, 1H), 2.27 (ddd, J=17.0, 8.2, 7.3 Hz, 1H), 1.87-1.75 (m, 1H), 1.61-1.54 (m, 1H), 1.39 (s, 3H), 1.35 (s, 3H), 1.36-1.29 (m, 1H), 1.10-1.00 (m, 21H), 0.99 (d, J=6.6 Hz, 3H)

¹³C-NMR (150 MHz, CDCl₃) δ 108.9, 108.6, 80.8, 79.9, 67.9, 35.6, 32.2, 26.7, 25.7, 18.8, 17.8, 15.0, 11.5

HRMS (ESI-TOF): calc'd for C₂₀H₃₈O₂Si [M+H]⁺: 339.2719, found: 339.2711

### Compound 29

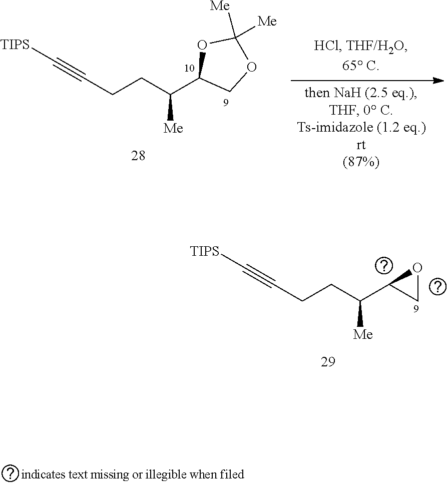

A solution of compound 28 (18.5 g, 54.5 mmol) in a mixture of HCl (3M, 50 ml) and THF (100 ml) was heated at 75° C. for 12 h. The mixture was then cooled down to room temp, and sat. NaHCO₃ aq. was slowly added to neutralize the HCl. The mixture was extracted with ether and the organic phase was dried over MgSO₄. The crude was obtained from concentrating under reduced pressure and was used directly.

NaH (5.4 g, 60 wt % in mineral oil, 135 mmol) was suspended in THF (100 ml) under argon atmosphere. This mixture was then cooled down to 0° C. To this solution, the crude obtained above (dissolved in 20 ml THF) was added dropwise. Upon completion, ice bath was removed to allow the mixture to warm up. The mixture was then stirred under ambient temperature for 1.5 h, and tosyl imidazole (14.4 g, 65 mmol) was added by portion. The resulting mixture was stirred vigorously at the same temperature for 45 min. Upon completion, water was added in and the mixture was stirred for 15 mins. The organic layer was separated and the aqueous phase was extracted with Et20 for 3 times. The combined organic phase was dried over MgSO₄ and concentrated. The crude product was purified by chromatography (hexanes to 30% DCM in hexanes) to yield epoxide 29 (13.3 g, 47.4 mmol, 87%).

Physical state: colorless oil

TLC: R_{f}=0.33 (40% DCM in hexanes, stains brown upon KMnO₄ staining)

[α]²⁵_{D}: +13.3 (c=1.0, CHCl₃)

¹H-NMR (600 MHz, CDCl₃) δ 2.77 (dd, J=5.0, 3.2 Hz, 1H), 2.70 (dt, J=6.8, 3.2 Hz, 1H), 2.59 (dd, J=5.0, 3.2 Hz, 1H), 2.39-2.26 (m, 2H), 1.64 (tt, J=11.3, 4.7 Hz, 1H), 1.56-1.47 (m, 2H), 1.13-0.96 (m, 24H)

¹³C-NMR (150 MHz, CDCl₃) δ 108.4, 80.9, 56.7, 47.1, 35.3, 32.8, 18.8, 17.9, 16.9, 11.4

HRMS (ESI-TOF): calc'd for C₁₇H₃₂OSi [M+H]⁺: 281.2301, found: 281.2296

### Compound 30

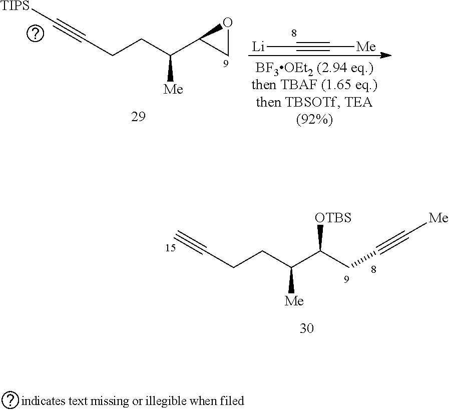

A solution of n-BuLi (73.0 ml, 182 mmol in hexanes) in THE (300 ml) was cooled to −78° C. under argon. To this solution, a balloon of propyne was added via needle, and the solution began to absorb the gas. Upon completion (the solution turned cloudy), the balloon was removed. The generated mixture was allowed to stir at −78° C. for 30 min.

To this freshly prepared lithium acetylide, a solution of compound 29 (17.0 g, 60.6 mmol) in THF (30 ml) was added slowly at the same temperature. BF3 etherate (22.0 ml, 178 mmol) was added subsequently, and the reaction mixture was allowed to stir at −78° C. for 1 h. The reaction was quenched with sat. NaHCO₃ aq. and diluted with Et20, then stirred for 1 h. The mixture was extracted with EtOAc and the organic layer was dried over MgSO₄. The solvents were removed under vacuum and the crude product was directly used for next transformation.

The crude product obtained from above was added TBAF (100 ml, 1 M in THF, 100 mmol) under argon. The resulting mixture was stirred at 45° C. overnight. Upon completion, sat. NH₄Cl aq. and EtOAc was added. The mixture was extracted with EtOAc and dried over MgSO₄. After removing solvents under reduced pressure, the crude was dissolved in DCM (120 ml), then added TEA (18.2 g, 25.1 ml, 180 mmol). To this solution, TBSOTf (31.7 g, 27.6 ml, 120 mmol) was slowly added under room temperature. The mixture was stirred at ambient temperature for 30 mins, the heated to reflux for 1 h. Upon completion, sat. NaHCO₃ aq. was added and the mixture was extracted with DCM. The combined organic layer was dried over MgSO₄ and concentrated. The crude was purified by chromatography (0-5% EtOAc in hexanes) to yield compound 30 (15.5 g, 55.7 mmol, 92%).

Physical state: colorless oil

TLC: R_{f}=0.50 (5% EtOAc in hexanes, stains brown upon KMnO₄ staining)

[α]²⁵_{D}: −10.5 (c=1.0, CHCl₃)

¹H-NMR (600 MHz, CDCl₃) δ 3.71 (td, J=6.7, 2.8 Hz, 1H), 2.30-2.22 (m, 2H), 2.18 (dtd, J=16.9, 7.8, 2.6 Hz, 1H), 1.93 (t, J=2.6 Hz, 1H), 1.92-1.85 (m, 1H), 1.77 (t, J=2.6 Hz, 3H), 1.67 (dtd, J=13.3, 7.9, 5.3 Hz, 1H), 1.38 (dtd, J=13.3, 8.4, 6.0 Hz, 1H), 0.88 (s, 9H), 0.85 (d, J=6.8 Hz, 3H), 0.08 (s, 3H), 0.06 (s, 3H)

¹³C-NMR (150 MHz, CDCl₃) δ 84.8, 77.2, 76.8, 74.5, 68.3, 36.6, 32.3, 26.0, 25.0, 18.3, 16.7, 13.0, 3.70, −4.1, −4.6

HRMS (ESI-TOF): calc'd for C₁₇H₃₀OSi [M+H]⁺: 279.2139, found: 279.2142

### Compound 13

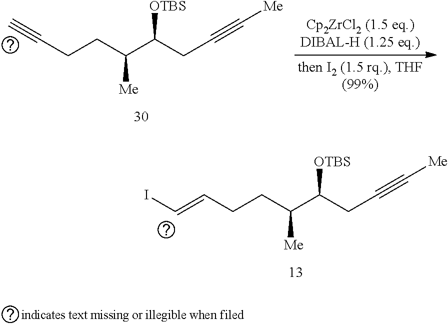 [SMILES: C#CCC[C@H](C)[C@H](CC#CC)O[Si](C)(C)C(C)(C)C ; CC#CC[C@H](O[Si](C)(C)C(C)(C)C)[C@@H](C)CC/C=C/I]

To a solution of Cp₂ZrCl₂(13.6 g, 46.5 mmol) in THE (100 ml) was added DIBAL-H (38.6 ml, 38.6 mmol, 1.0 M in hexanes) dropwise at 0° C. A white precipitation was formed. The mixture was allowed to stir for 1 hour, and a solution of compound (8.58 g, 30.8 mmol) in THF (10 ml) was added slowly. The reaction mixture (cloudy) was warmed up to room temperature slowly, and kept stirring overnight. A solution of 12(12.0 g, 47.2 mmol) in THF (20 ml) was added at 0° C., the resulting dark solution was stirred for 1 h at the same temperature. The reaction was quenched with sat. Na₂S₂O₃ aq., and filtered to remove undissolved impurities (if necessary). The obtained mixture was extracted with Et20, and the organic phase was dried over MgSO₄ and concentrated under reduced pressure to give crude compound. Pure compound 13 (12.4 g, 30.4 mmol, 99%) was obtained by chromatography (2% EtOAc in hexanes).

*Note: Compound 13 is highly stable and can be stored as neat (at 5 Q) for a couple of months.

Physical state: pale yellow oil

TLC: R_{f}=0.44 (3% EtOAc in hexanes, UV active, stains brown upon KMnO₄ staining)

[α]²⁵_{D}: −13.6 (c=1.0, CHCl₃)

¹H-NMR (600 MHz, CDCl₃) δ 6.51 (dt, J=14.3, 7.2 Hz, 1H), 5.99 (dd, J=14.3, 1.5 Hz, 1H), 3.68 (td, J=6.8, 2.8 Hz, 1H), 2.29-2.18 (m, 2H), 2.17-1.99 (m, 2H), 1.78 (t, J=2.6 Hz, 3H), 1.77-1.70 (m, 1H), 1.55-1.46 (m, 1H), 1.28-1.18 (m, 1H), 0.88 (s, 9H), 0.83 (d, J=6.8 Hz, 3H), 0.07 (s, 3H), 0.04 (s, 3H)

¹³C-NMR (150 MHz, CDCl₃) δ 146.8, 77.3, 76.7, 74.6, 74.4, 36.4, 34.1, 32.0, 26.0, 25.0, 18.3, 13.3, 3.8, −4.1, −4.5

HRMS (ESI-TOF): calc'd for C₁₇H₃₁IOSi [M+H]⁺: 407.1267, found: 407.1260

### Compound 7

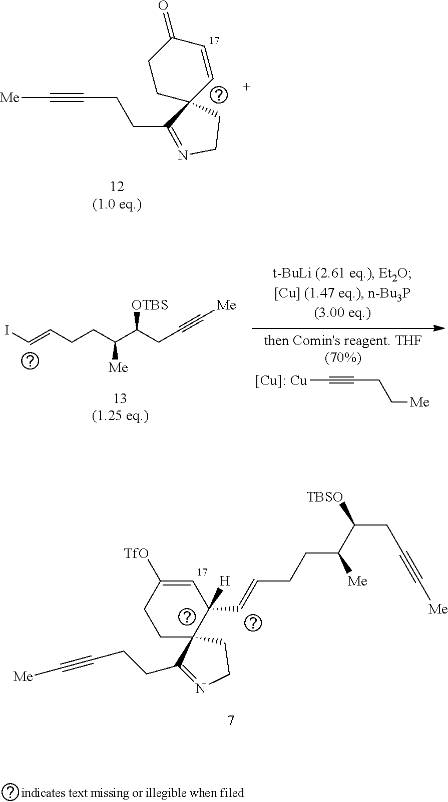

### Lithium-Halogen Exchange

To a solution of compound 13 (7.42 g, 18.3 mmol, in 46 ml Et20) at −78° C., t-BuLi (23.8 ml, 1.6 M in pentane, 38.1 mmol) was added dropwise. A bright yellow solution was formed immediately, followed by a formation of precipitation. Reaction was kept stirring under −78° C. for at least 2.5 hours to give the solution of vinyllithium.

### Copper(I)-Phosphine Complex Preparation

At the meantime, [Cu](2.86 g, 21.5 mmol) was suspended in THE (16 ml) under argon. To this suspension, n-Bu₃P (10.9 ml, 8.82 g, 43.6 mmol) was added dropwise. Dissolving of the yellow participate took place and finished in 15-30 minutes.

### Transmetallation and Conjugate Addition

To the generated vinyllithium solution at −78° C., the prepared copper-phosphine complex was added dropwise in 5 mins. After addition, the orange mixture was stirred for 1 h at −78° C.

A solution of compound 12 (3.14 g, 14.6 mmol, in 10 ml THF) was added to the above solution dropwise at −78° C. The resulting mixture was kept stirring at the same temperature for 40 mins. Then a solution of Comins' reagent (8.59 g, 21.9 mmol, in 20 ml THF) was added and the resulting solution was warmed to 0° C. The reaction mixture was stirred at 0° C. for 1 hour, and quenched by 5% NH₃ aq., Na₂CO₃, and blew with air. The resulting mixture was stirred vigorously for 45 mins, and extracted with ether for three times. The organic layer was dried over MgSO₄. The solvent was removed under reduced pressure and the resulting residue was purified by gradient chromatography (20% Et20 in hexanes to 40% Et₂O+1% TEA in hexanes) to yield compound 7 (6.41 g, 10.2 mmol, 70%).

*Note: Compound 7 gradually decomposed in crude, it'll be better to purify the crude product as soon as possible (in the same day). RCAM step will not work if impurity exists in this step.

Physical state: pale yellow oil

TLC: R_{f}=0.58 (50% Et20 in hexanes, stains brown upon KMnO₄ staining)

[α]²⁵_{D}: −53.1 (c=1.0, CHCl₃)

¹H-NMR (600 MHz, CDCl₃) δ 5.68 (d, J=2.0 Hz, 1H), 5.46 (dt, J=14.3, 6.8 Hz, 1H), 5.21 (ddt, J=14.3, 7.44, 1.49 Hz, 1H), 3.85-3.77 (m, 1H), 3.67 (td, J=6.96, 2.80 Hz, 1H), 3.52 (tdd, J=10.3, 8.3, 4.1 Hz, 1H), 3.17-3.06 (m, 1H), 2.56-2.51 (m, 2H), 2.50-2.34 (m, 4H), 2.28-2.18 (m, 2H), 2.10-1.93 (m, 3H), 1.93-1.86 (m, 1H), 1.71-1.65 (m, 1H), 1.81-1.73 (m, 6H), 1.65-1.53 (m, 2H), 1.46-1.39 (m, 1H), 1.21-1.12 (m, 1H), 0.88 (s, 9H), 0.81 (d, 0.1=6.8 Hz, 3H), 0.07 (s, 3H), 0.04 (s, 3H)

¹³C-NMR (150 MHz, CDCl₃) δ 177.8, 148.9, 134.4, 126.3, 120.6, 118.7 (q, J_{C-F}=316 Hz), 79.0, 77.1, 76.9, 76.0, 74.7, 58.8, 56.7, 43.1, 37.0, 33.2, 31.5, 30.9, 30.1, 29.0, 26.0, 25.5, 25.1, 18.3, 15.9, 13.2, 3.7, −4.0, −4.5

¹H-COSY, ¹H-¹³C-HSQC, ¹H-¹³C-HMBC, NOESY spectra are available

HRMS (ESI-TOF): calc'd for C₃₂H₄₈F₃NO₄SSi [M+H]⁺: 628.3104, found: 628.3093

### Compound 31

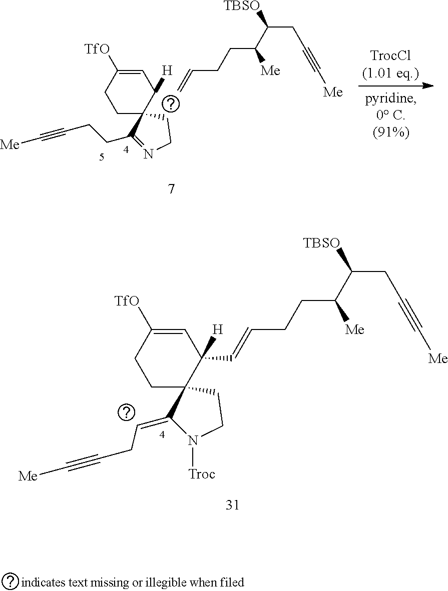

To a solution of freshly prepared compound 7 (1.66 g,) in DCM (16.0 ml) was added DMAP (1.8 g,) and TrocCl ( ), the resulting solution was then heated to reflux and

Physical state: colorless oil

TLC: R_{f}=0.44 (50% DCM in hexanes, UV active, stains brown upon KMnO₄ staining)

[α]²⁵_{D}: +82.1 (c=1.0, CHC13)

¹H-NMR (600 MHz, CDCl₃) δ 5.71 (d, J=4.7 Hz, 1H), 5.54 (dt, J=15.4, 6.7 Hz, 1H), 5.33 (dd, J=15.4, 8.0 Hz, 1H), 5.00 (s, 1H), 5.20-4.41 (br s, 2H), 3.73-3.64 (m, 2H), 3.62-3.50 (m, 1H), 3.01-2.87 (m, 2H), 2.87-2.70 (m, 1H), 2.43-2.29 (m, 1H), 2.29-2.18 (m, 3H), 2.08 (ddt, J=15.5, 10.9, 6.0 Hz, 1H), 2.04-1.95 (m, 1H), 1.81 (dt, J=13.3, 9.7 Hz, 1H), 1.77 (t, J=2.6 Hz, 3H), 1.76 (t, J=2.6 Hz, 3H), 1.74-1.65 (m, 1H), 1.63-1.50 (m, 3H), 1.45 (ddt, J=12.8, 9.5, 5.6 Hz, 1H), 1.24-1.16 (m, 1H), 0.87 (s, 9H), 0.82 (d, J=6.8 Hz, 3H), 0.07 (s, 3H), 0.03 (s, 3H).

¹³C-NMR (150 MHz, CDCl₃) δ 148.8, 141.0, 135.1, 127.5, 121.8, 120.3, 118.7 (q, J_{C-F}=320.4 Hz), 95.6, 77.1, 76.9, 75.1, 74.5, 45.7, 45.5, 45.0, 36.9, 33.3, 31.5, 30.7, 26.0, 25.1, 19.4, 18.3, 13.1, 3.7, 3.7, −4.0, −4.6

(a few carbons missed in ¹³C-NMR due to rotamers)

¹H-¹³C-HSQC, ¹H-¹³C-HMBC, NOESY spectra are available

HRMS (ESI-TOF): calc'd for C₃₅H₄₉C₁₃F₃NO₆SSi [M+H]⁺: 802.2140, found: 802.2124

### Compound 31

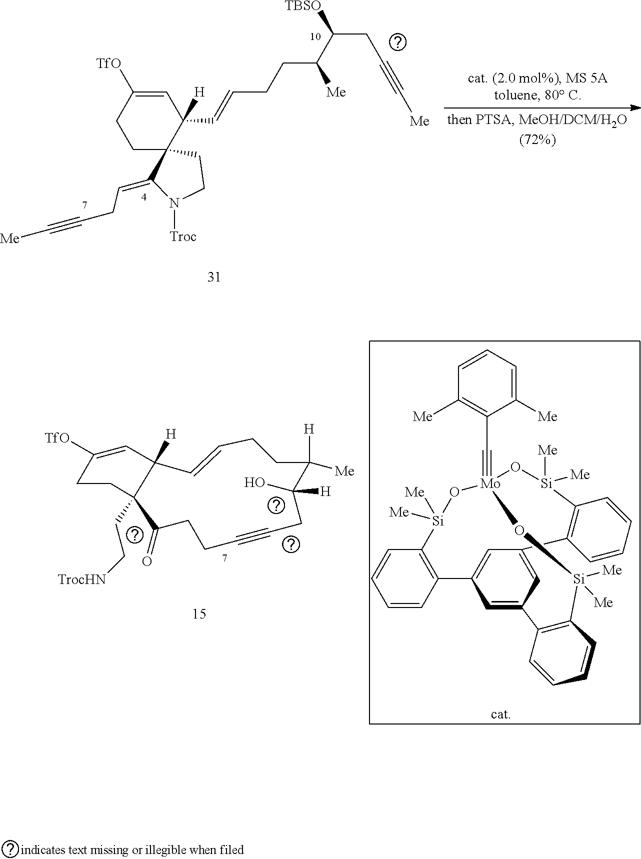

A 250 ml round bottom flask charged freshly prepared compound 31 (2.35 g, 2.93 mmol) was added MS 5 Å (7.01 g, 250 wt % to SM, dried at 210° C. under vacuum overnight) and cat. (43 mg, 0.058 mmol) in glovebox. Then the flask was taken out from glovebox, and toluene (140 ml) was added under argon. The mixture was pre-stirred for 5 mins, and heated to 80° C. After 45 mins, TLC (60% DCM in hexanes) indicated the full conversion. The reaction mixture was allowed to filter over a Celite® plug to remove the molecular sieves, and rinsed with DCM. The filtrate was concentrated under reduced pressure and gave a pale-yellow foam as crude product.

The crude product was dissolved in a mixture of DCM (2.4 ml), MeOH (24 ml) and H₂O (1.2 ml). To this solution, PTSA (lg, 5.3 mmol) was added, and the solution was heated to 50° C. overnight. The reaction mixture was quenched with sat. NaHCO₃ aq. and brine, and was extracted with DCM. The resulting organic phase was dried over MgSO₄ and concentrated under reduced pressure. The crude product was purified by chromatography (DCM to 20% Et20 in DCM) to yield compound 15 (1.38 g, 2.11 mmol, 72%).

*Note: Compound 15 is bench-stable.

Physical state: white solid

TLC: R_{f}=0.45 (20% Et20 in hexanes, stains brown upon KMnO₄ staining)

[α]²⁵_{D}: −41.4 (c=0.5, CHC13)

¹H-NMR (600 MHz, CDCl₃) δ 5.65 (ddd, J=15.3, 9.7, 4.7 Hz, 1H), 5.60 (d, J=2.3 Hz, 1H), 5.27 (dd, J=15.3, 8.6 Hz, 1H), 5.17 (t, J=5.8 Hz, 1H), 4.74 (d, J=12.0 Hz, 1H), 4.67 (d, J=12.0 Hz, 1H), 3.56-3.50 (m, 1H), 3.45 (s, 1H), 3.34-3.27 (m, 1H), 3.26-3.20 (m, 1H), 2.80 (ddd, J=18.8, 11.2, 3.0 Hz, 1H), 2.64-2.46 (m, 4H), 2.46-2.28 (m, 3H), 2.20-2.01 (m, 4H), 1.95 (dd, J=14.0, 5.8 Hz, 1H), 1.85 (dq, J=9.4, 6.6 Hz, 1H), 1.64 (ddd, J=14.0, 9.4, 5.0 Hz, 1H), 1.50-1.39 (m, 2H), 1.23-1.15 (m, 1H), 0.93 (d, J=6.6 Hz, 3H)

¹³C-NMR (150 MHz, CDCl₃) δ 209.9, 154.7, 147.6, 133.9, 126.5, 120.5, 118.6 (q, J_{C-F}=320 Hz), 95.7, 81.5, 75.8, 74.7, 73.0, 52.0, 46.2, 37.7, 35.7, 33.0, 32.3, 28.3, 28.0, 27.2, 25.6, 24.9, 13.6, 13.1

HRMS (ESI-TOF): calc'd for C₂₅H₃₁C₁₃F₃NO₇S [M+H]⁺: 652.0917, found: 652.0919

### Compound 16

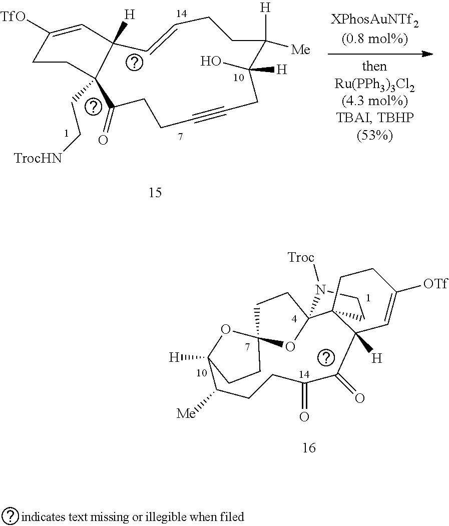

To a solution of compound 15 (3.02 g, 4.62 mmol, azeotrope with benzene to remove residual H₂O) in DCM (30 ml) was added XPhosAuNTf₂ (30.2 mg, 0.032 mmol) under argon. The mixture was refluxed for 2 hours. Upon completion (indicated by TLC), it was cooled to room temperature, and TEA (0.05 ml) was added to quench the gold catalyst. Subsequently, the solvent was removed under reduced pressure to give a white foam.

The resulting polycyclic compound was dissolved in MeCN/PhMe/H₂O (5:5:1, 112 ml), and TBAI (808 mg, 2.19 mmol) was added in one portion to give a clear solution. Ru(PPh₃)₃Cl₂ (191 mg, 0.2 mmol) was added to this solution subsequently. A gray-green mixture was generated. Subsequently, TBHP (5-6 M in decane, 10.9 ml) was added dropwise. The yellow green solution turned dark (ca. 15 mins, prevent light by aluminum foil since the product is sensitive to light). The reaction was allowed to stir at ambient temperature for 1.5 hours (open air, water bath to maintain a constant temperature). Upon completion, the reaction was diluted by Et20, and quenched by a mixture of water, solid NaHSO₃ and NaCl. The resulting cloudy mixture was stirred for 1 hours to give a good phase separation. After collecting the organic phase, the aqueous phase was extracted with Et20 for at 4 times. The combined organic phase was dried over MgSO₄, and concentrated under reduced pressure, The crude product was purified by chromatography (DCM to 5% Et20 in DCM) to yield compound 16 (1.67 g, 2.44 mmol, 52%).

*Note: Compound 16 is light-sensitive and very unstable in solvents (CHCls, benzene, etc.). It's necessary to remove the solvents for storage.

Physical state: yellow foam

TLC: R_{f}=0.45 (20% Et20 in hexanes, yellow under light, stains brown upon KMnO₄ staining)

[α]²⁵_{D}: +40.4 (c=1.0, MeOH)

¹H-NMR (600 MHz, CD₃CN) 6 5.57 (m, 1H), 4.82 (d, J=12.3 Hz, 1H), 4.70 (d, J=12.3 Hz, 1H), 4.48 (dt, J=4.9, 2.5 Hz, 1H), 3.78 (ddd, J=10.1, 6.1, 3.8 Hz, 1H), 3.68-3.53 (m, 1H), 3.47 (td, J=11.1, 7.1 Hz, 1H), 3.15-3.06 (m, 1H), 2.77 (dt, J=12.6, 10.0 Hz, 1H), 2.68 (dddd, J=13.6, 11.5, 9.7, 1.8 Hz, 1H), 2.63-2.54 (m, 1H), 2.38 (ddt, J=18.3, 5.3, 2.3 Hz, 1H), 2.32-2.20 (m, 3H), 2.02 (ddd, J=13.6, 8.6, 3.0 Hz, 1H), 1.96-1.93 (m, 1H), 1.88 (dd, J=13.2, 7.0 Hz, 1H), 1.81-1.73 (m, 1H), 1.72-1.64 (m, 3H), 1.63-1.53 (m, 2H), 0.83 (d, J=7.2 Hz, 3H)

¹³C-NMR (150 MHz, CD₃CN) 6 199.8, 199.0, 152.7, 150.6, 119.7 (q, J_{C-F}=320 Hz), 119.0, 117.9, 105.5, 96.9, 84.3, 75.0, 52.4, 45.2, 40.36, 37.6, 37.4, 37.0, 32.9, 29.1, 27.4, 25.4, 25.4, 24.1, 23.8, 18.9

HRMS (ESI-TOF): calc'd for C₂₅H₂₉C₁₃F₃NO₉S [M+H]⁺: 682.0659, found: 682.0668

### Compound 32

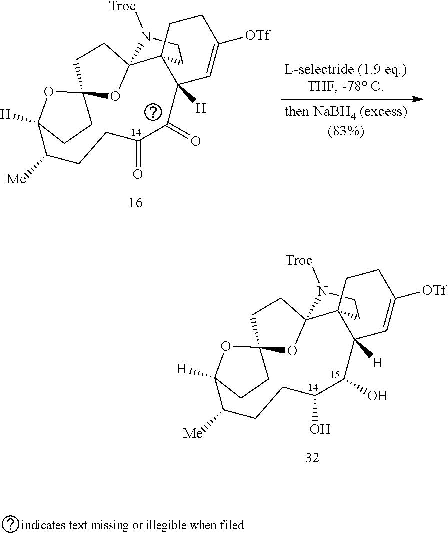

To a solution of compound 16 (2.43 g, 3.53 mmol) in THF (135 ml) was added L-selectride (1.0 M in THF, 6.7 ml) at −78° C. under argon. The yellow solution turned to colorless as completion (usually takes 30 mins). The reaction was quenched according to the following procedure:
i. Adding MeOH (3.8 ml) to the mixture at −78° C. and stirred at this temperature for 5 mins.
ii. The cooling bath was removed, and a mixture of H₂O (50 ml), EtOAc (10 ml) and Na₂CO₃ (3.0 g) was added subsequently. The resulting mixture was allowed to stir for 5 mins.
iii. A solution of H₂O₂ (50 wt % in H₂O, 2 ml) was added dropwise, and the mixture was stirred for 60 mins at room temperature.

After work up, the mixture was extracted with EtOAc for 3 times, and the combined organic layer was dried over MgSO₄. After concentrating under reduced pressure, the residue was dissolved in MeOH (100 ml) and cooled to 0° C. NaBH4 (excess, around 500 mg) was added by portion at this temperature. The reaction was stirred for 1 h. Sat. NH₄C₁ aq. was added after full conversion, and the mixture was extracted with DCM for 3 times. The combined organic phase was dried over MgSO₄ and concentrated, the crude product was purified by chromatography (5% EtOAc in DCM to 15% EtOAc in DCM) to yield compound 32 (2.03 g, 83%).

Physical state: white foam

TLC: R_{f}=0.23 (30% EtOAc in hexanes, stains brown upon KMnO₄ staining)

[α]²⁵ D: +17.9 (c=1.0, CHCl₃)

¹H-NMR (500 MHz, CDCl₃) δ 5.86 (s, 1H), 4.81 (d, J=12.0 Hz, 1H), 4.64 (d, J=12.0 Hz, 1H), 4.06 (td, J=8.9, 8.3, 4.0 Hz, 1H), 3.92-3.82 (m, 1H), 3,60 (t, J=10.4 Hz, 1H), 3.56-3.10 (m, 3H), 3.03-2.81 (m, 1H), 2.57-2.22 (m, 5H), 2.11-1.74 (m, 10H), 1.73-1.52 (m, 4H), 0.86 (d, J=7.2 Hz, 3H)

¹³C-NMR (125 MHz, CDCl₃) δ 152.3, 149.3, 124.0, 118.7 (q, J_{C-F}=320 Hz), 116.2, 106.5, 96.0, 86.2, 81.4, 75.0, 74.6, 66.0, 49.7, 44.8, 38.0, 36.4, 35.8, 34.5, 30.0, 30.0, 28.7, 26.6, 25.1, 24.1, 18.1

HRMS (ESI-TOF): calc'd for C₂₅H₃₃Cl₃F₃NO₉S [M−OH]⁺: 668.0866, found: 668.0869

### Compound 19

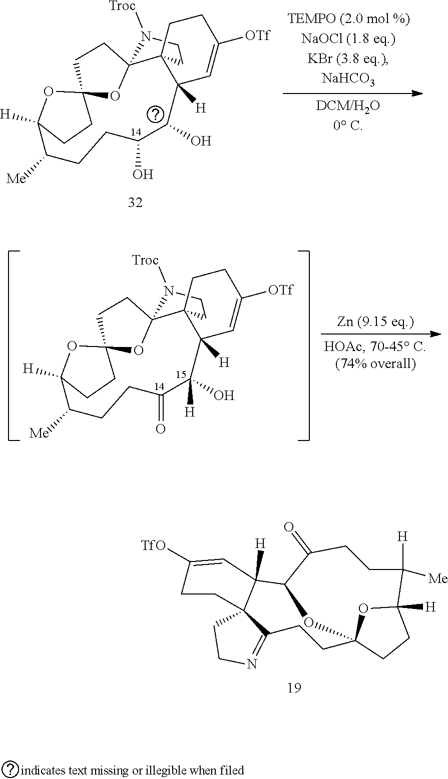

To a solution of compound 32 (1.95 g, 2.84 mmol) in DCM (37 ml) was added TEMPO (55.5 mg, 0.059 mmol), KBr (1.3 g, 10.9 mmol) and sat. NaHCO₃ aq. (9.3 ml), then cooled to 0° C. The mixture was then stirred for 5 mins and a solution of NaOCl (6%, ca. 0.8 M, 5.1 ml) was added dropwise. The resulting red biphasic solution was stirred at the same temperature for 3.5 h, then quenched by adding sat. Na₂S₂O₃ aq. The yellow mixture turned colorless in a few seconds, followed by extraction with DCM for couple of times. The combined organic layer was dried over MgSO₄ and concentrated under reduced pressure to give a white foam (C-14 oxidized product).

The crude compound obtained above was mixed with zinc powder (1.7 g, 26 mmol), then dissolved in a mixture of AcOH (70 ml) and H₂O (23 ml), heated at 70° C. under argon. The mixture was stirred for 3 h at the same temperature, then cooled down to 45° C. After stirring at 45° C. for 2 h, the mixture was cooled down to ambient temperature, diluted with EtOAc, and dropped into a solution of K₂CO₃ (70 g in 100 ml H₂O). After quenching the acetic acid, the biphasic mixture was extracted with DCM for 3 times, and dried over MgSO₄. After removal of the solvents, the crude product was purified with chromatography (EtOAc to 10% MeOH+1% TEA in EtOAc) to give compound 19 (1.03 g, 2.10 mmol, 74%).

Physical state: pale yellow powder

TLC: R_{f}=0.23 (3% MeOH in EtOAc, stains brown upon KMnO₄ staining)

[α]²⁵_{D}: −35.6 (c=0.5, CHC13)

¹H-NMR (600 MHz, CDCl₃) δ 5.09 (q, J=1.7 Hz, 1H), 4.29 (dt, J=10.3, 6.2 Hz, 1H), 4.04 (d, J=12.0 Hz, 1H), 4.00-3.94 (m, 1H), 3.89-3.81 (m, 1H), 3.65-3.58 (dtt, J=17.2, 6.9, 3.6 Hz, 1H), 2.74-2.56 (m, 3H), 2.55-2.43 (m, 4H), 2.38-2.28 (m, 1H), 2.19-2.07 (m, 2H), 2.00-1.80 (m, 4H), 1.79-1.68 (m, 3H), 1.49 (dt, J=12.6, 6.3 Hz, 1H), 1.43 (dd, J=14.6, 8.1 Hz, 1H), 0.83 (d, J=7.1 Hz, 3H)

¹³C-NMR (150 MHz, CDCl₃) δ 206.4, 182.0, 148.4, 119.1, 118.6 (q, J_{C-F}=320 Hz), 110.7, 84.6, 75.9, 56.0, 53.6, 48.7, 40.4, 37.2, 36.5, 34.1, 28.6, 28.3, 27.0, 26.3, 24.8, 24.4, 17.7

HSQC AVAILABLE

HRMS (ESI-TOF): calc'd for C₂₂H₂₈F₃NO₆S: [M+H]⁺: 492.1668, found: 492.1663

### Compound 20

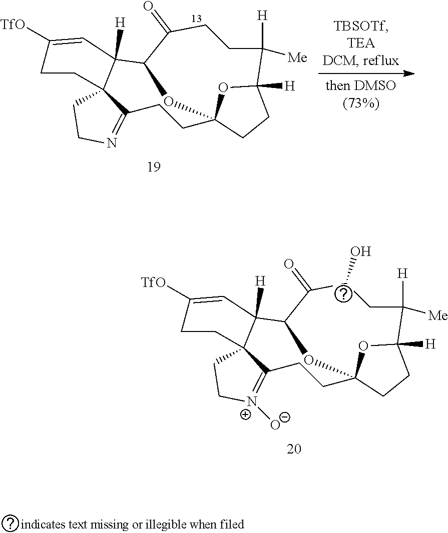

### Silylation

To a solution of compound 19 (505 mg, 1.03 mmol) in refluxing DCM (5.0 ml) was added TEA (1.07 ml, 777 mg, 7.68 mmol) and TBSOTf (0.75 ml, 862 mg, 3.26 mmol). The reaction mixture was then allowed to stir for 45 min. Upon completion, the mixture was rinsed with sat. NaHCO₃ aq., followed by extraction with DCM. The organic phase was dried over MgSO₄, filtered and concentrated to give crude silyl enol ether as product.

### DMDO Oxidation

The crude product from above was dissolved in DCM (3 ml), and added DMDO solution (freshly prepared, ca. 0.08 M in acetone, 50 ml) at 0° C. dropwise. After stirring at 0° C. for 1 h, the solvent was removed in vacuo. The residual was redissolved in acetone/H₂O (v/v=1:1, 20 ml) and added NaHCO₃ solid. Oxone® (550 mg) was added to this mixture, and the reaction was stirred at ambient temperature for 1 h. Upon completion, a mixture of H₂O and DCM was added. The aqueous phase was extracted with DCM for four times, and the organic layer was dried and concentrated. The crude product was purified by chromatography (EtOAc to 10% MeOH in EtOAc) to give compound 20 (387.1 mg, 0.74 mmol, 73%).

Physical state: white powder

TLC: R_{f}=0.30 (10% MeOH in EtOAc, UV active, stains brown upon KMnO₄ staining)

[α]²⁵_{D}: +49.0 (c=0.1, CHCl₃)

¹H-NMR (600 MHz, CDCl₃) δ 5.23 (s, 1H), 4.78 (br s, 1H), 4.59 (d, J=12.0 Hz, 1H), 4.25 (dt, J=11.2, 6.0 Hz, 1H), 4.09-4.02 (m, 1H), 4.00 (dd, J=11.3, 2.8 Hz, 1H), 3.95-3.86 (dt, J=13.4, 6.4 Hz, 2H), 3.45 (td, J=14.3, 7.3 Hz, 1H), 2.60-2.43 (m, 3H), 2.30 (dt, J=14.1, 11.0 Hz, 1H), 2.26-2.18 (m, 2H), 2.14-2.04 (m, 2H), 2.04-1.92 (m, 2H), 1.92-1.84 (m, 2H), 1.82 (dd, J=15.2, 7.2 Hz, 1H), 1.73 (dt, J=12.7, 7.0 Hz, 1H), 1.59 (dt, J=12.7, 6.3 Hz, 1H), 1.54 (dd, J=14.1, 2.8 Hz, 1H), 0.88 (d, J=6.9 Hz, 3H)

¹³C-NMR (150 MHz, CDCl₃) δ 204.2, 153.4, 147.8, 118.6 (q, J_{C-F}=320 Hz), 118.0, 110.7, 84.9, 78.7, 70.9, 59.5, 49.3, 40.2, 37.7, 34.3, 33.9, 33.6, 29.0, 25.6, 24.4, 22.7, 18.4, 17.6

HRMS (ESI-TOF): calc'd for: C₂₂H₂₈F₃NO₈S: [M+H]⁺: 524.1561, found: 524.1561

### Compound 21

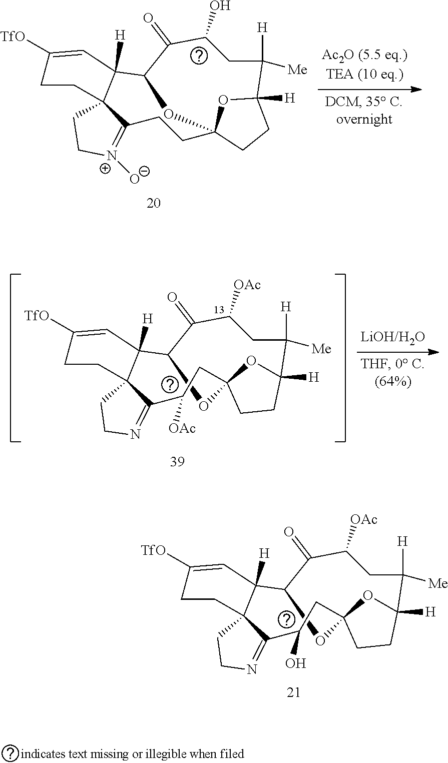

Compound 20 (132 mg, 0.25 mmol) was dissolved in DCM (0.6 ml), and TEA (0.35 ml, 253 mg, 2.50 mmol) was added. The mixture was then heated to 35° C., and Ac₂O (0.14 ml, 141 mg, 1.38 mmol) was added dropwise. The mixture was stirred overnight (monitored with TLC using 5% MeOH in DCM). Upon completion, the reaction was quenched with sat. NaHCO₃ aq. at room temperature. The mixture was extracted with DCM, and the combined organic layer was dried over MgSO₄. The solvent was removed under reduced pressure to give crude compound 39.

Physical state: white solid

TLC: R_{f}=0.42 (5% MeOH in DCM, stains brown upon KMnO₄ staining)

[α]²⁵_{D}: +5.30 (c=1.0, CHC13)

¹H-NMR (600 MHz, CDCl₃) δ 5.78 (dd, J=10.9, 8.0 Hz, 1H), 5.19 (dd, J=11.1, 2.8 Hz, 1H), 5.05 (q, J=1.7 Hz, 1H), 4.36 (d, J=12.0 Hz, 1H), 4.25 (dt, J=10.0, 6.0 Hz, 1H), 4.18-4.08 (m, 1H), 3.92 (dd, J=16.2, 9.1 Hz, 1H), 3.77 (ddd, 16.2, 10.2, 6.9 Hz, 1H), 2.64-2.41 (m, 3H), 2.41-2.26 (m, 3H), 2.23-2.13 (m, 1H), 2.12-1.95 (m, 2H), 2.08 (s, 3H), 2.05 (s, 3H), 1.93-1.73 (m, 4H), 1.60 (td, J=12.5, 7.6 Hz, 1H), 1.52 (dd, J=13.2, 2.8 Hz, 1H), 0.88 (d, J=6.5 Hz, 3H)

¹³C-NMR (150 MHz, CDCl₃) δ 199.8, 177.2, 169.3, 169.0, 148.8, 118.6 (q, J_{C-F}=320 Hz), 117.8, 108.1, 84.0, 79.9, 73.0, 72.1, 56.4, 53.7, 40.4, 39.1, 36.7, 34.0, 31.6, 29.4, 28.7, 26.5, 24.5, 21.3, 20.9, 17.4

HRMS (ESI-TOF): calc'd for C₂₆H₃₂F₃NO₁₀S: [M+H]⁺: 608.1777, found: 608.1776

The crude 39 obtained above was dissolved in THF (3.1 ml) and H₂O (0.31 ml), and a solution of LiOH (1.4 M in H₂O, 0.23 ml) was added at 0° C. The resulting solution was then stirred at 0° C., and carefully monitored by TLC (5% MeOH in DCM). The reaction was quenched by a mixture of sat. NaHCO₃ aq. and DCM, followed by extraction with DCM for three times. The organic layer was combined and dried over MgSO₄. After removal of the solvents, the crude product was purified by chromatography (pure DCM to 5% MeOH in DCM) to afford compound 21 (88.1 mg, 0.16 mmol, 64%).

Physical state: white powder

TLC: R_{f}=0.25 (5% MeOH in DCM, stains brown upon KMnO₄ staining)

[α]²⁵_{D}: +14.0 (c=0.1, CHCl₃)

¹H-NMR (600 MHz, CDCl₃) δ 5.19 (dd, J=11.4, 2.7 Hz, 1H), 5.06 (q, J=1.7 Hz, 1H), 4.75 (dd, J=11.1, 7.6 Hz, 1H), 4.34 (d, J=12.2 Hz, 1H), 4.29-4.20 (m, 2H), 3.81 (dd, J=15.7, 8.9 Hz, 1H), 3.61 (ddt, J=16.2, 10.5, 5.8 Hz, 1H), 2.72 (dd, J=14.9, 11.2 Hz, 1H), 2.57-2.25 (m, 5H), 2.19 (dd, J=15.0, 7.6 Hz, 1H), 2.04 (s, 3H), 2.03-1.98 (m, 1H), 1.98-1.93 (m, 1H), 1.91 -1.81 (m, 2H), 1.77 (dt, J=12.1, 7.1 Hz, 1H), 1.70-1.67 (m, 1H), 1.58 (dd, J=12.6, 7.7 Hz, 1H), 1.52 (dd, J=13.6, 2.8 Hz, 1H), 0.88 (d, J=6.9 Hz, 3H)

¹³C-NMR (150 MHz, CDCl₃) δ 199.6, 183.2, 169.1, 148.6, 118.7 (q, J_{C-F}=319 Hz), 118.0, 108.4, 84.0, 80.0, 72.9, 70.3, 55.4, 53.5, 42.7, 38.9, 37.0, 34.0, 31.5, 29.5, 29.1, 26.5, 24.4, 21.0, 17.5

HRMS (ESI-TOF): calc'd for C₂₄H₃₀F3NO₉S [M+H]⁺: 566.1666, found: 566.1658

### Compound 22

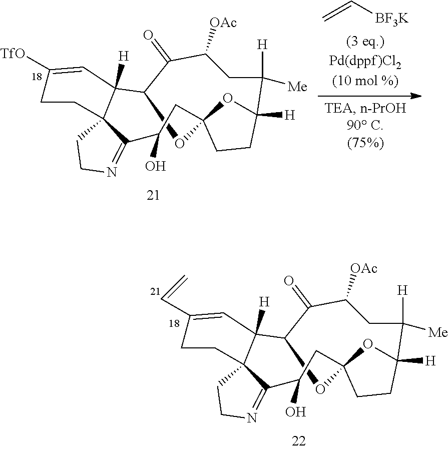

To a solution of compound 21 (39.3 mg, 0.069 mol) in n-PrOH was added vinylBF₃K (30 mg, 0.22 mol) and Pd(dppf)Cl₂ (5.1 mg, 0.007 mol) at ambient temperature. The mixture was then heated to 90° C., and TEA (0.05 ml) was added dropwise. The resulting red solution became yellow gradually, ended to a brown solution. The reaction was monitored by TLC (40% acetone in hexanes). Upon completion, the volatiles were removed by reduced pressure, and the residue was dissolved in DCM and water was added. The mixture was extracted with DCM for three times and the organic layer was dried over MgSO₄. The solvents were removed in vacuo, and the crude product was purified by chromatography (20-40% acetone in hexanes) to give compound 22 (23 mg, 0.052 mmol, 75%).

Physical state: white solid

TLC: R_{f}=0.43 (40% acetone in hexanes)

[α]²⁵_{D}: +18.0 (c=0.1, CHCl₃)

¹H-NMR (600 MHz, CDCl₃) δ 6.18 (dd, J=17.5, 10.7 Hz, 1H), 5.24-5.19 (m, 1H), 5.10 (d, J=17.5 Hz, 1H), 4.91-4.99 (m, 2H), 4.75 (dd, J=11.2, 7.4 Hz, 1H), 4.31 (d, J=12.1 Hz, 1H), 4.26 (dt, J=10.7, 5.6 Hz, 1H), 4.16 (d, J=12.1 Hz, 1H), 3.76 (dd, J=16.2, 8.8 Hz, 1H), 3.61 (ddd, J=16.2, 10.4, 6.8 Hz, 1H), 2.81 (dd, J=14.8, 11.3 Hz, 1H), 2.42-2.22 (m, 5H), 2.19-2.14 (m, 1H), 2.07 (s, 3H), 2.03 (dd, J=12.3, 7.0 Hz, 1H), 1.91-1.71 (m, 4H), 1.71-1.63 (m, 1H), 1.57 (td, J=12.5, 7.6 Hz, 1H), 1.53-1.46 (m, 1H), 0.87 (d, J=7.0 Hz, 3H)

¹³C-NMR (150 MHz, CDCl₃) δ 200.1, 184.0, 168.9, 139.0, 135.4, 127.7, 111.6, 108.2, 83.7, 80.1, 73.7, 70.8, 55.4, 54.2, 42.5, 39.8, 36.9, 34.0, 31.6, 29.6, 29.2, 24.3, 22.7, 21.1, 17.4

HRMS (ESI-TOF): calc'd for C₂₅H₃₃NO₆ [M+H]: 444.2381, found: 444.2377

Compound 2 (portimine B)

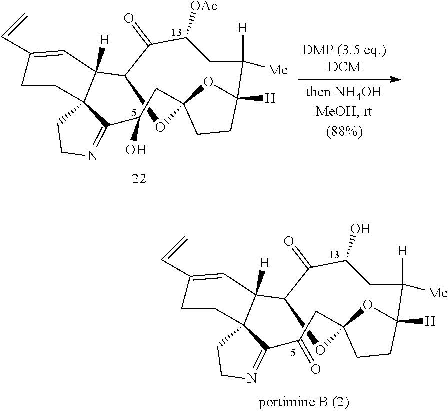

To a solution of compound 22 (16.7 mg, 0.038 mol) in DCM (0.3 ml) was added DMP (55.1 mg, 0.13 mmol) and NaHCO₃ powder (25 mg). The resulting mixture was allowed to stir at ambient temperature for 1 h. Upon completion, sat. K₂CO₃ aq. (0.2 ml) and sat. Na₂S₂O₃ aq. (0.1 ml) was added, followed by dilution with DCM. The biphasic mixture was stirred vigorously for 30 min, and the organic layer was separated. The aqueous phase was extracted with DCM, and the combined DCM phase was dried over MgSO₄ and filter. After concentration, the residue was dissolved in MeOH (0.2 ml) and added NH₄₀H (37% in H₂O, 0.1 ml). The processing of hydrolysis can be monitored by TLC (5% MeOH in DCM). After completion, the mixture was added sat. NaCl aq. to help phase separation. DCM was chosen to extract the solution (three times), and the combined DCM layer was dried and concentrated. Portimine B (2) (13.3 mg, mmol, 88%) was obtained by chromatography (pure DCM to 5% MeOH in DCM).

Physical state: white solid

TLC: R_{f}=0.22 (3% MeOH in DCM, UV active, stains brown upon KMnO₄)

[α]²⁵_{D}: +42.9 (c=0.08, CHCl₃)

¹H-NMR (600 MHz, CDCl₃) δ 6.21 (dd, J=17.4, 10.8 Hz, 1H), 5.11 (d, J=17.4 Hz, 1H), 5.07 (d, J=2.0 Hz, 1H), 4.97 (d, J=10.8 Hz, 1H), 4.62 (d, J=12.2 Hz, 1H), 4.281 (dt, J=10.1, 6.0 Hz, 1H), 4.15 (d, J=11.3 Hz, 1H), 4.08 (dd, J=16.2, 9.1 Hz, 1H), 3.83 (ddd, J=16.6, 10.4, 6.7 Hz, 1H), 3.78 (d, J=12.8 Hz, 1H), 3.43 (d, J=17.5 Hz, 1H), 2.79 (d, J=17.5 Hz, 1H), 2.50 (s, 1H), 2.42-2.24 (m, 4H), 2.12 (dd, J=12.5, 7.1 Hz, 1H), 2.03-1.90 (m, 2H), 1.87 (dd, J=12.5, 6.6 Hz, 1H), 1.78 (dt, J=12.4, 6.4 Hz, 2H), 1.73-1.65 (m, 1H), 1.63-1.53 (m, 1H), 0.91 (d, J=7.1 Hz, 3H)

¹³C-NMR (150 MHz, CDCl₃) δ 204.4, 200.9, 182.5, 138.6, 136.0, 126.7, 112.4, 107.1, 84.7, 78.9, 72.2, 58.1, 54.7, 53.0, 40.9, 38.2, 34.3, 34.1, 28.7, 28.6, 24.4, 22.6, 17.7

HRMS (ESI-TOF): calc'd for C₂₃H₂₉NO₅ [M+H]f: 400.2119, found: 400.2117

TABLE 2
Summary of ¹³C shift between synthetic portimine B (2) and
authentic portimine B (1) in CDCl₃

| position | chemical shift in authentic | chemical shift in synthetic |
|---|---|---|
| C1 | 58.1 | 58.4 |
| C2 | 28.7 | 28.8 |
| C3 | 54.7 | 55.1 |
| C4 | 182.5 | 182.9 |
| C5 | 200.9 | 201.4 |
| C6 | 53.0 | 53.2 |
| C7 | 107.1 | 107.2 |
| C8 | 38.2 | 38.5 |
| C9 | 24.4 | 24.6 |
| C10 | 84.7 | 85.0 |
| C11 | 34.3 | 34.7 |
| C12 | 34.1 | 34.3 |
| C13 | 78.9 | 79.1 |
| C14 | 204.4 | 204.8 |
| C15 | 72.2 | 72.4 |
| C16 | 40.9 | 41.2 |
| C17 | 126.7 | 126.9 |
| C18 | 136.0 | 135.8 |
| C19 | 22.6 | 22.8 |
| C20 | 28.6 | 28.8 |
| C21 | 138.6 | 138.8 |
| C22 | 112.4 | 112.5 |
| C23 | 17.7 | 18.0 |

Compound 1 (portimine A)

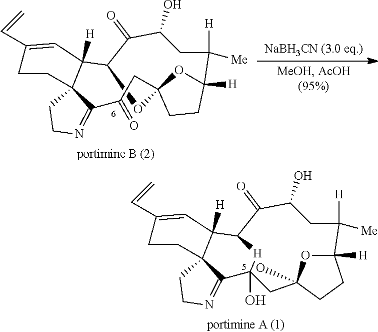

To a solution of crude compound 2 (ca. 20 mg, 0.05 mmol) in MeOH (0.5 ml) was added NaBH3CN (10 mg, 0.16 mol) and AcOH (0.025 ml) at ambient temperature. The resulting mixture was stirred at the same temperature for 1.5 h, and quenched with sat. NaHCO₃ aq. The mixture was extracted with DCM for four times, and the organic layer was dried over MgSO₄ and concentrated. The crude product was purified with chromatography (DCM to 10% MeOH in DCM) to obtain compound 1 (19.2 mg, 0.048 mol, 95%) Physical state: white solid

TLC: R_{f}=0.54 (10% MeOH in DCM, UV active, stains brown upon KMnO₄ staining)

[α]²⁵_{D}: +5.8 (c=1.0, CHCl₃)

¹H-NMR (600 MHz, CDCl₃) δ 6.21 (dd, J=17.6, 10.8 Hz, 1H), 5.17-5.01 (m, 2H), 4.93 (d, J=10.8 Hz, 1H), 4.71 (br s, 1H), 4.52 (s, 1H), 4.47 (d, J=12.0 Hz, 1H), 4.18 (dt, J=11.2, 5.6 Hz, 1H), 4.08 (dd, J=11.2, 2.7 Hz, 1H), 3.86 (dd, J=15.2, 8.8 Hz, 1H), 2.81 (dd, J=15.9, 5.1 Hz, 1H), 2.43-2.19 (m, 4H), 2.09 (d, J=15.8 Hz, 1H), 2.01-1.92 (m, 2H), 1.93-1.79 (m, 2H), 1.79-1.61 (m, 5H), 1.50 (dd, J=13.7, 2.6 Hz, 1H), 0.88 (d, J=6.9 Hz, 3H)

¹³C-NMR (150 MHz, CDCl₃) δ 204.6, 185.0, 138.8, 135.2, 127.8, 111.7, 109.1, 83.2, 79.3, 71.6, 65.6, 54.9, 52.7, 43.1, 40.6, 37.6, 34.3, 33.8, 29.6, 29.0, 24.6, 22.6, 17.6

HRMS (ESI-TOF): calc'd for C₂₃H₃₁NO₅ [M+H]⁺: 402.2275, found: 402.2265

TABLE 2
Summary of ¹³C shift between synthetic portimine A (1) and
authentic portimine A (1) in CDCl₃

| position | chemical shift in authentic | chemical shift in synthetic |
|---|---|---|
| C1 | 54.9 | 54.9 |
| C2 | 29.4 | 29.6 |
| C3 | 52.7 | 52.7 |
| C4 | 185.0 | 185.0 |
| C5 | 65.6 | 65.6 |
| C6 | 43.1 | 43.1 |
| C7 | 109.1 | 109.1 |
| C8 | 37.6 | 37.6 |
| C9 | 24.6 | 24.6 |
| C10 | 83.2 | 83.2 |
| C11 | 34.3 | 34.3 |
| C12 | 33.8 | 33.8 |
| C13 | 79.3 | 79.3 |
| C14 | 204.6 | 204.6 |
| C15 | 71.6 | 71.6 |
| C16 | 40.7 | 40.6 |
| C17 | 127.8 | 127.8 |
| C18 | 135.2 | 135.2 |
| C19 | 22.6 | 22.6 |
| C20 | 29.1 | 29.0 |
| C21 | 138.8 | 138.8 |
| C22 | 111.7 | 111.7 |
| C23 | 17.6 | 17.6 |

Compund 33-1

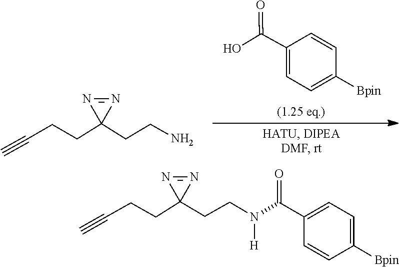

To a solution of amine (16 mg, 0.12 mmol) in DMF (0.2 ml) was added HATU (44 mg, 0.12 mmol) and acid (38 mg, 0.15 mmol). The mixture was stirred at rt, followed by an addition of DIPEA (10 drops). The resulting yellow solution was stirred under argon for 16 h, and diluted with Et20. Sat. NaHCO₃ aq. was added to this mixture to quench the excess benzoic acid. The resulting biphasic mixture was extracted with Et20, dried over MgSO₄, and concentrated under reduced pressure. Pure compound 33-1 (32 mg, 0.087 mmol, 73%) was obtained by PTLC (12% Et20 in DCM).

Physical state: colorless oil

TLC: R_{f}=0.55 (30% EtOAc in hexanes, UV active, stains brown upon KMnO₄ staining)

¹H-NMR (600 MHz, CDCl₃) δ 7.87 (d, J=8.2 Hz, 2H), 7.77 (d, J=8.2 Hz, 2H), 6.35 (t, J=5.8 Hz, 1H), 3.31 (q, J=6.6 Hz, 2H), 2.03 (td, J=7.2, 2.6 Hz, 2H), 1.98 (t, J=2.6 Hz, 1H), 1.82 (t, J=6.6 Hz, 2H), 1.68 (t, J=7.2 Hz, 2H), 1.35 (s, 12H)

¹³C-NMR (150 MHz, CDCl₃) δ 167.7, 136.7, 135.2, 126.2, 84.3, 82.9, 69.6, 35.1, 32.7, 32.3, 29.9, 27.1, 25.0, 13.4

HRMS (ESI-TOF): calc'd for C₂₀H₂₆BN₃O₃ [M+H]⁺: 368.2140, found: 368.2136

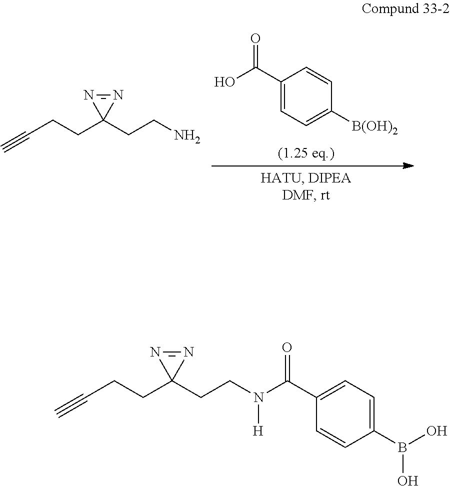

To a solution of amine (10.0 mg, 0.12 mmol) in DMF (0.2 ml) was added HATU (36 mg, 0.12 mmol) and acid (16 mg, 0.15 mmol). The mixture was stirred at rt, followed by an addition of DIPEA (0.05 ml). The resulting yellow solution was stirred under argon for 16 h, followed by adding H₂O. The resulting biphasic mixture was extracted with EtOAc, dried over MgSO₄, and concentrated under reduced pressure. Pure compound 33-2 (17.2 mg, 0.087 mmol, 73%) was obtained by PTLC (60% EtOAc in hexanes).

Physical state: white solid

TLC: R_{f}=0.65 (60% EtOAc in hexanes, UV active, stains brown upon KMnO₄ staining)

¹H-NMR (500 MHz, CD₃OD) δ 7.79 (d, J=7.6 Hz, 2H), 7.70 (d, J=7.8 Hz, 2H), 2.27 (t, J=2.7 Hz, 1H), 2.05 (td, J=7.4, 2.7 Hz, 2H), 1.75 (t, J=7.1 Hz, 2H), 1.68 (t, J=7.4 Hz, 2H)

¹³C-NMR (126 MHz, CD₃OD) δ 169.0, 133.5, 125.8, 82.2, 69.0, 37.5, 34.6, 32.1, 31.8, 26.6, 12.5

HRMS (ESI-TOF): calc'd for C14H16BN3O3 [M+H]⁺: found: 286.1354

### Compound 34

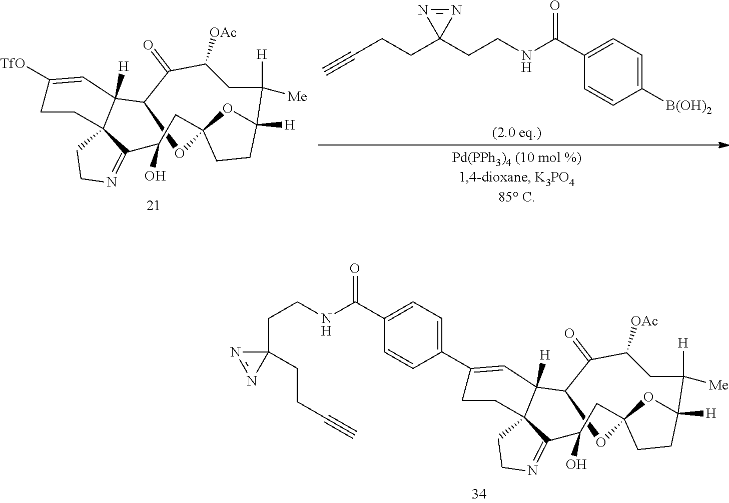

To a solution of compound 21 (5.0 mg, 0.0088 mmol) and compound 33-2 (5.1 mg, 0.018 mmol) in 1,4-dioxane (0.35 ml) was added Pd(PPh₃)₄ (0.7 mg, 0.00091 mmol) and K₃PO₄ (3 mg). The resulting yellow solution was heated to 85° C. Upon completion (normally 2 h), the mixture was added H₂O and extracted with DCM. The pure compound 34 (4.5 mg, 0.0069 mmol, 78%) was obtained by PTLC (45% acetone in hexanes).

Physical state: white solid

TLC: R_{f}=0.25 (10% MeOH in DCM, UV active, stains brown upon KMnO₄ staining)

¹H-NMR (600 MHz, CDCl₃) δ 7.70 (d, J=8.5 Hz, 2H), 7.36 (d, J=8.5 Hz, 2H), 6.29 (t, J=5.9 Hz, 1H), 5.41 (s, 1H), 5.25-5.20 (m, 1H), 4.80 (dd, J=11.1, 7.5 Hz, 1H), 4.39 (d, J=12.1 Hz, 1H), 4.29-4.20 (m, 2H), 3.82 (dd, J=15.6, 8.6 Hz, 1H), 3.68 (ddd, J=16.0, 10.1, 7.0 Hz, 1H), 3.31 (q, J=6.4 Hz, 2H), 2.84 (dd, J=14.8, 11.2 Hz, 1H), 2.64-2.52 (m, 2H), 2.47-2.32 (m, 3H), 2.21 (dd, J=14.9, 7.5 Hz, 1H), 2.04 (s, 3H), 2.04-2.02 (m, 2H), 1.98 (t, J=2.6 Hz, 2H), 1.98-1.84 (m, 3H), 1.82 (t, J=6.6 Hz, 2H), 1.81-1.74 (m, 3H), 1.68 (t, J=7.3 Hz, 2H), 1.59 (td, J=12.6, 7.7 Hz, 1H), 1.53 (dd, J=13.1, 2.9 Hz, 1H), 0.89 (d, J=6.2 Hz, 3H)

¹³C-NMR (150 MHz, CDCl₃) δ 200.3, 184.5, 169.1, 167.3, 144.1, 135.6, 133.1, 127.2, 125.2, 124.9, 108.4, 83.9, 82.9, 80.3, 73.8, 70.8, 69.7, 55.4, 53.8, 42.7, 40.1, 37.0, 35.1, 34.2, 32.7, 32.3 (d, J=5.55 Hz), 31.7, 30.1, 29.9, 29.3, 27.1, 26.0, 24.5, 21.3, 17.6

HRMS (ESI-TOF): calc'd for C₃₇H₄₄N₄O₇ [M+H]⁺: 657.3283, found: 657.3291

### Compound 35-1

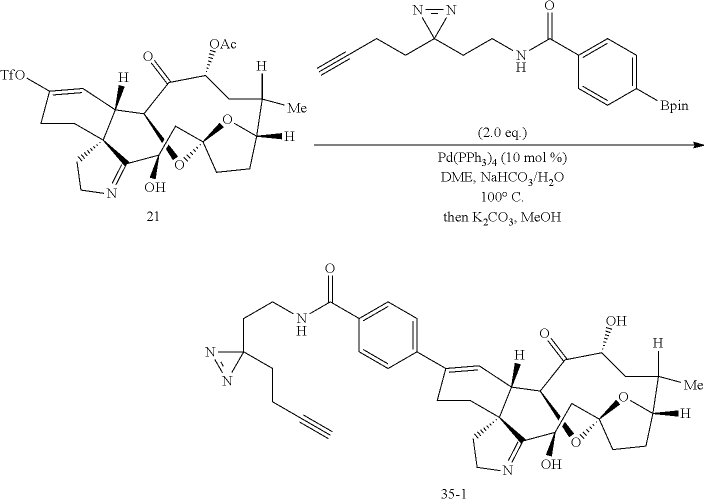

To a solution of compound 21 (5.1 mg, 0.0091 mmol) and compound 33 (6.6 mg, 0.018 mmol) in DME (0.2 ml) was added Pd(PPh₃)₄ (1.0 mg, 0.00091 mmol). The resulting yellow solution was added sat. NaHCO₃ aq. (10 drops) while stirring, followed by heating to 100° C. Upon completion (normally 2 h), the mixture was added H₂O and extracted with DCM. The combined organic layer was dried over MgSO₄ and concentrated. The residue was dissolved in MeOH (0.1 ml) and treated with K₂CO₃ (10 mg). After vigorous stirring at room temperature for 30 min, the mixture was extracted with DCM and purified by PTLC (8% MeOH in DCM) to afford compound 35 (4.4 mg, 0.0072 mmol, 79%) as product.

Physical state: white solid

TLC: R_{f}=0.21 (10% MeOH in DCM, UV active, stains brown upon KMnO₄ staining)

¹H-NMR (600 MHz, CDCl₃) δ 7.78 (d, J=8.0 Hz, 2H), 7.46 (d, J=8.0 Hz, 2H), 7.12 (br s, 1H), 5.63 (s, 1H), 4.74 (t, J=9.3 Hz, 1H), 4.62 (d, J=11.9 Hz, 1H), 4.31 (s, 1H), 4.14-4.03 (m, 1H), 3.92 (d, J=12.0 Hz, 1H), 3.59 (q, J=6.5 Hz, 1H), 3.54-3.45 (m, 1H), 3.42-3.30 (m, 1H), 2.96 (q, J=9.1 Hz, 1H), 2.85 (t, J=12.9 Hz, 1H), 2.43-2.29 (m, 2H), 2.29-2.20 (m, 2H), 2.18-2.09 (m, 1H), 2.05-1.87 (m, 6H), 1.86-1.70 (m, 4H), 1.68 (td, J=7.2, 4.5 Hz, 2H), 1.65-1.55 (m, 3H), 1.54-1.44 (m, 1H), 0.95 (d, J=6.5 Hz, 3H)

¹³C-NMR (150 MHz, CDCl₃) δ 207.8, 185.1, 167.7, 142.3, 133.8, 132.9, 127.4, 124.7, 124.4, 108.1, 84.3, 82.9, 79.6, 71.5, 70.8, 69.6, 54.7, 54.0, 53.8, 42.4, 40.3, 37.1, 34.9, 34.6, 33.0, 32.7, 32.3, 29.8, 29.4, 28.9, 27.2, 25.5, 25.1, 24.5, 17.8

HRMS (ESI-TOF): calc'd for C₃₅H₄₂N₄O₆ [M+H]⁺: 615.3177, found: 615.3170

### Compound 35-2

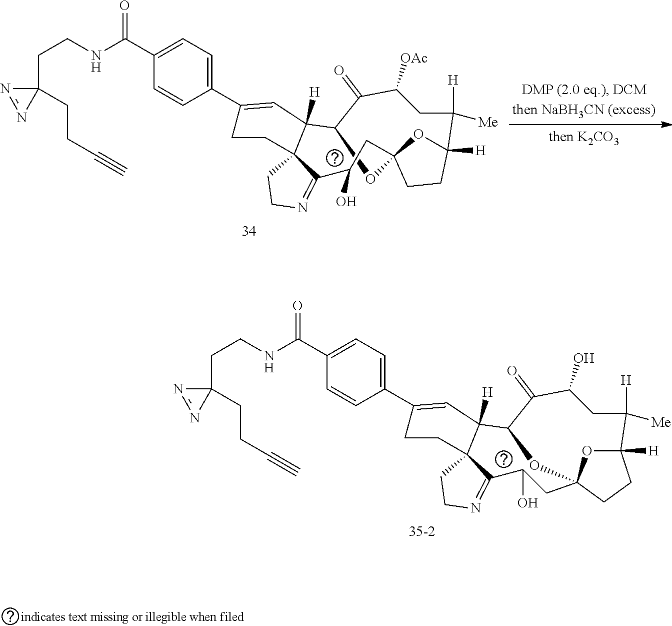

To a solution of compound 34 (4.5 mg, 0.0069 mmol) in DCM (0.8 ml) was added DMP (5.9 mg, 0.014 mmol) at room temperature (ca. 20° C.) and NaHCO₃ (10 mg). The mixture was then stirred for 2 h, followed by quenching with sat. Na₂S₂O₃ aq. and solid Na₂CO₃. The mixture was then stirred for 30 min to give a clear biphasic liquid. The DCM layer was collected and the aqueous layer was extracted with DCM for 3 times. The organic phase was dried over MgSO₄ and concentrated to give the crude product, which was dissolved in MeOH (0.15 ml) and treated with AcOH (5 drops). The solution was cooled to 0° C., and NaBH3CN (ca. 5 mg, large excess) was added in one portion. The colorless solution was stirred at the same temperature for 1.5 h. Upon completion, K₂CO₃ (100 mg) and H₂O (0.1 ml) was added subsequently, which gave a slightly yellow mixture. The hydrolysis was carried out at room temperature for 1 h, and the mixture was extracted with DCM for 4 times. The organic layer was dried over MgSO₄ and concentrated. Pure compound 35-2 (2.1 mg, 0.0034, 49%) was obtained by PTLC (10% MeOH in DCM).

Physical state: white solid

TLC: R_{f}=0.23 (10% MeOH in DCM, UV active, stains brown upon KMnO₄ staining)

¹H-NMR (600 MHz, CDCl₃) δ 7.69 (d, J=8.1 Hz, 2H), 7.40 (d, J=8.2 Hz, 2H), 6.31 (s, 1H), 5.54 (d, J=2.2 Hz, 1H), 4.56 (d, J=12.0 Hz, 1H), 4.43 (s, 1H), 4.21 (dt, J=11.2, 5.9 Hz, 1H), 4.10 (dd, J=11.3, 2.7 Hz, 1H), 3.90-3.83 (m, 1H), 3.72-3.62 (m, 2H), 3.30 (q, J=6.4 Hz, 2H), 2.80 (dd, J=15.9, 5.1 Hz, 1H), 2.54 (d, J=7.8 Hz, 2H), 2.39-2.32 (m, 1H), 2.31-2.25 (m, 1H), 2.13 (d, J=15.9 Hz, 1H), 2.07-1.96 (m, 5H), 1.92 (dd, J=12.6, 6.7 Hz, 1H), 1.89-1.83 (m, 1H), 1.81 (t, J=6.6 Hz, 2H), 1.75 (td, J=12.3, 5.1 Hz, 2H), 1.72-1.63 (m, 3H), 1.61-1.51 (m, 2H), 0.90 (d, J=6.9 Hz, 3H)

¹³C-NMR (150 MHz, CDCl₃) δ 205.5, 185.2, 167.3, 143.7, 135.0, 133.0, 127.1, 125.4, 109.2, 83.5, 82.9, 79.4, 71.6, 69.7, 65.7, 55.0, 52.1, 43.2, 40.8, 37.8, 35.1, 34.5, 33.8, 32.6, 32.3, 29.9, 29.5, 29.3, 27.2, 25.7, 24.8, 17.8

HRMS (ESI-TOF): calc'd for C₃₅H₄₂N₄O₆ [M+H]⁺: 615.3177, found: 615.3193

### Compound 36 (Ref. 7)

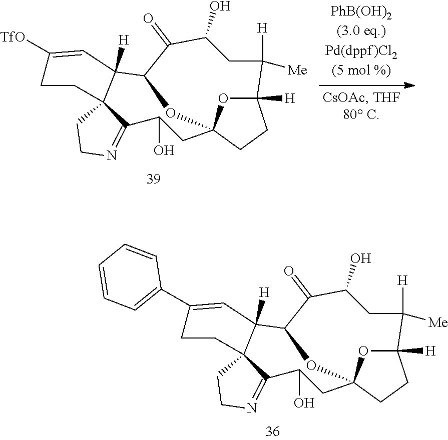

To a mixture of compound 39 (2.3 mg, 0.0044 mmol), PhB(OH)₂ (1.6 mg, 0.013 mmol), CsOAc (5 mg), and Pd(dppf)Cl₂—CH₂Cl₂ (0.18 mg, 0.0002 mmol) was added THE (0.3 ml) and heated to reflux. After 20 h, TLC indicated the full completion (monitored by 40% acetone in hexanes), and a mixture of brine and DCM was added. The mixture was extracted with DCM for 4 times, dried over MgSO₄, and concentrated. Pure compound 36 (1.7 mg, 0.0037 mmol, 84%) was obtained by PTLC (10% MeOH in DCM).

Physical state: white powder

TLC: R_{f}=0.46 (10% MeOH in DCM, stains brown upon KMnO₄ staining)

¹H-NMR (600 MHz, CDCl₃) δ 7.39 (d, J=7.4 Hz, 2H), 7.29 (t, J=7.4 Hz, 2H), 7.24 (t, J=7.4 Hz, 1H), 5.47 (s, 1H), 4.53 (d, J=12.1 Hz, 1H), 4.37 (s, 1H₂), 4.21 (dt, J=11.2, 6.0 Hz, 1H), 4.09 (dd, J=11.2, 2.6 Hz, 1H), 3.84 (dd, J=15.3, 9.0 Hz, 1H), 3.71-3.61 (m, 1H), 3.55 (d, J=12.1 Hz, 1H), 2.78 (dd, J=15.9, 5.1 Hz, 1H), 2.58-2.41 (m, 2H), 2.39-2.24 (m, 2H), 2.11 (d, J=15.9 Hz, 1H), 2.01-1.93 (m, 2H), 1.93-1.81 (m, 2H), 1.76-1.70 (m, 1H), 1.71-1.63 (m, 3H), 1.55 (dd, 0.1=14.1, 2.7 Hz, 1H), 0.91 (d, 0.1=6.9 Hz, 3H)

¹³C-NMR (150 MHz, CDCl₃) δ 206.1, 140.4, 135.4, 128.5, 127.6, 125.2, 123.4, 109.2, 83.4, 79.6, 71.7, 65.7, 54.8, 52.1, 43.1, 40.5, 37.7, 34.5, 33.5, 29.9, 29.4, 29.1, 25.6, 24.8, 17.8

(1 carbon (C=N) was missing in ¹³C NMR)

HRMS (ESI-TOF): calc'd for C₂₇H₃₃NO₅ [M+H]⁺: 452.2432, found: 452.2435

### Compound 37

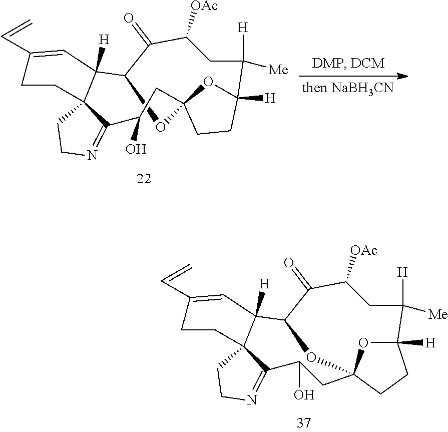

Compound 37 (6.3 mg, 0.014 mmol) was prepared from compound 22 (9.0 mg, 0.020 mmol) by oxidation with DMP and reduction with NaBH3CN. Purified by PTLC (5% i-PrOH in DCM).

Physical state: white powder

TLC: R_{f}=0.58 (10% MeOH in DCM, UV active, stains brown upon KMnO₄ staining)

¹H-NMR (600 MHz, CDCl₃) δ 6.16 (dd, J=17.5, 10.8 Hz, 1H), 5.21-5.16 (m, 1H), 5.10 (d, J=17.5 Hz, 1H), 4.96 (d, J=10.8 Hz, 1H), 4.90 (s, 1H), 4.52 (t, J=4.2 Hz, 1H), 4.29 (d, J=11.9 Hz, 1H), 4.20 (dt, J=10.8, 5.4 Hz, 1H), 3.88 (dd, J=15.3, 8.9 Hz, 1H), 3.75 (d, J=12.4 Hz, 1H), 3.70 (ddd, J=14.7, 7.1, 3.7 Hz, 1H), 2.81 (dd, J=16.0, 5.1 Hz, 1H), 2.45-2.26 (m, 4H), 2.11 (dd, J=16.1, 1.9 Hz, 1H), 2.06 (s, 3H), 1.99 (dd, J=12.2, 7.1 Hz, 2H), 1.93 (dd, J=12.5, 6.9 Hz, 1H), 1.88-1.63 (m, 5H), 1.49 (dd, J=12.9, 2.8 Hz, 1H), 0.87 (d, J=6.6 Hz, 3H)

¹³C-NMR (150 MHz, CDCl₃) δ 200.5, 185.8, 169.0, 138.8, 135.6, 127.3, 112.2, 109.4, 83.3, 80.2, 73.6, 65.7, 54.8 (d, J=2.8 Hz), 52.8, 43.3, 40.7, 37.8, 34.1, 31.6, 29.5, 29.0, 24.7, 22.7, 21.3, 17.6

HRMS (ESI-TOF): calc'd for C₂₅H₃₃NO₆ [M+H]⁺: 444.2381, found: 444.2379

### Compound 38

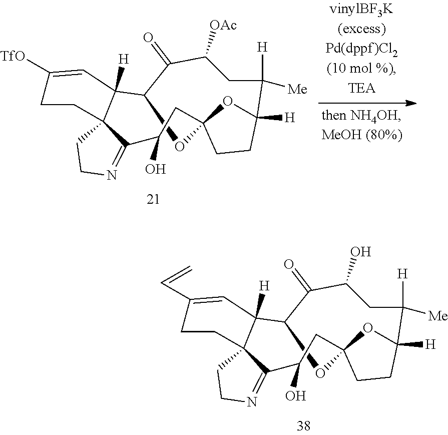

Compound 21 (2.2 mg, 0.005 mmol) was dissolved in n-PrOH (0.1 ml), followed by addition of vinylBF₃K (1.5 mg, 0.011 mmol) and Pd(dppf)Cl₂ (0.4 mg, 0.0005 mmol) under argon. TEA (1 drop) was added to this solution. Subsequently, the reaction was heated to 80° C. for 1 h. Upon completion (monitored by TLC), the solvent was removed under vacuo, and the residue was dissolved in MeOH (0.1 ml). Ammonia hydroxide (aqueous solution, 37%, 0.1 ml) was added to this solution. The resulting solution was stirred for 2 h, and concentrated. The residue was purified by PTLC (10% i-PrOH in DCM) to give compound 38 (epi-portimine A) (1.8 mg, 0.004 mmol, ca. 80%) as single product.

Physical state: white solid

TLC: R_{f}=0.44 (10% MeOH in DCM, UV active, stains brown upon KMnO₄ staining)

[α]²⁵_{D}: −8.0 (c=0.1, CHC13)

¹H-NMR (600 MHz, CDCl₃) δ 6.21 (dd, J=17.5, 10.8 Hz, 1H), 5.08 (d, J=17.5 Hz, 1H), 5.04 (s, 1H), 4.93 (d, J=10.8 Hz, 1H), 4.78 (dd, J=11.1, 7.5 Hz, 1H), 4.47 (d, J=12.0 Hz, 1H), 4.23 (dt, J=10.4, 6.0 Hz, 1H), 4.14 (d, J=12.0 Hz, 1H), 4.08 (dd, J=11.2, 2.7 Hz, 1H), 3.75 (dd, J=16.0, 8.8 Hz, 1H), 3.63 (ddd, J=16.0, 10.3, 7.0 Hz, 1H), 2.80 (dd, J=14.6, 11.1 Hz, 1H), 2.40-2.21 (m, 5H), 2.15 (dd, J=14.0, 6.7 Hz, 1H), 2.01 (dd, J=12.1, 7.1 Hz, 1H), 1.92-1.82 (m, 2H), 1.79 (dd, J=12.5, 6.7 Hz, 1H), 1.76-1.66 (m, 2H), 1.58-1.49 (m, 2H), 0.88 (d, J=6.0 Hz, 3H)

¹³C-NMR (150 MHz, CDCl₃) δ 204.7, 184.8, 139.1, 135.3, 128.2, 111.6, 108.2, 83.9, 79.3, 71.9, 70.7, 55.3, 54.5, 42.5, 40.1, 37.0, 34.6, 33.9, 29.8, 29.2, 24.5, 22.8, 17.7

HRMS (ESI-TOF): calc'd for C₂₃H₃₁NO₅ [M+H]⁺: 402.2275, found: 402.2266

### Compound 39 and 40

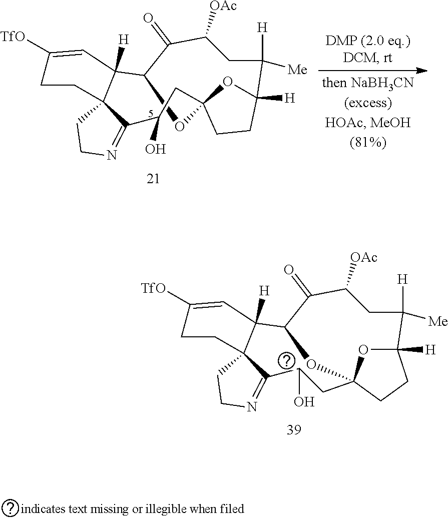

Compound 21 (15.4 mg, 0.027 mmol) was dissolved in DCM (0.15 ml), followed by addition of Dess-Martin periodinane (27.2 mg, mmol). After stirring under ambient temperature for 1.5 h, the mixture was added DCM and sat. Na₂S₂O₃ aq., and solid Na₂CO₃. The heterogenous, cloudy mixture was stirred at the same temperature for 30 min to give a clear biphasic mixture. The DCM layer was collected, and the aqueous phase was extracted with DCM for 3 times. After concentration, the crude product was dissolved in MeOH (0.3 ml), followed by adding AcOH (0.05 ml) at room temperature. NaBH3CN (ca. 10 mg) was added to this solution in one portion, and the resulting clear solution was stirred at the same temperature for 1 h. Upon completion (indicated by TLC, 5% MeOH in DCM), K₂CO₃ (55 mg) was added in one portion to give a slightly yellow mixture. The mixture was stirred for 30 min, then quenched by adding H₂O. The mixture was extracted with DCM for 3 times, and dried over MgSO₄. Pure compound 39 (11.1 mg, 0.021 mmol, 78%) was obtained by PTLC (6% i-PrOH in DCM).

Physical state: white solid

TLC: R_{f}=0.53 (10% MeOH in DCM, stains brown upon KMnO₄ staining)

[α]²⁵_{D}: (c=0.1, CHCl₃)

¹H-NMR (600 MHz, CDCl₃) δ 5.18 (dd, J=11.3, 2.8 Hz, 1H), 5.04 (s, 1H), 4.66 (br s, 1H), 4.48 (s, 1H), 4.34 (d, J=12.0 Hz, 1H), 4.21 (dt, J=11.1, 5.9 Hz, 1H), 3.94 (dd, J=15.5, 9.0 Hz, 1H), 3.86 (d, J=12.3 Hz, 1H), 3.79-3.70 (m, 1H), 2.74 (dd, J=16.2, 5.1 Hz, 1H), 2.60-2.46 (m, 2H), 2.38-2.25 (m, 2H), 2.18-2.05 (m, 3H), 2.04 (s, 3H), 2.01 (dt, J=12.4, 6.3 Hz, 1H), 1.88-1.78 (m, 3H), 1.78-1.66 (m, 2H), 1.52 (dd, J=13.6, 2.9 Hz, 1H), 0.88 (d, J=6.7 Hz, 3H)

¹³C-NMR (150 MHz, CDCl₃) δ 200.0, 169.1, 148.5, 117.9, 109.5, 83.6, 80.0, 72.8, 65.6, 55.0, 52.0, 43.4, 39.6, 37.8, 34.0, 31.5, 29.6, 28.8, 26.4, 24.7, 21.0, 17.6

(2 carbons (C═N and CF₃) were missing in ¹³C NMR but shown in compound 39)

HRMS (ESI-TOF): calc'd for C₂₄H₃₀F₃NO₉S [M+H]⁺: 566.1666, found: 566.1641

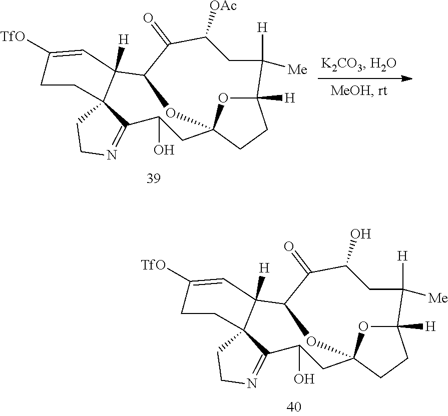

Compound 40 (11.1 mg, mmol) was obtained by treating 39 (15.5 mg, mmol) with MeOH (1.0 ml) and K₂CO₃ (58 mg).

Physical state: white solid

TLC: R_{f}=0.50 (10% MeOH in DCM, stains brown upon KMnO₄ staining)

[α]²⁵_{D}: −15.0 (c=0.1, CHC13)

¹H-NMR (600 MHz, CDCl₃) δ 5.16 (s, 1H), 4.52 (d, J=12.0 Hz, 1H), 4.48 (s, 1H), 4.19 (dt, J=11.1, 5.8 Hz, 1H), 4.08-4.00 (m, 1H), 3.92 (dd, J=15.5, 9.0 Hz, 1H), 3.81 (d, J=12.0 Hz, 1H), 3.78-3.68 (m, 1H), 2.71 (dd, J=16.1, 5.1 Hz, 1H), 2.63-2.52 (m, 1H), 2.50-2.42 (m, 1H), 2.32-2.20 (m, 2H), 2.15-2.04 (m, 2H), 2.04-1.93 (m, 2H), 1.84 (dq, J=13.1, 8.9, 6.2 Hz, 3H), 1.73 (dt, J=12.2, 6.9 Hz, 1H), 1.66 (td, J=12.7, 7.6 Hz, 1H), 1.52 (dd, J=13.2, 2.7 Hz, 1H), 0.89 (d, J=6.5 Hz, 3H)

¹³C-NMR (150 MHz, CDCl₃) δ 204.8, 148.3, 118.7, 118.6 (q, J_{C-F}=320 Hz), 109.3, 83.6, 79.0, 70.6, 65.6, 54.8, 52.0, 43.2, 39.7, 37.7, 34.2, 33.4, 29.9, 29.4, 28.7, 26.2, 24.7, 17.7

HRMS (ESI-TOF): calc'd for C₂₂H₂₈F₃NO₈S [M+H]⁺: 524.1561, found: 524.1547

### REFERENCES

1. Synthesis of Medium-Sized Cyclic Amines by Selective Ring Cleavage of Sulfonylated Bicyclic Amines. Iradier, F.; Gomez Arrayas, R.; Carretero, J. C. Org. Lett. 2001, 3, 2957-2960.
2. Microwave-Assisted Palladium-Catalyzed Allylation of β-Enaminones. Erray, I.; Rezgui, F.; Oble, J.; Poli, G. Synlett 2014, 25, 2196-2200.
3. Activation of Mg Metal for Safe Formation of Grignard Reagents on Plant Scale. Organic Process Research & Development 2002, 6, 906-910.

### Biological Assays

1) Portimine A and its functional analogs show stereo-selective acute toxicity in cancer cells (FIG. 1). (A) PA and Ph-PA have similar cell toxicity in breast (HCC1806) and leukemia (Jurkat) cancer cell lines after 36-hour treatment while PB and ePA show minimal effects. (B) Structure of portimine related analogs: phenyl-portimine A (Ph-PA, 36) and epi-portimine A (ePA, 38). Structure of fully functionalized portimine A-based epimeric probe pair: portimine A-diazirine-alkyne (PA-DA, 35-2) and epi-portimine A-diazirine-alkyne (ePA-DA, 35-1). (C) Cell viability analysis of portimine A and analogs. Jurkat cells were treated with increasing concentrations of portimine A and analogs for 24 hours. All presented data as mean of biological replicated experiments (n=3). (D) Immunoblot of caspase 3 shows PA, PA-DA, Ph-PA induce cell apoptosis, while ePA, ePA-DA and PB do not. Jurkat cells were treated with portimine A or analogs for 12 hours at 10 nM. PA and Ph-PA have ~2 nM IC50 to Jurkat cells after 12 hr. PA and Ph-PA substantially less toxic to healthy PBMCs compared to cancer T cell line Jurkat. Structurally related negative control ePA and PB are non-toxic in all cell types in the current concentrate range. PBMCs (25k per well), Jurkat (10k per well). Compound concentration was increased from 0.23 pM to 1 uM, 4× per gradient, PBMCs using primary B cell media from Wesley (heat deactivated), and Jurkat using normal RPMI media and incubated for 12 hr.
2) Portimine A triggers cell cycle arrest in Jurkat cells. Cell cycle analysis in Jurkat cells (FIG. 2). Jurkat cells were treated with 10 nM of indicated compounds for 24 hours, and propodium iodide was used to identify different stages of cells by flow cytometry. Treatment condition: Jurkat (50 k per well); Portimine A concentration: 0.007 nM-480 nM; Washout time point: 0.5, 2, 4, 6, 12, 24 hour, cells were washed once and suspended in PA-free RPMI media; In total treat cell for 36 hours; Measure cell viability with Cell Titerglo assay; Observe exposure-dependent decrease in IC50; Removing compound after 30 min results in ~5-fold decrease in IC50; IC50 after 30 min exposure ~5 nM; 4 hr ~2 nM; Overall, suggests fast acting cytotoxicity mechanism.
3) Portimine A and analogs do not induce apoptosis or affect cell viability in freshly isolated human PBMCs (FIG. 3). (A) PA and Ph-PA show less toxic to healthy PBMCs compared to Jurkat cells. Both ePA and PB showed non-toxicity to both cells in the pM-nM concentration range. (B) Immunoblot of caspase-3 in Jurkat and PBMCs with indicated conditions. Both PA and PA-DA induced caspase-3 cleavage in a dose-dependent manner in Jurkat cells, but not human PBMCs. PTMA-DA found to have analogous activity to PTMA 4 Suitable target ID probe; epi-PTMA-DA and epi-PTMA-Ac-DA has no activity in the concentrate range, same as epi-PTMA 4 useful inactive probe.
4) Portimine A mouse pharmacokinetic properties and fast acting in vitro cell-based target engagement properties based on compound wash-out (FIG. 4). (A) Pharmacokinetic studies of mouse intraperitoneal (i.p.) and oral (p.o.) administration for portimine (n=3). (B) Washout experiment performed in Jurkat and MC38 cells showed exposure-dependent decrease in IC₅₀, reveals PA has a fast-acting cytotoxicity mechanism. Impact of PA [0.3 or 1 mg/kg intraperitoneally] on MC38 tumor growth in WT mice (n=6). (C) Kaplan-Myer survival curve of WT MC38 tumor-bearing mice (n=6) after treatment with PA as described in (D). Mice were euthanized when tumor area exceeded 2000 mm³. Statistical analysis was performed using ANOVA analysis followed by multiple comparisons test, * p≤0.05. Treatment condition: Jurkat (60 M per condition); PA-DA/ePA-DA concentration: 0.5 uM, PA or ePA: 5 uM; Incubate with probe with or without competitors (PA, ePA) for 30 min; UV irradiated for 20 min. (0.5 uM PA-DA add a no-UV condition), Standard pulldown & biotin-enrichment and western blot analysis (Elute condition: 2× sample buffer+500 uM biotin, boil for 20 mins); Primary antibody NMD3 (ProteinTech, 1:150), Actin (BioRad, 1:10000). Conclusion: NMD3 can be selectively binds by Portimine A; The interaction between NMD3 and Portimine A is non-covalent; Note -PA-DA binding to recombinantly overexpressed NMD3 in cells not observed, only to endogenous NMD3. Could suggest PA binds NMD3 only when in an endogenous complex.
5) Chemical proteomic analysis reveals NMD3 is the target of portimine A (FIG. 5). Chemical proteomic profiling with PA-DA in Jurkat cells (A, B, C, G) and HCC1806 cells (D, E, F, H) show NMD3 is the primary target candidate of portimine A. Volcano plot in panels A and D show protein competition in cells treated with active photoaffinity probe PA-DA (500 nM) and DMSO or the active competitor portimine A (PA, 4 pM), dotted lines indicate threshold of fold change <4, p-value>0.05. Volcano plots in panels B and E show protein enrichment by active probe PA-DA over inactive probe ePA-DA (500 nM), dotted lines indicate threshold of fold change <2, p-value>0.05. Volcano plot in panels C and F show comparison between competition by active competitor compound PA to inactive control competitor ePA (4 pM, PA-DA+PA/PA-DA+ePA), dotted lines indicate threshold of fold change >4, p-value>0.05. (I, J) Confirmation of PA-DA engagement of NMD3 in Jurkat and HCC1806 cells. UV-dependent labeling of endogenous NMD3 by PA-DA is blocked by PA, but not ePA, and was not substantially labeled by inactive probe ePA-DA or PA-DA with no UV-irradiation. Treatment condition: Jurkat (5M per well). Portimine A concentration: 2 nM-1000 nM; In total treat cell for 12 hours. Conclusion: PA causes cell apoptosis in dose-dependent manner; ePA does not show significant Caspase 3 activation at this concentration range.
6) Portimine A downregulates NMD3 at protein level but upregulates in mRNA level in cells, as well as some essential apoptosis proteins (FIG. 6). (A) NMD3 and Bcl-xl downregulated after PA treatment, and not by proteasome-mediated degradation. Protein degradation of p53, Mcl1, Bid, Bim, Noxa can be rescued by MG132 or Epoxomicin. (B) NMD3 mRNA increased after PA treatment, but not ePA control compound. Jurkat cells were treated with indicated compounds for 24 hr; Cells were then fixed and perform Propidium Iodide staining. Conclusion: S phase entry was inhibited in Jurkat cells with 10 nM treatment of PA/PADA; PA/PADA does not affect G2M/sub-G1 (mostly apoptotic cells).
7) NMD3 is downregulated by PA in Jurkat cells in a time- and dose-dependent manner (FIG. 7). (A) Immunoblot showing reduced NMD3 levels in shNMD3 Jurkat and HeLa cells. (B) PA has less toxic effects in cells where NMD3 is knocked down (shNMD3) compared to shCtrl cells. Jurkat cells were treated with 12.5 nM of PA for 12 or 24 hours. Jurkat and HeLa cells were treated with 5 nM or 150 nM of PA for 24 hours. Data represents mean±SD of biologically replicated experiments (n=3). (C) Polysome profiling reveals the protein translation inhibition by portimine A. Jurkat cells were treated with DMSO or portimine A for 6 hours at 50 nM. Cell lysates were fractionated by sucrose gradient (5-50%) and each fraction were analyzed by western blot. eIF6 levels increase in 60S subunit fraction and decrease in ribosome-free fractions after PA treatment. RPS6, a 40S and polysome subunits marker, is decreased in polysome fractions after PA treatment. Results are representative of three independent experiments. (D) Quantifications of 60S:80S ratio and 80S:polysome ratio from polysome profiling assay as shown in FIG. 7C. (E) Quantification of relative protein distribution in fractions as displayed in FIG. 7C. Relative protein levels in each fraction were normalized to the peak fraction of the indicated protein from the DMSO cells and plotted. Data represents mean±SD of biologically replicated experiments (n=3). Statistical analysis was performed using multiple unpaired Student t-test. * p≤0.05; ** p≤0.01; *** p≤0.005; **** p≤0.0001. Treatment condition: Jurkat (5 M per condition); Incubate cells (1 M/mL) with inhibitors for 6 hr, 12 hr and 24 hr, respectively. Conclusion: NMD3 is downregulated by PA treatment at 10 nM at 12 hrs; KPT-330 downregulates NMD3 at higher (<50 nM) concentration at 24 hrs; XPO1 levels appear unaffected by PA; p53 can be downregulated by Portimine A treatment; Similar to KPT-330, PA does not affect Bcl2 levels at 6/12 hrs.
8) NMD3 down regulation is not occurring through proteosome-mediated degradation (FIG. 8). Treatment condition: Jurkat (10 M per condition); PA concentration: 10 nM; Preincubate cells (1 M/mL) with MG132 or Epoxomicin for 2 hr. Incubate cells with PA with or without MG132 or Epoxomicin for another 12 hr. Conclusion: NMD3 downregulation is not by proteasome-mediated degradation; Similar to XPO1 inhibitors, might be result of transcriptional inhibition.
9) Portimine A displays potent anti-tumor activity in human and syngeneic mouse tumor models (FIG. 9). (A) Systemic Portimine A in MC38 Syngeneic Tumor Xenograft Model; and (B) Systemic Portimine A in HT-1080 Tumor Xenograft Model. Preliminary cell-based target engagement studies, based on compound washout, indicates PA is fast acting with <1 hr of exposure resulting in minimal loss of potency. Cmax-driven pharmacology and facile synthetic access to material enabled the evaluation of PA in both a syngeneic (MC38 colon carcinoma) and a human metastatic mesenchymal (HT-1080 fibrosarcoma) mouse tumor xenograft model. Potent anti-tumor activity observed for PA at 0.3 or 1.0 mg/kg doses, following system administration of tolerated doses (i.p. delivery). Metabolic stability represents an opportunity for an additional IP position.

### Broad Cell Line Profiling Data.

### Portimine a Displays Cytotoxic Activity at pM to Single Digit nM Concentrations Across a Broad Range of Cancer Cells Types

A panel of human and mouse cancer cell types used to assess the breadth of PA activity, including multiple therapy resistant metastatic mesenchymal tumor cell lines and primary glioblastoma patient-derived metastatic stem/tumor initiating cell populations.

| Cell Line | Cell Type | PA IC₅₀, nM* |
|---|---|---|
| Jurkat | Human Leukemia | 0.56 |
| RD | Human Rhabdomyosarcoma | 0.56 |
| HT-1080 | Human Fibrosarcoma | 0.83 |
| A673 | Human Ewings Sarcoma | 2.5 |
| HCC1806 | Acantholytic Squamous Carcinoma | 0.72 |
| HeLa | Cervical Adenocarcinoma | 1.6 |
| MCF-7 | Breast Adenocarcinoma | 0.77 |
| MD-MBA-231 | TNBC Breast Adenocarcinoma | 0.68 |
| SUM159 | Breast Adenocarcinoma | 1.9 |
| HepG2 | Hepatocellular Carcinoma | 4.8 |
| LNCaP | Prostate Carcinoma | 1.7 |
| 786-O | Renal Adenocarcinoma | 1.8 |
| LN229 | Glioblastoma | 1.1 |
| U87EGFRvIII | EGFRvIII Expressing Glioblastoma | 3.4 |
| U118 | Glioblastoma | 5.6 |
| GBM-A | Glioblastoma Cancer Stem Cell | 2.6 |
| GBM-F | Glioblastoma Cancer Stem Cell | 2.6 |
| MC38 | Mouse Colon Carcinoma | 0.55 |
| B16-F10 | Mouse Melanoma | 0.75 |
| 4T1 | Mouse Mammary Carcinoma | 0.63 |

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

1. A process for preparing Portimine A (1), 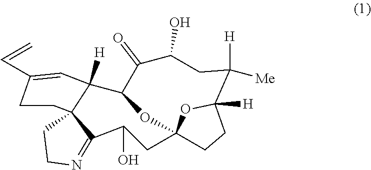
wherein the process comprises the Diels-Alder cycloaddition of Compound (8) to form Compound (10):

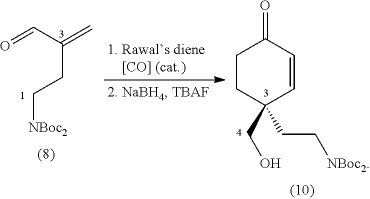

2. The process of claim 1, further comprising the alcohol oxidation of Compound (10), subsequent Grignard addition, and Dess-Martin oxidation to form Compound (11):

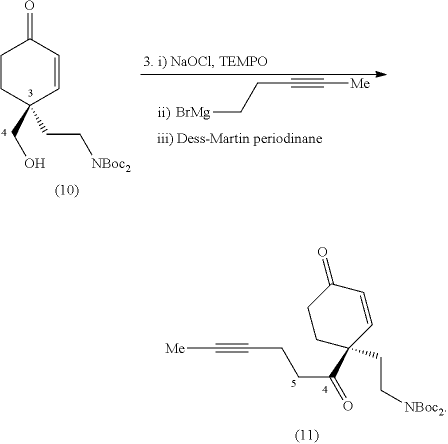

3. The process of claim 2, further comprising the cyclization of Compound (11) to form Compound (12):

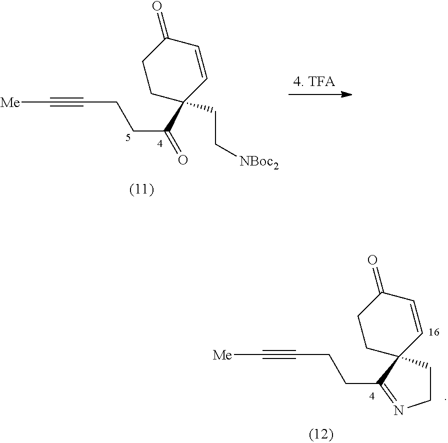

4. A process of preparing Compound (13) from (S)-solketal, comprising the following steps:

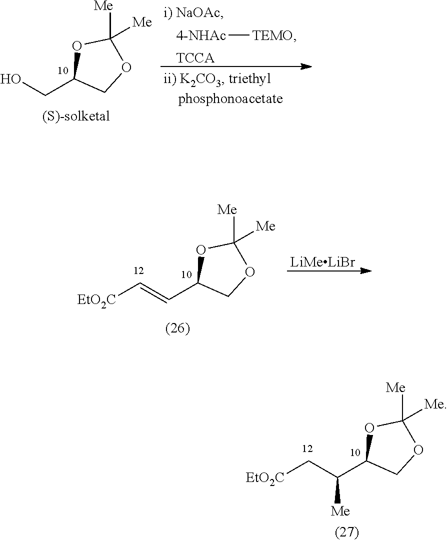

5. The process of claim 4, further comprising the following steps:

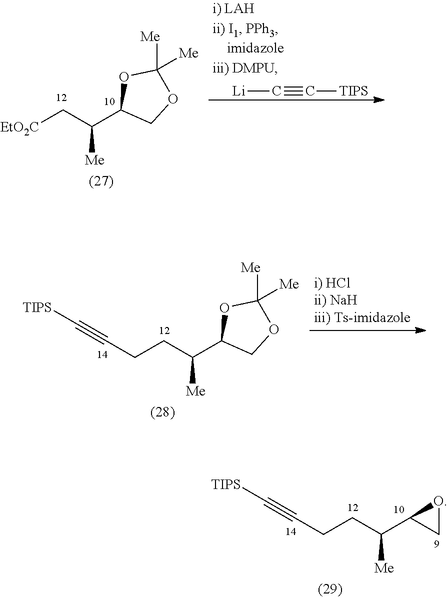

6. The process of claim 5, further comprising the following steps:

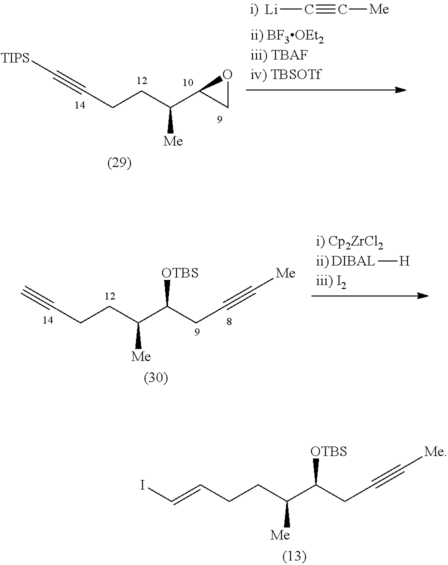

7. The process of claim 3, further comprising the transmetallation of Compound (13) and conjugate addition to Compound (12) to form Compound (7):

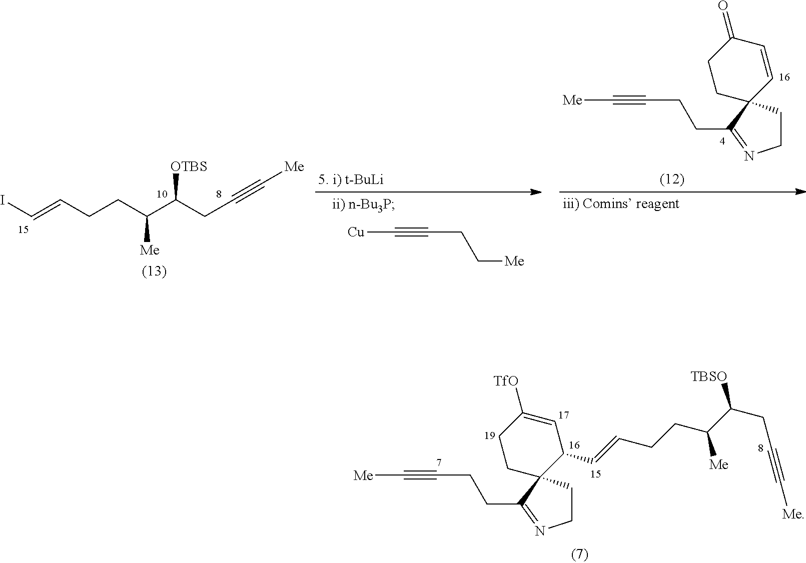

8. The process of claim 7, further comprising the conversion of compound (7) to the N-protected imine Compound (31):

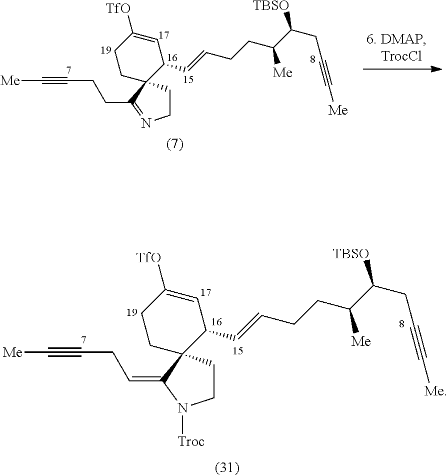

9. The process of claim 8, further comprising Mo-catalyzed ring-closing alkyne metathesis to form Compound (15):

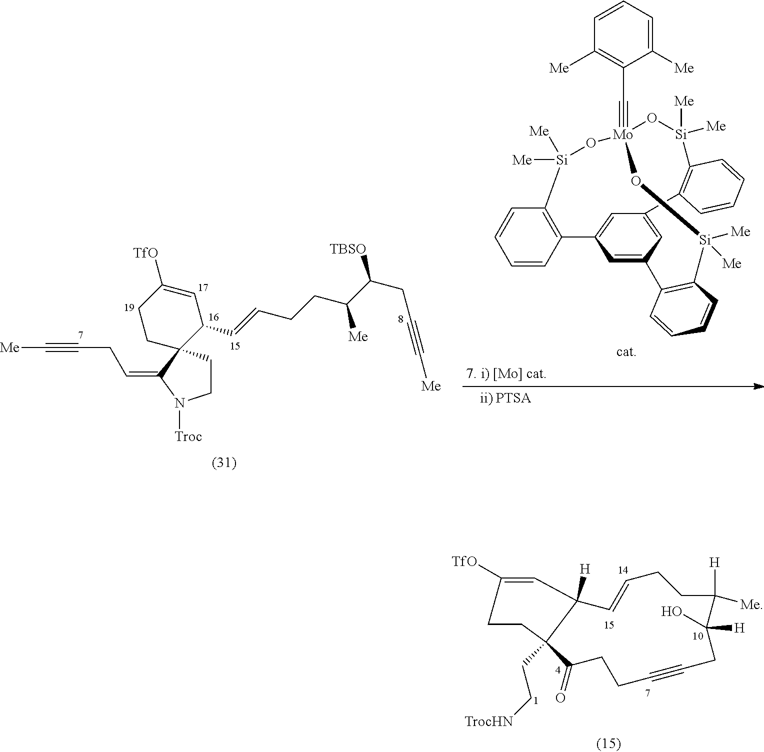

10. The process of claim 9, further comprising ring formation and oxidation of Compound (15) to form Compound (16):

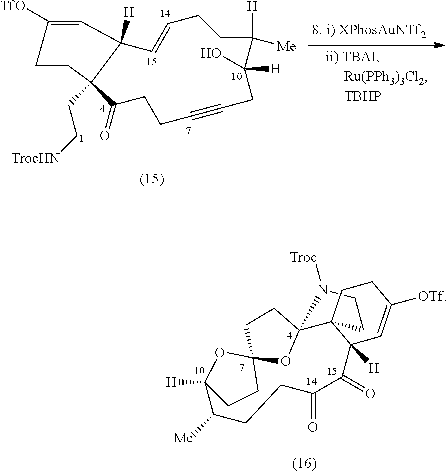

11. The process of claim 10, further comprising the reduction of Compound (16) to Compound (32):

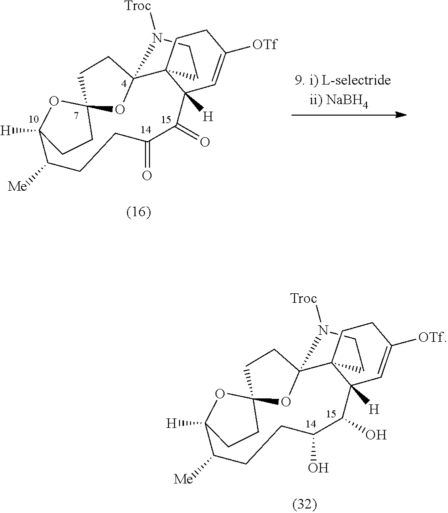

12. The process of claim 11, further comprising ring-chain tautomerism of Compound (32) to form Compound (19):

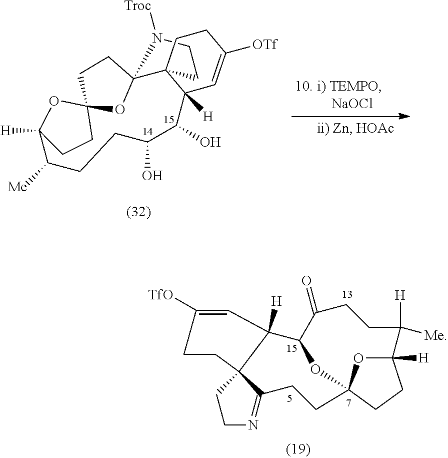

13. The process of claim 12, further comprising the silylation and oxidation of Compound (19) to form Compound (20):

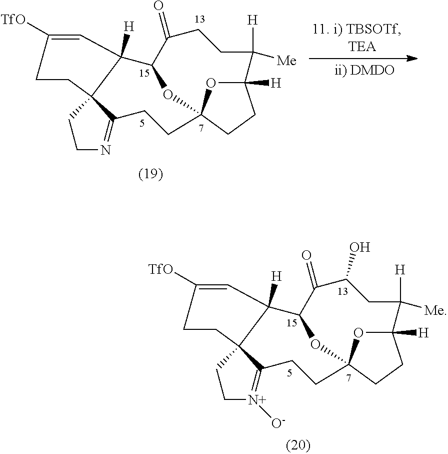

14. The process of claim 13, further comprising the conversion of Compound (20) to Compound (21):

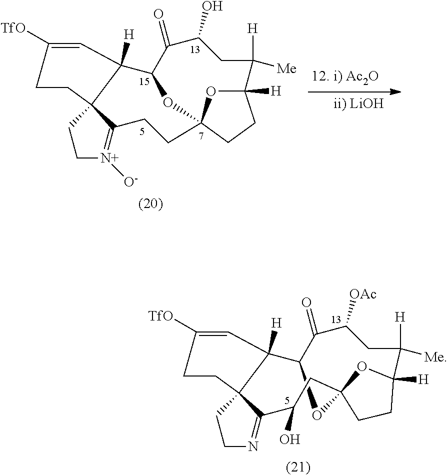

15. The process of claim 14, further comprising Pd-catalyzed vinylation of Compound (21) to form Compound (22):

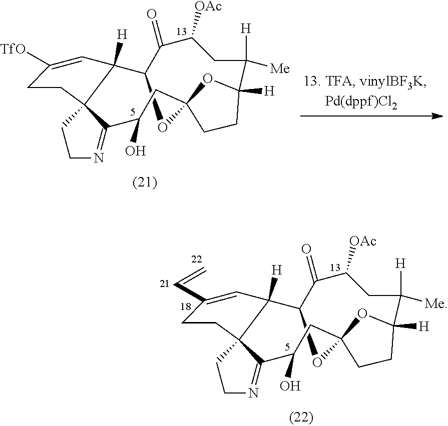

16. The process of claim 15, further comprising the Dess-Martin oxidation of Compound (22) and subsequent hydrolysis to form Compound (2):

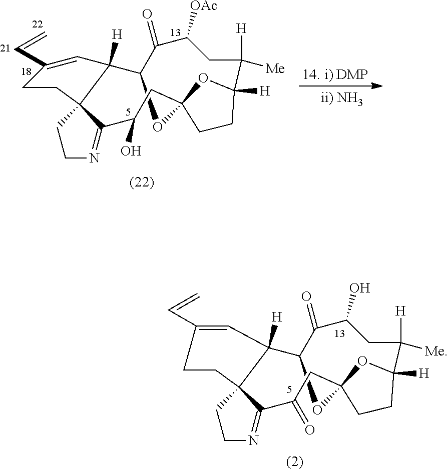

17. The process of claim 16, further comprising the reduction of Compound (2) to form Portimine A (1):

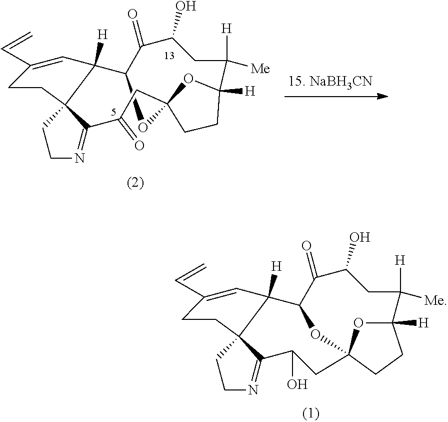

18. A process for preparing Portimine A (1), 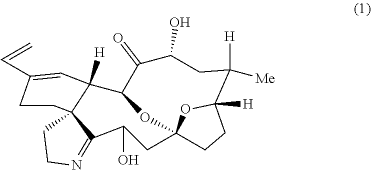
wherein the process comprises the reduction of Compound (2) to form Portimine A (1):

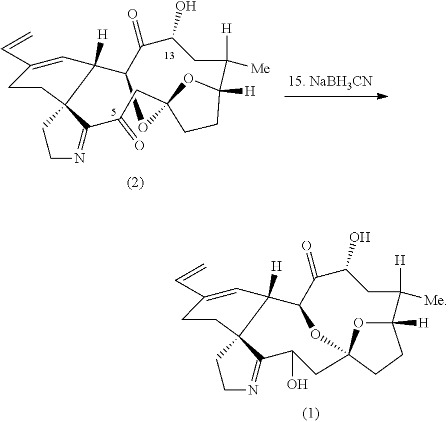

19. A compound having the structure of Formula (2): 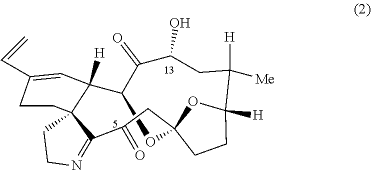
or a scalemic or racemic mixture of.

20. A process of preparing the compound of claim 19, comprising the Dess-Martin oxidation of Compound (22) and subsequent hydrolysis to form Compound (2) [chemical structure]
